(19) United States
(12) Patent Application Publication
(10) Pub. No.: US 2008/0220471 A1
(43) Pub. Date: Sep. 11, 2008

(54) Vectors and Methods Using Same

(75) Inventors: David P. Davis, San Bruno, CA (US); Daniel C. Gray, San Francisco, CA (US); Klaus P. Hoeflich, San Mateo, CA (US); Somasekar Seshagiri, San Carlos, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/460,606

(22) Filed: Jul. 27, 2006

Related U.S. Application Data
(60) Provisional application No. 60/702,939, filed on Jul. 27, 2005.

(51) Int. Cl. C12P 21/06; C07H 21/00; C12N 15/00; C12N 5/00

(52) U.S. Cl. 435/69.1; 536/24.1; 435/320.1; 435/325

(57) ABSTRACT

The present invention is directed to vectors and to methods of using same.

### CROSS-REFERENCE TO RELATED APPLICATION

This application claims priority under 35 USC § 119 to U.S. Provisional Application 60/702,939, filed Jul. 27, 2005, the entire contents of which is hereby incorporated by reference.

### FIELD OF THE INVENTION

The present invention is directed to vectors and methods using same. Some embodiments comprise compositions of matter useful for modulating gene expression in mammals and to methods of using those compositions of matter for the same.

### BACKGROUND OF THE INVENTION

The ability to reversibly regulate gene expression has great utility for the analysis of gene expression and function, particularly for those genes whose products are toxic to the cell. Accordingly, there is a great need for methods for efficiently and reliably modulating gene expression.

Mammalian genetic studies have been hampered to date by the lack of success in efficiently generating stable loss-of-function phenotypes. The ability to determine the impact caused in a living organism by lack of expression of a particular gene product promises to greatly facilitate understanding of mammalian gene regulation and gene function. This ability to dissect mammalian genetic pathways will ultimately enable the identification of targets for therapeutic interventions aimed at compensating for genetic deficiencies.

RNA interference holds promise as a method for the modulation of gene expression. However, RNA interference methods have been hampered by the lack of reliable methods for the efficient, regulatable delivery of RNA molecules.

The ability to achieve reliable and efficient inducible delivery of RNA molecules (such as siRNA) to cellular systems would render RNA interference a powerful functional genomics tool by providing the ability to inhibit expression of target genes to see what effect their absence has on the cell or organism. Furthermore, the ability to selectively inhibit target gene expression has important therapeutic implications and could be useful to prevent the production of proteins that are harmful to the body.

Thus, there exists a need for creating methods for efficiently and reliably delivering RNA molecules to cellular systems. The present invention satisfies this need and provides related advantages as well.

Malignant tumors (cancers) are the second leading cause of death in the United States, after heart disease (Boring et al., CA Cancer J. Clin. 43:7 (1993)). Cancer is characterized by the increase in the number of abnormal, or neoplastic, cells derived from a normal tissue which proliferate to form a tumor mass, the invasion of adjacent tissues by these neoplastic tumor cells, and the generation of malignant cells which eventually spread via the blood or lymphatic system to regional lymph nodes and to distant sites via a process called metastasis. In a cancerous state, a cell proliferates under conditions in which normal cells would not grow. Cancer manifests itself in a wide variety of forms, characterized by different degrees of invasiveness and aggressiveness.

In attempts to discover effective cellular targets for cancer therapy, researchers have sought to identify polypeptides that are specifically overexpressed in a particular type of cancer cell as compared to on one or more normal non-cancerous cell(s). The identification of such tumor-associated cell polypeptides has given rise to the ability to specifically target cancer cells for destruction via antibody-based therapies. In this regard, it is noted that antibody-based therapy has proved very effective in the treatment of certain cancers. For example, HERCEPTIN® and RITUXAN® (both from Genentech Inc., South San Francisco, Calif.) are antibodies that have been used successfully to treat breast cancer and non-Hodgkin's lymphoma, respectively. More specifically, HERCEPTIN® is a recombinant DNA-derived humanized monoclonal antibody that selectively binds to the extracellular domain of the human epidermal growth factor receptor 2 (HER2) proto-oncogene. HER2 protein overexpression is observed in 25-30% of primary breast cancers. RITUXAN® is a genetically engineered chimeric murine/human monoclonal antibody directed against the CD20 antigen found on the surface of normal and malignant B lymphocytes.

The kinases that control signal transduction pathways, cell cycle and programmed cell death are critical to cell regulation. Overexpression or activating mutations of these critical kinases may disrupt cellular regulation and lead to tumor formation. Twenty percent of all known oncogenes are protein kinases. Identifying the appropriate signal transduction pathway and developing drugs to specifically inhibit these oncogenic kinases has been a major goal of cancer research for some time. High throughput screening has led to identification of small molecules with different modes of inhibition such as; competition with the catalytic adenosine triphosphate binding site, inhibition of substrate binding, or modification the substrate itself. Certain compounds are highly specific for a single kinase, while others can inhibit several kinases with similar binding structures (Busse et al., Semin. Oncol. 28:47-55 (2001)). For example, the tyrosine kinase Bcr-Abl has been identified as a causative factor in chronic myeloid leukemia (CML). The small molecule imatinib mesylate (Gleevec-Novartis Pharmaceuticals Corp, East Hanover, N.J.) was recently approved for the treatment of CML, demonstrating that treatment of the kinase component of a signal transduction pathway is effective in the treatment of cancer (Griffin J. Semin. Oncol. 28:3-8 (2001)).

Raf serine/threonine kinases are downstream effector molecules of Ras (Mercer et al., Biochem. Biophys. Acta 1653:25-40 (2003)). The Raf family is comprised of A-Raf, B-Raf and C-Raf, which have a high degree of homology within three conserved regions. Two of these regions are a Ras-binding domain and a cysteine-rich domain that interacts with GTP-complexed Ras (Vojtek et al., Cell 74:205-14 (1993)). When Raf and Ras bind, Raf is recruited to plasma membrane and inhibition of the Raf catalytic domain is relieved and leads to phosphorylation of specific sites within the activation domain of B-Raf, and hence activation of the molecule. In contrast, both A-Raf and C-Raf require additional phosphorylation outside of the catalytic domain. This distinct and simple activation mechanism of B-Raf makes it an important regulator of downstream targets in the pathway such as MEK. In B-Raf knockout mice, MEK activation was disrupted, but this result is not seen in cells that lack either A-Raf or C-Raf. (Pritchard et al., Mol. Cell. Bio. 24:5937-5952 (2004) Mercer et al., Oncogene 21:347-355 (2002)). B-Raf's predominant role in MEK activation is believed to be the reason that B-Raf mutations are associated with cancer (Wellbrock et al., Nature Rev. 5 875-885 (2004)).

B-Raf's incidence in cancer is reported to be highest in malignant melanoma (27%-70%), papillary thyroid cancer (36%-53%), colorectal cancer (5%-22%) and serous ovarian cancer (about 30%) (Garnett et al., Cancer Cell 6:313-319 (2004)). Approximately 90% of B-Raf mutations are a transversion in exon 15 which results in a valine to glutamic acid change at residue 600 (V600E). The importance of B-Raf in the RAF/MEK/ERK signaling pathway and its predominance in certain tumors make B-Raf an attractive therapeutic target for kinase inhibitors. The kinase inhibitor BAY 43-9006 was developed as a Raf kinase inhibitor although it inhibits other kinases such as VEGFR2, VEGFR3, PDGFRβ, FLT3, C-KIT and P38 MAPK (Wilhelm et al., Cancer Res. 64; 7099-7109 (2004)). BAY 43-9006 is currently in clinical testing, but a correlation between BAY 43-9006 efficacy and the prevalence of B-Raf mutation has yet to be established. The example of BAY 43-9006 teaches that the most important consideration for therapeutic design is the choice of the target. The development of new tools to define an oncology target is useful and one of the better methods to define an oncology target is to modulate its expression.

Despite advances in mammalian cancer therapy, there is a great need for therapeutic agents capable of effectively inhibiting neoplastic cell growth through reduction of gene expression. Accordingly, it is an objective of the present invention to identify therapeutic targets and methods of modulating those targets.

All references cited herein, including patent applications and publications, are incorporated by reference in their entirety.

### SUMMARY OF THE INVENTION

In one aspect, this invention generally relates to vectors that permit the regulated, inducible expression of a RNA coding sequence of a polypeptide. The vectors generally include a polynucleotide encoding the Tet repressor (TetR) protein and a promoter comprising one or more Tet operon (TetO) sequences. As demonstrated herein, use of inducible vectors comprising a RNA coding sequence of the invention permitted tightly regulated, reversible and stable target gene knockdown in mammalian cells, and embryonic cells. Surprisingly, titration of level of TetR repression resulted in a titration of the level of RNA interference observed. Use of the inducible vectors also permitted regulatable expression of a protein of interest.

In another aspect, the invention generally relates to a codon-optimized TetR coding region, and vectors comprising this codon-optimized TetR coding region. As demonstrated in the Examples, use of the codon optimized TetR increased TetR protein expression and permitted tight control of inducible gene expression, for example, by minimizing undesired expression of a RNA coding region (and thus undesired RNA interference) in the absence of induction agent. In addition, use of the codon optimized TetR permitted induction of gene expression at lower levels of induction agent.

In another aspect, the invention generally relates to a modified H1 promoter comprising two Tet operon sites, and vector comprising this modified H1 promoter. Use of this promoter permitted enhanced control over TetR-mediated repression particularly in conditions where TetR expression was limiting. This promoter showed particular utility in embryonic stem cells and EB cells, in which TetR-mediated repression was significantly more sensitive and stronger when the H1 promoter with two Tet operons was used.

In one aspect, the invention provides polynucleotides comprising a polynucleotide encoding a codon optimized Tet repressor (TetR) protein and a promoter comprising a Tet operon.

In another aspect, the invention provides polynucleotides comprising a polynucleotide encoding a codon optimized Tet repressor (TetR) protein and a promoter comprising a Tet operon, wherein the promoter is operably linked to a RNA coding sequence.

In another aspect, the invention provides polynucleotides comprising: (a) a first transcription unit comprising a RNA polymerase III promoter, wherein the RNA polymerase III promoter comprises a polynucleotide sequence shown in. SEQ ID NO:19, and wherein the RNA polymerase III promoter is operably linked to a RNA coding sequence; and (b) a second transcription unit comprising a second promoter, wherein the second promoter is operably linked to (1) a polynucleotide encoding a TetR protein, and (2) a selectable marker.

In another aspect, the invention provides polynucleotides comprising: (a) a first transcription unit comprising a RNA polymerase II promoter, wherein the RNA polymerase II promoter comprises a polynucleotide sequence shown in SEQ ID NO:19, and wherein the RNA polymerase II promoter is operably linked to a RNA coding sequence; and (b) a second transcription unit comprising a second promoter, wherein the second promoter is operably linked to (1) a polynucleotide encoding a TetR protein, and (2) a selectable marker.

In another aspect, the invention provides polynucleotides comprising: (a) a first transcription unit comprising a RNA polymerase II promoter, wherein the RNA polymerase II promoter comprises a polynucleotide sequence shown in SEQ ID NO:19, and wherein the RNA polymerase II promoter is operably linked to a polynucleotide encoding a selected sequence; and (b) a second transcription unit comprising a second promoter, wherein the second promoter is operably linked to (1) a polynucleotide encoding a TetR protein, and (2) a selectable marker.

In another aspect, the invention provides polynucleotides comprising: (a) a first transcription unit comprising a first promoter comprising a tet operon, wherein the first promoter is operably linked to a RNA coding sequence; and (b) a second transcription unit comprising a second promoter, wherein the second promoter is operably linked to (1) a polynucleotide encoding a TetR protein, and (2) a selectable marker.

In another aspect, the invention provides polynucleotides comprising: (a) a first transcription unit comprising a first promoter comprising a tet operon, wherein the first promoter is operably linked to a polynucleotide encoding a selectable sequence; and (b) a second transcription unit comprising a second promoter, wherein the second promoter is operably linked to (1) a polynucleotide encoding a TetR protein and (2) a selectable marker.

In another aspect, the invention provides polynucleotides comprising: (a) a first transcription unit comprising a first RNA polymerase II promoter comprising a tet operon, wherein the first promoter is operably linked to a polynucleotide encoding a selectable sequence; and (b) a second transcription unit comprising a second promoter, wherein the second promoter is operably linked to (1) a polynucleotide encoding a TetR protein and (2) a selectable marker.

In another aspect, the invention provides polynucleotides comprising: (a) a first transcription unit comprising a first RNA polymerase II promoter comprising a tet operon, wherein the first promoter is operably linked to a polynucleotide encoding a miRNA; and (b) a second transcription unit comprising a second promoter, wherein the second promoter is operably linked to (1) a polynucleotide encoding a TetR protein and (2) a selectable marker.

In another aspect, the invention provides polynucleotides comprising: (a) a first transcription unit comprising a first RNA polymerase III promoter comprising a tet operon, wherein the first promoter is operably linked to a polynucleotide encoding a selectable sequence; and (b) a second transcription unit comprising a second promoter, wherein the second promoter is operably linked to (1) a polynucleotide encoding a TetR protein and (2) a selectable marker.

In another aspect, the invention provides polynucleotides comprising: (a) a first transcription unit comprising a H1 promoter comprising a tet operon, wherein the H1 promoter is operably linked to a polynucleotide encoding a selectable sequence; and (b) a second transcription unit comprising a second promoter, wherein the second promoter is operably linked to (1) a polynucleotide encoding a TetR protein and (2) a selectable marker.

In another aspect, the invention provides polynucleotides comprising, in order from 5′ to 3′: a retroviral 5′ LTR; a first promoter, wherein the first promoter is operably linked to a RNA coding region; a second promoter, wherein the second promoter is operably linked to (a) a polynucleotide encoding a TetR and (b) a polynucleotide encoding a selectable marker; and a retroviral 3′ LTR.

In another aspect, the invention provides polynucleotides comprising, in order from 5′ to 3′: a retroviral 5′ LTR; a first promoter, wherein the second promoter is operably linked to (a) a polynucleotide encoding a TetR and (b) a polynucleotide encoding a selectable marker; a second promoter, wherein the first promoter is operably linked to a RNA coding region; and a retroviral 3′ LTR.

In another aspect, the invention provides polynucleotides comprising, in order from 5′ to 3′: a retroviral 5′ LTR; a first promoter, wherein the first promoter is operably linked to polynucleotide encoding a selected sequence; a second promoter, wherein the second promoter is operably linked to (a) a polynucleotide encoding a TetR and (b) a polynucleotide encoding a selectable marker; and a retroviral 3′ LTR.

In another aspect, the invention provides polynucleotides comprising, in order from 5′ to 3′: a retroviral 5′ LTR; a first promoter, wherein the second promoter is operably linked to (a) a polynucleotide encoding a TetR and (b) a polynucleotide encoding a selectable marker; a second promoter, wherein the first promoter is operably linked to a selected sequence; and a retroviral 3′ LTR.

In some embodiments, the second transcription unit comprises, in order from 5′ to 3′: a promoter, a polynucleotide encoding a TetR, an IRES, and a polynucleotide encoding a selectable marker. In some embodiments, the second transcription unit comprises, in order from 5′ to 3′: a promoter, a polynucleotide encoding a selectable marker, an IRES, and a polynucleotide encoding a TetR.

In some embodiments, the codon optimized TetR is optimized for mammalian expression. In some embodiments, the codon optimized TetR is optimized for human expression. In some embodiments, the polynucleotide encoding codon optimized TetR is: (a) a polynucleotide shown in FIG. 17 (SEQ ID NO:1); (b) a polynucleotide comprising a polynucleotide that hybridizes under stringent conditions to the compliment of a polynucleotide shown in FIG. 17 (SEQ ID NO:1); or (c) a polynucleotide comprising a polynucleotide that is 90% identical to a polynucleotide shown in FIG. 17 (SEQ ID NO:1). Preferably, the polypeptide encoded by the polynucleotide of (a), (b) or (c) is capable of binding Tet operon.

The promoter comprising one or more tet operon(s) (such as 2, 3, or more tet operons) can be a RNA polymerase III (pol III) promoter. In some embodiments, the promoter is selected from the group consisting of the H1 promoter or the U6 promoter. In some embodiments, the promoter comprising a tet operon comprises (a) a polynucleotide shown in SEQ ID NO:19; (b) a polynucleotide comprising a polynucleotide that hybridizes under stringent conditions to the compliment of a polynucleotide shown in SEQ ID NO:19; or (c) a polynucleotide comprising a polynucleotide that is 90% identical to a polynucleotide shown in SEQ ID NO:19. Preferably, the polynucleotide of (a), (b) or (c) is capable of being bound by TetR protein. In other embodiments, the promoter is a RNA polymerase II (pol II) promoter, such as the CMV promoter.

In one aspect, the invention provides retroviral constructs for the expression of an RNA molecule or molecules within a cell. The constructs preferably comprise a nucleic acid having the R and U5 sequences from a 5′ lentiviral long terminal repeat (LTR), a self-inactivating lentiviral 3′ LTR, and a RNA Polymerase III (po III) promoter. The retroviral constructs preferably comprise an RNA coding region operably linked to the RNA Polymerase III promoter. The RNA coding region preferably comprises a DNA sequence that can serve as a template for the expression of a desired RNA molecule. According to one embodiment of the invention, the 5′ LTR sequences in the retroviral construct are derived from HIV. The retroviral construct may also comprise a woodchuck hepatitis virus enhancer element sequence and/or a tRNA amber suppressor sequence. In another embodiment of the invention, the self-inactivating 3′ LTR is a U3 element with a deletion of its enhancer sequence. In yet another embodiment, the self-inactivating 3′ LTR is a modified HIV 3′ LTR. The recombinant retroviral construct can be pseudotyped, for example with the vesicular stomatitis virus envelope glycoprotein.

The RNA coding sequence can encode a self-complimentary RNA molecule having a sense region, an antisense region and a loop region. In some embodiments, the sense region and antisense region are each between about 15 and 30 nucleotides in length and the loop region is about 2 to about 15 nucleotides in length. In some embodiments, the RNA coding sequence encodes a RNA molecule selected from a shRNA, a miRNA, or a siRNA.

The RNA coding region can be immediately followed by a pol III terminator sequence which directs the accurate and efficient termination of RNA synthesis by pol III. The pol III terminator sequences generally comprise 4 or more consecutive T residues. In a preferred embodiment, a cluster of 5 consecutive Ts is used as the terminator by which pol III transcription is stopped at second or third T of the DNA template. As a result, only 2 to 3 U residues are added to the 3′ end of the RNA that is synthesized from the RNA coding region.

A variety of pol III promoters can be used with the invention, including for example, the promoter fragments derived from H1 RNA genes or U6 sn RNA genes of human or mouse origin or from any other species. In some embodiments, the promoter is selected from the group consisting of the H1 promoter or the U6 promoter. In some embodiments, the promoter comprising a tet operon comprises (a) a polynucleotide shown in SEQ ID NO:19; (b) a polynucleotide comprising a polynucleotide that hybridizes under stringent conditions to the compliment of a polynucleotide shown in SEQ ID NO:19; or (c) a polynucleotide comprising a polynucleotide that is 90% identical to a polynucleotide shown in SEQ ID NO:19. Preferably, the polynucleotide of (a), (b) or (c) is capable of being bound by TetR protein.

In one embodiment, the RNA coding region encodes a self-complementary RNA molecule having a sense region, an antisense region and a loop region. Such an RNA molecule when expressed desirably forms a “hairpin” structure. The loop region is generally between about 2 and about 10 nucleotides in length. In a preferred embodiment, the loop region is from about 6 and about 9 nucleotides in length. In one such embodiment of the invention, the sense region and the antisense region are between about 15 and about 30 nucleotides in length. In some embodiments, the RNA coding region encodes a siRNA, a shRNA, a miRNA, or an antisense RNA.

In one embodiment, the RNA coding region is operably linked downstream to an RNA Polymerase III promoter such that the RNA coding sequence can be precisely expressed without any extra non-coding nucleotides present at 5′ end. In this way an RNA sequence can be expressed that is identical to a target sequence at the 5′ end. The synthesis of the RNA coding region is ended at the terminator site. In one preferred embodiment the terminator consists of five consecutive T residues.

In another aspect of the invention, the polynucleotides of the invention can comprise multiple RNA coding regions. In one embodiment, the retroviral construct comprises a first RNA pol III promoter, a first coding region encoding a first RNA molecule operably linked to the first RNA pol III promoter, a second RNA pol III promoter and a second RNA coding region operably linked to the second RNA pol III promoter. Preferably, the second RNA coding region encodes an RNA molecule that is substantially complementary to the RNA molecule encoded by the first RNA coding region, such that the two RNA molecules can form a double-stranded structure when expressed. The methods of invention also include multiple RNA coding regions that encode hairpin-like self-complementary RNA molecules or other non-hairpin molecules.

In yet another embodiment of the invention, the polynucleotides of the invention comprise a first RNA pol III promoter operably linked to a first RNA coding region, and a second RNA pol III promoter operably linked to the same first RNA coding region in the opposite direction, such that expression of the RNA coding region from the first RNA pol III promoter results in a synthesis of a first RNA molecule as the sense strand and expression of the RNA coding region from the second RNA pol III promoter results in synthesis of a second RNA molecule as an antisense strand that is substantially complementary to the first RNA molecule. In one such embodiment, both RNA Polymerase III promoters are separated from the RNA coding region by termination sequences, preferably termination sequences having five consecutive T residues.

In another aspect of the invention, expression of the RNA coding region results in the down regulation of a target gene (e.g., at least a 10%, 20%, 30%, 40%, 50%, 60%, 70%, 80%, 90% or more, such as 95%, 96%, 97%, 98% or 99% reduction in target gene expression, when compared with target gene expression in the absence of expression of the RNA coding region).

According to a further aspect of the invention, the polynucleotides of the invention also comprise a nucleotide sequence encoding a gene of interest. The gene of interest is preferably operably linked to a Polymerase II promoter. Such a construct also can contain, for example, an enhancer sequence operably linked with the Polymerase II promoter.

A variety of Polymerase II promoters can be used with the invention, including for example, the CMV promoter. The RNA Polymerase II promoter that is chosen can be a ubiquitous promoter, capable of driving expression in most tissues, for example, the human Ubiquitin-C promoter, CMV, human beta-actin promoter or PGK promoter. In other embodiments the RNA Polymerase II promoter is a tissue-specific promoter.

In one embodiment, the gene of interest (interchangeably termed “selected sequence”) is a marker or reporter gene, that can be used to verify that the vector was successfully transfected or transduced and its sequences expressed. In one such embodiment, the gene of interest is a fluorescent reporter gene, for example, the Green Fluorescent Protein. In yet another embodiment, the gene of interest is a drug resistant gene which can be used to select the cells that are successfully transduced. For example, the drug resistant gene can be the zeocin resistant gene (zeo). The gene of interest also can be a hybrid of a drug resistant gene and a fluorescent reporter gene, such as a zeo/gfp fusion. In another embodiment, the gene of interest encodes a protein factor that can regulate the transcription activity of inducible pol III promoters. In one of such embodiment, the gene of interest is tetR (repressor for tet operon) which regulates tetracycline responsive pol III promoters.

In another aspect, the invention provides vectors comprising any of the polynucleotides disclosed herein. In some embodiments, the vector is an expression vector.

In another aspect, the invention provides a cell comprising any of the polynucleotides disclosed herein. In one embodiment of the invention, the cell is an embryonic cell, such as an ES cell. An embryonic cell may be, for example, a single cell embryo or embryonic cells from within an early-stage embryo. In another embodiment of the invention, the target cell is an embryonic stem cell. When the target cell is an embryonic cell, in one embodiment the embryonic cell is infected by, injecting the recombinant retrovirus between the zona pellucida and the cell membrane of the embryonic cell. In another embodiment, the embryonic cell is infected by removing the zona pellucida and incubating the cell in solution containing the recombinant retrovirus. In such an embodiment, the zona pellucida can be removed, for example, by enzymatic digestion. When the cell is an embryonic cell or an ES cell, the cell may be transplanted in a pseudopregnant female to generate a transgenic animal. When the cell is a cancer cell, the cell may be transplanted into a suitable recipient, e.g., nude mouse, for creation of a xenograft model.

It is another aspect of the invention to provide methods for expressing an RNA molecule or molecules within a cell. Accordingly the invention provides methods for expressing a RNA molecule within a cell, said methods comprising: introducing any of the polynucleotides described herein comprising a RNA coding region into the cell under conditions permitting expression of the RNA coding region. In some embodiments, the invention provides methods for the regulatable expression of a RNA molecule within a cell, the methods comprising (a) introducing a polynucleotide (such as any suitable polynucleotide described herein) into a cell, wherein the polynucleotide comprises (1) a polynucleotide encoding a TetR protein, and (2) a promoter operably linked to a RNA coding region; and (b) treating the cell with an induction agent (such as doxycycline (or other suitable agent that binds TetR protein and causes it to release from the tet operon), whereby the promoter directs expression of the RNA molecule within the cell. In some embodiments, the invention provides methods for the regulatable expression of a RNA molecule within a cell, the methods comprising: treating a cell with doxycycline (or other agent), wherein the cell comprises a polynucleotide comprising a polynucleotide encoding a TetR protein, and a promoter operably linked to a RNA coding region (such as any suitable polynucleotide described herein), whereby the promoter directs expression of the RNA molecule within the cell.

It is another aspect of the invention to provide methods for the regulatable expression of a selected sequence within a cell. Accordingly the invention provides methods for the regulatable expression of a RNA molecule within a cell, the methods comprising (a) introducing a polynucleotide (such as any suitable polynucleotide described herein) into the cell, wherein the polynucleotide comprises (1) a polynucleotide encoding a TetR protein, and (2) a promoter operably linked to a polynucleotide encoding a selected sequence; and (b) treating the cell with an induction agent (such as doxycycline or other suitable agent that binds TetR protein and causes it to release from the tet operon), whereby the promoter directs expression of the selected sequence within the cell. In some embodiments, the invention provides methods for the regulatable expression of a polypeptide (selected sequence) within a cell, the methods comprising: treating the cell with doxycycline (or other agent), wherein the cell comprises a polynucleotide (such as any suitable polynucleotide described herein) comprising (1) a polynucleotide encoding a TetR protein, and (2) a promoter operably linked to a polynucleotide encoding a selected sequence, whereby the promoter directs expression of the selected sequence within the cell.

In another aspect, the invention provides methods for generating retroviral vector, the methods comprising: introducing a retroviral vector (such as any suitable retroviral vector disclosed herein) into a packaging cell line, and recovering retroviral particles from the packaging cell line.

In another aspect, the invention provides methods for introducing a retrovirus into a cell, the methods comprising introducing retroviral particles into the cell, wherein the retroviral particles comprise any of the polynucleotides described herein.

In another aspect, the invention provides a method of producing a non-human mammal in which the expression of a target gene is inhibited by (a) introducing any of the polynucleotides described herein into a pre-implantation mammalian embryo or ES cell, (b) transferring the pre-implantation embryo or ES cell into a non-human recipient mammal; and (c) allowing the embryo to develop into at least one viable mammal.

In another aspect, the invention provides polynucleotides comprising: (a) a polynucleotide shown in FIG. 17 (SEQ ID NO:1); (b) a polynucleotide comprising a polynucleotide that hybridizes under stringent conditions to the compliment of a polynucleotide shown in FIG. 17 (SEQ ID NO:1); (c) a polynucleotide comprising a polynucleotide that is 90% identical to a polynucleotide shown in FIG. 17 (SEQ ID NO:1); or (d) a complement of a polynucleotides shown in (a), (b) or (c). Preferably, the polypeptide encoded by the polynucleotide of (a), (b) or (c) is capable of binding Tet operon. Binding to tet operon may be determined using standard methods known in the art. The polynucleotides are suitable for use in any of the methods of vectors described herein.

In another aspect, the invention provides polynucleotides comprising: (a) a polynucleotide shown in SEQ ID NO:19; (b) a polynucleotide comprising a polynucleotide that hybridizes under stringent conditions to the compliment of a polynucleotide shown in SEQ ID NO:19; (c) a polynucleotide comprising a polynucleotide that is 90% identical to a polynucleotide shown in SEQ ID NO:19; or (d) a complement of the polynucleotide shown in (a), (b) or (c). Preferably, the polynucleotide of (a), (b) or (c) is capable of being bound by TetR protein. Binding by TetR protein can be determined using standard methods known in the art. The polynucleotides are suitable for use in any of the methods of vectors described herein.

An aspect of the invention provides an isolated nucleic acid molecule comprising a nucleotide sequence encoding a TUMOR-ASSOCIATED KINASE-121 (interchangeably termed B-RAF) polypeptides (“TASK-121” polypeptide).

In certain aspects, the isolated nucleic acid molecule comprises a nucleotide sequence having at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% nucleic acid sequence identity, to (a) a DNA molecule encoding a full-length TASK polypeptide having an amino acid sequence as disclosed herein, or any other specifically defined fragment of a full-length TASK polypeptide amino acid sequence as disclosed herein, or (b) the complement of the DNA molecule of (a).

In other aspects, the isolated nucleic acid molecule comprises a nucleotide sequence having at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% nucleic acid sequence identity, to (a) a DNA molecule comprising the coding sequence of a full-length TASK polypeptide cDNA as disclosed herein, or the coding sequence of any other specifically defined fragment of the full-length TASK polypeptide amino acid sequence as disclosed herein, or (b) the complement of the DNA molecule of (a).

In further aspects, the invention concerns an isolated nucleic acid molecule comprising a nucleotide sequence having at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% nucleic acid sequence identity, to a DNA molecule that encodes the same mature polypeptide encoded by the full-length coding sequence of any of the human protein cDNAs as disclosed herein. In this regard, the term “full-length coding sequence” refers to the TASK polypeptide-encoding nucleotide sequence of the cDNA (which is often shown between start and stop codons, inclusive thereof, in the accompanying figures).

In other aspects, the present invention is directed to isolated nucleic acid molecules which hybridize to (a) a nucleotide sequence encoding a TASK polypeptide having a full-length amino acid sequence as disclosed herein, or any other specifically defined fragment of a full-length TASK polypeptide amino acid sequence as disclosed herein, or (b) the complement of the nucleotide sequence of (a). In this regard, an embodiment of the present invention is directed to fragments of a full-length TASK polypeptide coding sequence, or the complement thereof, as disclosed herein, that may find use as, for example, hybridization probes useful as, for example, diagnostic probes, antisense oligonucleotide probes, or for encoding fragments of a full-length TASK polypeptide. Such nucleic acid fragments are usually at least about 5 nucleotides in length, alternatively at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length. It is noted that novel fragments of a TASK polypeptide-encoding nucleotide sequence may be determined in a routine manner by aligning the TASK polypeptide-encoding nucleotide sequence with other known nucleotide sequences using any of a number of well known sequence alignment programs and determining which TASK polypeptide-encoding nucleotide sequence fragment(s) are novel. All of such novel fragments of TASK polypeptide-encoding nucleotide sequences are contemplated herein. Also contemplated are the TASK polypeptide fragments encoded by these nucleotide molecule fragments, preferably those TASK polypeptide fragments that comprise a binding site for a TASK binding oligopeptide or other small molecule that binds to a TASK polypeptide.

In another embodiment, the invention provides isolated TASK polypeptide encoded by any of the isolated nucleic acid sequences hereinabove identified.

In a certain aspect, the invention concerns an isolated TASK polypeptide, comprising an amino acid sequence having at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a TASK polypeptide having a full-length amino acid sequence as disclosed herein, or an amino acid sequence encoded by any of the nucleic acid sequences disclosed herein or any other specifically defined fragment of a full-length TASK polypeptide amino acid sequence as disclosed herein.

In a further aspect, the invention concerns an isolated TASK polypeptide comprising an amino acid sequence having at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to an amino acid sequence encoded by any of the human protein cDNAs as disclosed herein.

Another aspect of the invention provides an isolated TASK polypeptide. Processes for producing the same are also herein described, wherein those processes comprise culturing a host cell comprising a vector which comprises the appropriate encoding nucleic acid molecule under conditions suitable for expression of the TASK polypeptide and recovering the TASK polypeptide from the cell culture.

In other embodiments of the present invention, the invention provides vectors comprising DNA encoding any of the herein described polypeptides. Host cell comprising any such vector are also provided. By way of example, the host cells may be mammalian cells, E. coli, or yeast.

In a still further embodiment, the invention concerns a composition of matter comprising a TASK nucleic acid as described herein, a chimeric TASK nucleic acid as described herein, or a TASK double-stranded RNA complex.

In yet another embodiment, the invention concerns an article of manufacture comprising a container and a composition of matter contained within the container, wherein the composition of matter may comprise (a) a TASK nucleic acid as described herein, (b) a chimeric TASK nucleic acid as described herein, or (c) a TASK double-stranded RNA complex and a vector capable of delivering (a), (b) or (c). The article may further optionally comprise a label affixed to the container, or a package insert included with the container, that refers to the use of the composition of matter for the therapeutic treatment or diagnostic detection of a tumor.

Another embodiment of the present invention is directed to the use of (a) a TASK nucleic acid as described herein, (b) a chimeric TASK nucleic acid as described herein, (c) a TASK double-stranded RNA complex and a vector capable of delivering (a), (b), or (c), for the preparation of a medicament useful in the treatment of a condition which is responsive to the TASK nucleic acid a chimeric TASK nucleic acid, TASK double-stranded RNA complex and a vector capable of delivering (a), (b), or (c).

Another embodiment of the present invention is directed to a method for killing a cancer cell that expresses a TASK polypeptide, wherein the method comprises contacting the cancer cell with a double-stranded RNA complex that binds to the TASK nucleic acid, and resulting in the death of the cancer cell Another embodiment of the present invention is directed to a method for inhibiting the growth of a cancer cell, wherein the growth of said cancer cell is at least in part dependent upon the growth potentiating effect(s) of a TASK polypeptide, wherein the method comprises contacting the TASK nucleic acid with double-stranded RNA complex that binds to the TASK nucleic acid, thereby antagonizing the growth-potentiating activity of the TASK polypeptide and, in turn, inhibiting the growth of the cancer cell. Preferably the growth of the cancer cell is completely inhibited.

Yet another embodiment of the present invention is directed to a method of therapeutically treating a TASK polypeptide-expressing tumor in a mammal, wherein the method comprises administering to the mammal a therapeutically effective amount of double-stranded RNA complex that binds to the TASK nucleic acid, thereby resulting in the effective therapeutic treatment of the tumor.

Yet another embodiment of the present invention is directed to a method of therapeutically treating a tumor in a mammal, wherein the growth of said tumor is at least in part dependent upon the growth potentiating effect(s) of a TASK polypeptide, wherein the method comprises administering to the mammal a therapeutically effective amount of double-stranded RNA complex that binds to the TASK nucleic acid, thereby antagonizing the growth potentiating activity of said TASK polypeptide and resulting in the effective therapeutic treatment of the tumor.

Yet another embodiment of the present invention is directed to a method for treating or preventing a cell proliferative disorder associated with altered, preferably increased, expression or activity of a TASK polypeptide, the method comprising administering to a subject in need of such treatment an effective amount of an antagonist of a TASK polypeptide. Preferably, the cell proliferative disorder is cancer and the antagonist of the TASK polypeptide is a TASK double-stranded RNA complex. Effective treatment or prevention of the cell proliferative disorder may be a result of direct killing or growth inhibition of cells that express a TASK polypeptide or by antagonizing the cell proliferative activity of a TASK polypeptide.

Yet another embodiment of the present invention is directed to a method of determining the presence of a TASK nucleic acid in a sample suspected of containing the TASK nucleic acid, wherein the method comprises exposing the sample to double-stranded RNA complex that binds to the TASK nucleic acid and determining binding of the double-stranded RNA complex to the TASK nucleic acid in the sample, wherein the presence of such binding is indicative of the presence of the TASK polypeptide in the sample. Optionally, the sample may contain cells (which may be cancer cells) suspected of expressing the TASK polypeptide. The TASK binding double-stranded RNA complex employed in the method may optionally be detectably labeled, attached to a solid support, or the like.

A further embodiment of the present invention is directed to a method of diagnosing the presence of a tumor in a mammal, wherein the method comprises detecting the level of expression of a gene encoding a TASK polypeptide (a) in a test sample of tissue cells obtained from said mammal, and (b) in a control sample of known normal cells of the same tissue origin, wherein a higher level of expression of the TASK polypeptide in the test sample, as compared to the control sample, is indicative of the presence of tumor in the mammal from which the test sample was obtained.

Another embodiment of the present invention is directed to a method of diagnosing the presence of a tumor in a mammal, wherein the method comprises (a) contacting a test sample comprising tissue cells obtained from the mammal with double-stranded RNA complex that binds to a TASK nucleic acid and (b) detecting the formation of a complex between the double-stranded RNA complex and the TASK nucleic acid in the test sample, wherein the formation of a complex is indicative of the presence of a tumor in the mammal. Optionally, the double-stranded RNA complex employed is detectably labeled, attached to a solid support, or the like, and/or the test sample of tissue cells is obtained from an individual suspected of having a cancerous tumor.

Another embodiment of the present invention is directed to an isolated double-stranded RNA complex comprising two RNA strands of 10 to 50 nucleotides in length, wherein portions of the first strand is sufficiently complementary (e.g. having at least 80% identity) to a TASK nucleic acid of interest, where in the double-stranded RNA complex is capable of suppressing, ameliorating, reducing, or “knocking-down” the expression of the TASK polypeptide.

### BRIEF DESCRIPTION OF THE DRAWINGS

FIG. 1 shows a diagram of an exemplary embodiment of a retroviral vector suitable for inducible expression of a RNA coding region. The retroviral vector comprises a 5′ retroviral LTR, a gateway site (“attR1) a modified polIII promoter that comprises two tet operon sites, driving expression of a sequence encoding a shRNA sequence, a second gateway site (attR2), a pol II promoter (the human beta-actin promoter) driving expression of codon optimized TetR sequence and the coding region for the puromycin selectable marker, and a 3′ retroviral LTR. Included in the vector are a 5′HTLV intron and an IRES sequence. Not pictured: a RNA termination sequence is added at the end of the shRNA encoding sequence.

FIG. 2: (A) Codon optimization of TetR open reading frame (ORF) increased translation of the tet repressor protein. Both the original (WT) and codon-optimized (OPT) TetR ORFs were transiently expressed in 293T cells. Forty-eight hours post-transfection, cell lysates were prepared and Western Blotted with an anti-TetR antibody. (B): Maximizing TetR expression prevents unwanted silencing of target genes in the absence of Dox. Cells (293HEK) stably expressing a GFP-MELK fusion construct were transfected with the following pHUSH vector series containing a Melk targeting shRNA: pHUSH lacking an H1-shRNA cassette (empty vector, lane 1); pHUSH (lane 2); pHUSH^{-IVS} (removal of the synthetic intron sequence between the TetR ORF and the IRES, lane 3); pHUSH^{-IVS}TetR^{Opt} (replacement of the TetR in pHUSH^{-IVS} with a codon-optimized TetR, lane 4). Stable pools of cells were selected by culturing in the presence of 3 μg/ml puromycin. The resulting stable pools were treated in the absence (open bars) or presence (filled bars) of 1 μg/ml doxycycline for five days and the level of GFP-Melk expression analyzed by FACS. Data is normalized to the mean fluorescence intensity of cells containing the pHUSH empty vector (“EV”) control (lane 1). Placement of two Tet02 operons within the H1 promoter increases sensitivity to low levels of TetR. Cells were transfected with a pGL3-huMelk in the presence of increasing concentrations (1:0, 1:2, and 1:10 molar ratio) of codon optimized TetR.

FIG. 3: Optimized selection pressures improves H1-shRNA mediated knockdown in LOX-IMV1 melanoma cells. By enhancing selection pressure with increasing amounts of puromycin (2 ug/mL to 5 ug/mL), robust B-Raf knockdown was observed by qRT-PCR. Stables pools at 2 ug/mL puromycin (A) were compared to the average knockdown of 54 clones derived from a pool selected at 5 ug/mL puromycin (B). Cells were treated ±Dox for 72 hours. Dark grey bars indicate only three clones with less than 50% knockdown upon DOX addition. The level of b-raf knockdown was determined by qRT-PCR and data normalized by the 2̂ddCT method where 1 represents the level of b-raf in empty vector control cells. Dotted lines represent average knockdown (43% knockdown in A, and 73% knockdown in B).

FIG. 4: Dose response of doxycycline-induced gene knock-down in vitro and in vivo. Regulated shRNA-mediated depletion of luciferase in a pHUSH shGL3, luciferase expressing SVT2 clone in vitro. Cells were cultured for 2 (diamonds), 4 (squares) or 7 days (triangles) at the indicated dose of doxycycline and then harvested to determine luciferase expression.

FIG. 5: Effect of Doxycycline dose on luciferase knock-down in an in vivo tumor growth assay. A pHUSH shGL3 clone (clone B11) was injected s.c. as described in the Examples. Ten days post injection, animals were administered sucrose water alone (animals 1A and 1B), sucrose with doxycycline at 1 mg/ml (animals 2A and 2B), 0.1 mg/ml (animals 3A and 3B) or 0.01 mg/ml (animals 4A and 4B). Representative images for two animals within each treatment arm at 0, 3 and 6 days post doxycycline addition to drinking water.

FIG. 6: (A) The relative change in mean bioluminescence at each time point and treatment condition (n=5 mice per doxycycline dose). (B) Doxycycline or shRNA expression per se does not influence tumor growth in vivo. In addition to the luciferase expression analysis, tumor volume measurements were recorded for the pHUSH shGL3 clone B11 at each concentration of doxycycline.

FIG. 7: (A) Diagram of an exemplary embodiment of an inducible vector for transgenic delivery of a RNA coding region. The vector comprises a gateway site (“attR1) a modified polIII promoter that comprises a tet operon site driving expression of a sequence encoding a shRNA sequence, a second gateway site (attR2), a pol II promoter (the human beta-actin promoter) driving expression of codon optimized TetR sequence and the coding region for the puromycin selectable marker. Also included in the vector are a 5′HTLV intron and an IRES sequence. (B) Mouse embryonic stem cell (ES) clones containing sh-muMelk-B pHUSH were electroporated and selected in puromycin (1.0-1.5 μg/mL) as described in the Examples. Individual clones were picked and those with the highest level of TetR expression were assessed for Melk knockdown. Shown are three clones (A6, D2 and D3) with 90-95% Melk knockdown upon doxycycline treatment (three days at 1 μg/ml doxycycline. (C) Vector-mediated knock-down of murine Melk caused a delay in cardiomyocyte formation. The ability of ES sh-muMelk-B pHUSH clones (A6, D2, D3) or parental ES cells (R1) to form cardiomyocytes ±1 μg/ml doxycycline was assessed. Individual colonies (25 per clone) were scored visually for the presence or absence of observable cardiomyocyte morphology. The data was normalized as the percent of colonies that formed cardiomyocytes.

FIG. 8: Melk knockdown is maintained in differentiated ES cells. The ES sh-muMelk-B-pHUSH clones described in the above panels, were allowed to differentiate into embryoid bodies in the presence or absence of 1 μg/ml doxycycline and the level of Melk expression determined by qRT-PCR.

FIG. 9: Insertion of Pol II expression cassettes into an inducible expression vector (proEX). (A) Schematic representation of an exemplary embodiment of a vector enabling regulated GFP expression. The vector comprises a 5′ retroviral LTR, a gateway site (“attR1) a modified polII promoter (CMV) that comprises a tet operon site, driving expression of a sequence encoding eGFP, a second gateway site (attRz), a pol II promoter (the human beta-actin promoter) driving expression of codon optimized TetR sequence and the coding region for the puromycin selectable marker, and a 3′ retroviral LTR. Included in the vector are a 5′HTLV intron and an IRES sequence. A shuttle vector containing a modified CMV promoter with the Tet-operator (CMV-TO), was generated as described in the Examples, and transferred into an acceptor plasmid (pHUSH-GW) by Gateway™ cloning reaction. (B) Regulated expression of GFP with proEX mediated delivery. HCT116 cells were transduced with the proEX-GFP virus, selected with puromycin and GFP expression assessed by FACS after incubation for 48 hours in the presence or absence of 1 ug/ml doxycycline.

FIG. 10: Inducible knockdown of BRAF expression prevents melanoma tumor growth. (A). Schematic representation of a retroviral vector for tetracycline/doxycycline-inducible synthesis of shRNA. The Tet-repressor is constitutively expressed from the β-actin promoter and H1 RNA polymerase III promoter-driven transcription is thereby repressed in the absence of Dox by binding of the TetR protein to the Tet-responsive element sequence located immediately downstream of the TATA box. Addition of Dox results in dissociation of the TetR protein and derepression of the shRNA transcriptional unit. TATA, TATA box; TRE, Tet-responsive element; TetR, Tet-repressor protein; IRES, internal ribosomal entry site; PURO, puromycin resistance gene; LTR, long terminal repeat. B. Experimental validation of BRAF knockdown in melanoma cell lines. LOX-IMVI and A375 cell clones stably expressing BRAF shRNA or control GFP and Luciferase (Luc) shRNAs were treated with the indicated Dox concentrations for 72 h. Lysates were then analyzed by immunoblotting. (C-E) BRAF shRNA knockdown demonstrates anti-tumor efficacy in xenograft models. LOX-IMVI and A375 inducible shRNA cells were implanted subcutaneously in the flank of athymic mice as described in the Materials and Methods. Treatment in each experiment was initiated on the day when mice had tumors ranging in size from 100 to 150 mm³. Administration of 2 mg/ml Dox via drinking water produced regression in (C) LOX-IMVI or stasis in (D) A375 tumors expressing an inducible BRAF-specific shRNA. (E) GFP or (C) Luciferase control shRNAs did not affect tumor growth kinetics. No lethality or weight loss was observed.

FIG. 11: BRAF knockdown is reversible and tightly regulated in vivo. (A) BRAF-dependent LOX-IMVI tumors were allowed to grow for 14 days before administration of Dox was initiated to knockdown BRAF-dependent signaling and tumorigenesis. (B) Dox-treated mice with regressing subcutaneous tumors that are subsequently removed from Dox at day 14 undergo tumor recurrence. (C) Dose response of in vivo BRAF knockdown. Groups of mice with subcutaneous LOX-IMVI/BRAF-shRNA tumors of equivalent size were induced with 0, 0.02, 0.2, 0.5, 1.0 or 2.0 mg/ml Dox and 5% sucrose as indicated. Tumor volumes (mm³) are presented as mean ±SD.

FIG. 12: Conditional knockdown of BRAF using a second shRNA induces tumor regression. (A) Western analysis of two LOX-IMVI/BRAF-shRNA2 cell clones for changes in BRAF protein expression and ERK1/2 phosphorylation upon 2 μg/ml Dox treatment. (B) LOX-IMVI/BRAF-shRNA2 xenograft mice exhibit tumor regression upon 1 mg/ml Dox treatment. Each cohort consisted of ten mice.

FIG. 13: Histological analysis of regressing tumors. LOX-IMVI/BRAF-shRNA tumor-bearing mice were placed on 1 mg/ml Dox and sacrificed as indicated. Tumor tissue was analyzed by immunochemistry using antibodies specific for Ki-67, cleaved caspase-3 or MECA-32 (brown staining). No staining was observed in the naive IgG control.

FIG. 14: Reduction of A375M systemic tumor growth by BRAF shRNA knockdown. (A) Western blot analysis showing expression of BRAF and phosphorylation of MEK1 in uninduced cells (lane 1) and cells treated with 2 mg/ml Dox for 72 h (lane 2). Total MEK1 serves as an internal control to show equal loading. (B) Kaplan-Meier survival data of scid-beige mice intravenously injected with 4×10⁵ A375M-luc/shRNA-BRAF cells and receiving drinking water containing 5% sucrose only (control) or sucrose with 1 mg/ml Dox. Animals were monitored for tumor onset and illness until they reached a terminal stage and were euthanized. Each group consisted of at least 10 mice. The reduction in tumor growth conferred by Dox-mediated BRAF knockdown is significant according to the log-rank test, p<0.0001. Median survival of all mice treated with 1 mg/ml Dox was 33.4 days, in comparison to 27.6 days for control mice. (C) Representative in vivo bioluminescence imaging of visible light emitted upon injection of mice with luciferin and (D) quantification of tumor burden of mice receiving Dox versus sucrose-treated control mice. Homogeneous cohorts of mice with established tumor lesions were divided into treatment groups 2 weeks after injection of A375M-luc/shRNA-BRAF cells. Bioluminescence is represented as relative to the intensity at day 14 for each animal.

FIG. 15: Luciferase-based transient co-transfection experiments compared shRNA and miRNA inducible expression vector performance. A. Comparison of H1-shRNA and CMV-miR vectors corresponding to murine p53 sequences transfected at 7.5:1, 5:1 and 2.5:1 shRNA:pGL3-mu-p53. B. The removal of the EmGFP leader rendered miR-p53-1 non-functional at a co-transfection ratio of 10:1.

FIG. 16: A. Diagram of an exemplary embodiment of a lentiviral vector. The vector comprises a lentiviral 5′LTR, a gateway site (suitable for insertion of an RNA expression cassette via gateway-mediated recombination, as described herein and diagrammed in FIG. 16B), a pol II promoter (the human beta-actin promoter) driving expression of codon optimized TetR sequence and the coding region for the puromycin selectable marker, and a 3′ lentiviral LTR. Included in the vector are a 5′HTLV intron and an IRES sequence. B. A dual-color, inducible lentiviral miRNA expression vector. The vector comprises a lentiviral 5′LTR, a gateway site, a polII promoter (CMV) comprising a tet operon driving expression of a miRNA coding sequence (EmGFP-miR), a gateway site, a pol II promoter (the human beta-actin promoter) driving expression of codon optimized TetR sequence and the coding region for the puromycin selectable marker, and a 3′ lentiviral LTR. Included in the vector are a 5′HTLV intron and an IRES sequence. The expression of the red fluorescent protein dsRED is constitutive and linked to the TetR via and IRES. Expression of the green fluorescent protein EmGFP and the miRNA is induced by culturing in Dox.

FIG. 17 shows the polynucleotide sequence of the optimized TetR (SEQ ID NO:1)

FIG. 18 shows the amino acid sequence of the optimized TetR (SEQ ID NO:2) derived from the coding sequence of SEQ ID NO:3 shown in FIG. 17.

FIG. 19 shows the polynucleotide sequence of the Melk kinase-GFP fusion (SEQ ID NO:3).

FIG. 20 shows the amino acid sequence of the Melk kinase-GFP fusion (SEQ ID NO:4) derived from the coding sequence of SEQ ID NO:5 shown in FIG. 19.

FIG. 21 shows the polynucleotide sequence of the Melk kinase-Luciferase fusion (SEQ ID NO:5)

FIG. 22 shows the amino acid sequence of the Melk kinase-Luciferase fusion (SEQ ID NO:6) derived from the coding sequence of SEQ ID NO:7 shown in FIG. 21.

### DETAILED DESCRIPTION OF THE PREFERRED EMBODIMENTS

### I. Definitions

The terms “TASK polypeptide” and “TASK” as used herein and when immediately followed by a numerical designation, refer to various polypeptides, wherein the complete designation (i.e., TASK/number) refers to specific polypeptide sequences as described herein. The terms “TASK/number polypeptide” and “TASK/number” wherein the term “number” is provided as an actual numerical designation as used herein encompass native sequence polypeptides, polypeptide variants and fragments of native sequence polypeptides and polypeptide variants (which are further defined herein). The TASK polypeptides described herein may be isolated from a variety of sources, such as from human tissue types or from another source, or prepared by recombinant or synthetic methods. The term “TASK polypeptide” refers to each individual TASK/number polypeptide disclosed herein. All disclosures in this specification which refer to the “TASK polypeptide” refer to each of the polypeptides individually as well as jointly. For example, descriptions of the preparation of, purification of, derivation of, formation of antibodies to or against, formation of TASK double-stranded RNA complexes to or against, formation of TASK binding small molecules to or against, administration of, compositions containing, treatment of a disease with, etc., pertain to each polypeptide of the invention individually. The term “TASK polypeptide” also includes variants of the TASK/number polypeptides disclosed herein.

A “native sequence TASK polypeptide” comprises a polypeptide having the same amino acid sequence as the corresponding TASK polypeptide derived from nature. Such native sequence TASK polypeptides can be isolated from nature or can be produced by recombinant or synthetic means. The term “native sequence TASK polypeptide” specifically encompasses naturally-occurring truncated forms of the specific TASK polypeptide, naturally-occurring variant forms (e.g., alternatively spliced forms) and naturally-occurring allelic variants of the polypeptide. In certain embodiments of the invention, the native sequence TASK polypeptides disclosed herein are mature or full-length native sequence polypeptides comprising the full-length amino acids sequences shown in the accompanying figures. Start and stop codons (if indicated) are shown in bold font and underlined in the figures. Nucleic acid residues indicated as “N” in the accompanying figures are any nucleic acid residue.

“Percent (%) amino acid sequence identity” with respect to the TASK polypeptide sequences identified herein is defined as the percentage of amino acid residues in a candidate sequence that are identical with the amino acid residues in the specific TASK polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations using this method, Tables 2 and 3 demonstrate how to calculate the % amino acid sequence identity of the amino acid sequence designated “Comparison Protein” to the amino acid sequence designated “TASK”, wherein “TASK” represents the amino acid sequence of a hypothetical TASK polypeptide of interest, “Comparison Protein” represents the amino acid sequence of a polypeptide against which the “TASK” polypeptide of interest is being compared, and “X, “Y” and “Z” each represent different hypothetical amino acid residues. Unless specifically stated otherwise, all % amino acid sequence identity values used herein are obtained as described in the immediately preceding paragraph using the ALIGN-2 computer program.

“TASK variant polynucleotide” or “TASK variant nucleic acid sequence” means a nucleic acid molecule which encodes a TASK polypeptide, preferably an active TASK polypeptide, as defined herein and which has at least about 80% nucleic acid sequence identity with a nucleotide acid sequence encoding a full-length native sequence TASK polypeptide sequence as disclosed herein, or any other fragment of a full-length TASK polypeptide sequence as disclosed herein (such as those encoded by a nucleic acid that represents only a portion of the complete coding sequence for a full-length TASK polypeptide). Ordinarily, a TASK variant polynucleotide will have at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% nucleic acid sequence identity with a nucleic acid sequence encoding a full-length native sequence TASK polypeptide sequence as disclosed herein, or any other fragment of a full-length TASK polypeptide sequence as disclosed herein. Variants do not encompass the native nucleotide sequence.

Ordinarily, TASK variant polynucleotides are at least about 5 nucleotides in length, alternatively at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length.

“Percent (%) nucleic acid sequence identity” with respect to TASK-encoding nucleic acid sequences identified herein is defined as the percentage of nucleotides in a candidate sequence that are identical with the nucleotides in the TASK nucleic acid sequence of interest, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. For purposes herein, however, % nucleic acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for nucleic acid sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z

where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 4 and 5, demonstrate how to calculate the % nucleic acid sequence identity of the nucleic acid sequence designated “Comparison DNA” to the nucleic acid sequence designated “TASK-DNA”, wherein “TASK-DNA” represents a hypothetical TASK-encoding nucleic acid sequence of interest, “Comparison DNA” represents the nucleotide sequence of a nucleic acid molecule against which the “TASK-DNA” nucleic acid molecule of interest is being compared, and “N”, “L” and “V” each represent different hypothetical nucleotides. Unless specifically stated otherwise, all % nucleic acid sequence identity values used herein are obtained as described in the immediately preceding paragraph using the ALIGN-2 computer program.

In other embodiments, TASK variant polynucleotides are nucleic acid molecules that encode a TASK polypeptide and which are capable of hybridizing, preferably under stringent hybridization and wash conditions, to nucleotide sequences encoding a full-length TASK polypeptide as disclosed herein. TASK variant polypeptides may be those that are encoded by a TASK variant polynucleotide.

“Isolated,” when used to describe the various TASK polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the TASK polypeptide natural environment will not be present. Ordinarily, however, isolated polypeptide will be prepared by at least one purification step.

An “isolated” TASK polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate), followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

“Treating” or “treatment” or “alleviation” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for a TASK polypeptide-expressing cancer if, after receiving a therapeutic amount of a double-stranded RNA complex according to the methods of the present invention, the patient shows observable and/or measurable reduction in or absence of one or more of the following: reduction in the number of cancer cells or absence of the cancer cells; reduction in the tumor size; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues. To the extent the double-stranded RNA complex may prevent growth and/or kill existing cancer cells, it may be cytostatic and/or cytotoxic. Reduction of these signs or symptoms may also be felt by the patient.

The above parameters for assessing successful treatment and improvement in the disease are readily measurable by routine procedures familiar to a physician. For cancer therapy, efficacy can be measured, for example, by assessing the time to disease progression (TTP) and/or determining the response rate (RR). Metastasis can be determined by staging tests and by bone scan and tests for calcium level and other enzymes to determine spread to the bone. CT scans can also be done to look for spread to the pelvis and lymph nodes in the area. Chest X-rays and measurement of liver enzyme levels by known methods are used to look for metastasis to the lungs and liver, respectively. Other routine methods for monitoring the disease include transrectal ultrasonography (TRUS) and transrectal needle biopsy (TRNB).

For bladder cancer, which is a more localized cancer, methods to determine progress of disease include urinary cytologic evaluation by cystoscopy, monitoring for presence of blood in the urine, visualization of the urothelial tract by sonography or an intravenous pyelogram, computed tomography (CT) and magnetic resonance imaging (MRI). The presence of distant metastases can be assessed by CT of the abdomen, chest x-rays, or radionuclide imaging of the skeleton.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Mammal” refers to any animal classified as a mammal, including humans, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

By “solid phase” or “solid support” is meant a non-aqueous matrix to which double-stranded RNA complex of the present invention can adhere or attach. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of double-stranded RNA complex to a mammal. The components of the liposome are commonly arranged in a bilayer formation, similar to the lipid arrangement of biological membranes.

A “small” molecule or “small” organic molecule is defined herein to have a molecular weight below about 500 Daltons.

The term “transgenic” is used herein to describe the property of harboring a transgene. For instance, a “transgenic organism” is any animal, including mammals, fish, birds and amphibians, in which one or more of the cells of the animal contain nucleic acid introduced by way of human intervention, such as by the methods described herein. In a transgenic animal that comprises a transgene that encodes a gene of interest, the transgene typically causes the cell to express or overexpress a recombinant protein. However, according to some embodiments of the invention, expression of an RNA coding region can be used to down regulate the expression of a particular gene through antisense or RNA interference mechanisms.

“Lentivirus” refers to a genus of retroviruses that are capable of infecting dividing and non-dividing cells. Several examples of lentiviruses include HIV (human immunodeficiency virus; including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates.

A lentiviral genome is generally organized into a 5′ long terminal repeat (LTR), the gag gene, the pol gene, the env gene, the accessory genes (nef, vif, vpr, vpu) and a 3′ LTR. The viral LTR is divided into three regions called U3, R and U5. The U3 region contains the enhancer and promoter elements. The U5 region contains the polyadenylation signals. The R (repeat) region separates the U3 and U5 regions and transcribed sequences of the R region appear at both the 5′ and 3′ ends of the viral RNA. See, for example, “RNA Viruses: A Practical Approach” (Alan J. Cann, Ed., Oxford University Press, (2000)), 0 Narayan and Clements J. Gen. Virology 70:1617-1639 (1989), Fields et al. Fundamental Virology Raven Press. (1990), Miyoshi H, Blomer U, Takahashi M, Gage F H, Verna I M. J. Virol. 72(10):8150-7 (1998), and U.S. Pat. No. 6,013,516.

Lentiviral vectors are known in the art, including several that have been used to transfect hematopoietic stem cells. Such vectors can be found, for example, in the following publications, which are incorporated herein by reference: Evans J T et al. Hum Gene Ther 1999; 10:1479-1489; Case S S, Price M A, Jordan C T et al. Proc Natl Acad Sci USA 1999; 96:2988-2993; Uchida N, Sutton R E, Friera A M et al. Proc Natl Acad Sci USA 1998; 95:11939-11944; Miyoshi H, Smith K A, Mosier D E et al. Science 1999; 283:682-686; Sutton R E, Wu H T, Rigg R et al. Human immunodeficiency virus type 1 vectors efficiently transduce human hematopoietic stem cells. J Virol 1998; 72:5781-5788.

“Virion,” “viral particle” and “retroviral particle” are used herein to refer to a single virus comprising an RNA genome, pol gene derived proteins, gag gene derived proteins and a lipid bilayer displaying an envelope (glyco)protein. The RNA genome is usually a recombinant RNA genome and thus may contain an RNA sequence that is exogenous to the native viral genome. The RNA genome may also comprise a defective endogenous viral sequence.

A “pseudotyped” retrovirus is a retroviral particle having an envelope protein that is from a virus other than the virus from which the RNA genome is derived. The envelope protein may be from a different retrovirus or from a non-retroviral virus. A preferred envelope protein is the vesicular stomatitus virus G (VSV G) protein. However, to eliminate the possibility of human infection, viruses can alternatively be pseudotyped with ecotropic envelope protein that limit infection to a specific species, such as mice or birds. For example, in one embodiment, a mutant ecotropic envelope protein is used, such as the ecotropic envelope protein 4.17 (Powell et al. Nature Biotechnology 18(12):1279-1282 (2000)).

The term “provirus” is used to refer to a duplex DNA sequence present in a eukaryotic chromosome that corresponds to the genome of an RNA retrovirus. The provirus may be transmitted from one cell generation to the next without causing lysis or destruction of the host cell.

A “self-inactivating 3′LTR” is a 3′ long terminal repeat (LTR) that contains a mutation, substitution or deletion that prevents the LTR sequences from driving expression of a downstream gene. A copy of the U3 region from the 3′ LTR acts as a template for the generation of both LTR's in the integrated provirus. Thus, when the 3′ LTR with an inactivating deletion or mutation integrates as the 5′ LTR of the provirus, no transcription from the 5′ LTR is possible. This eliminates competition between the viral enhancer/promoter and any internal enhancer/promoter. Self-inactivating 3′ LTRs are described, for example, in Zufferey et al. J. Virol. 72:9873-9880 (1998), Miyoshi et al. J. Virol. 72:8150-8157 and Iwakuma et al. Virology 261:120-132 (1999).

An “effective amount” of double-stranded RNA complex as disclosed herein is an amount sufficient to carry out a specifically stated purpose. An “effective amount” may be determined empirically and in a routine manner, in relation to the stated purpose by means of titration.

The term “therapeutically effective amount” refers to an amount of double-stranded RNA complex effective to “treat” a disease or disorder in a subject or mammal. In the case of cancer, the therapeutically effective amount of the double-stranded RNA complex may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See the definition herein of “treating”. To the extent the drug may prevent growth and/or kill existing cancer cells, it may be cytostatic and/or cytotoxic.

A “growth inhibitory amount” of double-stranded RNA complex is an amount capable of inhibiting the growth of a cell, especially tumor, e.g., cancer cell, either in vitro or in vivo. A “growth inhibitory amount” of double-stranded RNA complex for purposes of inhibiting neoplastic cell growth may be determined empirically and in a routine manner.

A “cytotoxic amount” of double-stranded RNA complex is an amount capable of causing the destruction of a cell, especially tumor, e.g., cancer cell, either in vitro or in vivo. A “cytotoxic amount” of a double-stranded RNA complex for purposes of inhibiting neoplastic cell growth may be determined empirically and in a routine manner.

An “RNA coding region” is a nucleic acid that can serve as a template for the synthesis of an RNA molecule, such as a double-stranded RNA complex. Preferably, the RNA coding region is a DNA sequence.

The term “RNA interference or silencing” is broadly defined and includes all posttranscriptional and transcriptional mechanisms of RNA mediated inhibition of gene expression, such as those described in (P. D. Zamore Science 296, 1265 (2002)).

A “TASK interfering RNA” or “TASK double-stranded RNA complex” binds, preferably specifically, to a TASK nucleic acid and reduces its expression. This means the expression of the TASK molecule is lower with the double-stranded RNA complex present as compared to expression of the TASK molecule in a control where the double-stranded RNA complex is not present. TASK double-stranded RNA complex may be identified and synthesized using known methods (Shi Y., Trends in Genetics 19(1):9-12 (2003), WO/2003056012 and WO2003064621).

A double-stranded RNA complex “which binds” a nucleic acid of interest, e.g. a nucleic acid encoding a TASK polypeptide target, is one that binds the target sequence with sufficient affinity such that the double-stranded RNA complex is useful as a diagnostic and/or therapeutic agent in targeting a cell or tissue expressing the antigen. In such embodiments, the extent of binding of the double-stranded RNA complex to a “non-target” sequence will be less than about 10% of the binding of the double-stranded RNA complex to its particular target protein as determined by hybridization. With regard to the binding of double-stranded RNA complex, the term “specific binding” or “specifically binds to” or is “specific for” a particular nucleic acid means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target.

An double-stranded RNA complex or other organic molecule that “inhibits the growth of tumor cells expressing a TASK polypeptide” or a “growth inhibitory” oligopeptide, double-stranded RNA complex or other organic molecule is one which results in measurable growth inhibition of cancer cells expressing or overexpressing the appropriate TASK polypeptide. Preferred growth inhibitory anti-TASK antibodies, oligopeptides, double-stranded RNA complex or organic molecules inhibit growth of TASK-expressing tumor cells by greater than 20%, preferably from about 20% to about 50%, and even more preferably, by greater than 50% (e.g., from about 50% to about 100%) as compared to the appropriate control, the control typically being tumor cells not treated with the oligopeptide, double-stranded RNA complex or other organic molecule being tested. Growth inhibition of tumor cells in vivo can be determined in various ways such as is described in the Experimental Examples section below.

An oligopeptide, double-stranded RNA complex or other organic molecule which “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies). The cell is usually one that overexpresses a TASK polypeptide. Preferably the cell is a tumor cell, e.g., a prostate, breast, ovarian, stomach, endometrial, lung, kidney, colon, or bladder cell. Various methods are available for evaluating the cellular events associated with apoptosis. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells. Preferably, the double-stranded RNA complex or other organic molecule which induces apoptosis is one which results in about 2 to 50 fold, preferably about 5 to 50 fold, and most preferably about 10 to 50 fold, induction of annexin binding relative to untreated cell in an annexin binding assay.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, multiple myeloma and B-cell lymphoma, brain, as well as head and neck cancer, and associated metastases.

The terms “cell proliferative disorder” and “proliferative disorder” refer to disorders that are associated with some degree of abnormal cell proliferation. In one embodiment, the cell proliferative disorder is cancer.

“Tumor”, as used herein, refers to all neoplastic cell growth and proliferation, whether malignant or benign, and all pre-cancerous and cancerous cells and tissues.

A double-stranded RNA complex, oligopeptide or other small molecule which “induces cell death” is one which causes a viable cell to become nonviable. The cell is one which expresses a TASK polypeptide, preferably a cell that overexpresses a TASK polypeptide as compared to a normal cell of the same tissue type. Preferably, the cell is a cancer cell, e.g., a melanoma, breast, ovarian, stomach, endometrial, salivary gland, lung, kidney, colon, thyroid, pancreatic or bladder cell. Cell death in vitro may be determined in the absence of complement and immune effector cells to distinguish cell death induced by antibody-dependent cell-mediated cytotoxicity (ADCC) or complement dependent cytotoxicity (CDC). Thus, the assay for cell death may be performed using heat inactivated serum (i.e., in the absence of complement) and in the absence of immune effector cells. To determine whether the oligopeptide, double-stranded RNA complex or other small molecule is able to induce cell death, loss of membrane integrity as evaluated by uptake of propidium iodide (PI), trypan blue (see Moore et al. Cytotechnology 17:1-11 (1995)) or 7AAD can be assessed relative to untreated cells. Preferred cell death-inducing double-stranded RNA complex, oligopeptides or other small molecules are those which induce PI uptake in the PI uptake assay in BT474 cells.

A “TASK-expressing cell” is a cell which expresses an endogenous or transfected TASK polypeptide. A “TASK-expressing cancer” is a cancer comprising cells that overexpress a TASK polypeptide. A “TASK-expressing cancer” optionally produces sufficient levels of TASK polypeptide, such that a double-stranded RNA complex, oligopeptide or other small molecule can bind thereto and have a therapeutic effect with respect to the cancer. In another embodiment, a “TASK-expressing cancer” optionally expresses sufficient levels of the TASK gene, such that a TASK double-stranded RNA complex can bind thereto and have a therapeutic effect with respect to the cancer. A cancer which “overexpresses” a TASK polypeptide is one which has significantly higher levels of TASK polypeptide thereof, compared to a noncancerous cell of the same tissue type. Such overexpression may be caused by gene amplification or by increased transcription or translation. TASK polypeptide overexpression may be determined in a diagnostic or prognostic assay by evaluating increased levels of the TASK protein present in the cell (e.g., via an immunohistochemistry assay using anti-TASK antibodies prepared against an isolated TASK polypeptide which may be prepared using recombinant DNA technology from an isolated nucleic acid encoding the TASK polypeptide; FACS analysis, etc.). Alternatively, or additionally, one may measure levels of TASK polypeptide-encoding nucleic acid or mRNA in the cell, e.g., via fluorescent in situ hybridization using a nucleic acid based probe corresponding to a TASK-encoding nucleic acid or the complement thereof; (FISH; see WO98/45479 published October, 1998), Southern blotting, Northern blotting, or polymerase chain reaction (PCR) techniques, such as real time quantitative PCR (qRT-PCR). Aside from the above assays, various in vivo assays are available to the skilled practitioner. For example, one may expose cells within the body of the patient to an antibody which is optionally labeled with a detectable label, e.g., a radioactive isotope, and binding of the antibody to cells in the patient can be evaluated, e.g., by external scanning for radioactivity or by analyzing a biopsy taken from a patient previously exposed to the antibody.

The word “label” when used herein refers to a detectable compound or composition which is conjugated directly or indirectly to the antibody, oligopeptide or other small molecule so as to generate a “labeled” antibody, oligopeptide or other small molecule. The label may be detectable by itself (e.g. radioisotope labels or fluorescent labels) or, in the case of an enzymatic label, may catalyze chemical alteration of a substrate compound or composition which is detectable.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “growth inhibitory agent” when used herein refers to a compound or composition which inhibits growth of a cell, especially a TASK-expressing cancer cell, either in vitro or in vivo. Thus, the growth inhibitory agent may be one which significantly reduces the percentage of TASK-expressing cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and: Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

“Doxycycline” is a member of the tetracycline family of antibiotics. The full chemical name of doxcycline is 1-dimethylamino-2,4-a,5,7,12-pentahydroxy-11-methyl-4,6-dioxo-1,4a,11,11a,12,12a-hexahydrotetracene-3-carboxamide. Doxycycline will bind the TetR and relieve the TetR inhibition of the TetO.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

The term “package insert” is used to refer to instructions customarily included in commercial packages of therapeutic products, that contain information about the indications, usage, dosage, administration, contraindications and/or warnings concerning the use of such therapeutic products.

A “polynucleotide” (interchangeably termed “nucleic acid”) as used herein, refers to a non-naturally occurring, recombinantly produced, polymeric form of nucleotides of any length, either ribonucleotides or deoxyribonucleotides, or analogs thereof. This term refers to the primary structure of the molecule, and thus includes double- and single stranded DNA, as well as double- and single-stranded RNA. It also includes modified polynucleotides such as methylated and/or capped polynucleotides. The polynucleotide can either be an isolate, or integrated in another nucleic acid molecule e.g. in an expression vector or the chromosome of an eukaryotic host cell. Polynucleotide includes self-replicating plasmids. The terms “construct” and “vector” are used interchangeably with “polynucleotide” herein. Vector includes shuttle and expression vectors. Typically, the plasmid construct will also include an origin of replication (e.g., the ColE1 origin of replication) and a selectable marker (e.g., ampicillin or tetracycline resistance), for replication and selection, respectively, of the plasmids in bacteria. A polynucleotide or construct includes but does not have to be, an expression vector. An “expression vector” refers to a construct that contains the necessary regulatory elements for expression of at least the amplifiable selectable gene, GFP gene and selected sequence in the host cell.

A “selectable marker gene” or “marker gene” is a gene that allows cells carrying the gene to be specifically selected for or against. In some embodiments, the selection is in the presence of a selection agent. In some embodiments (such as embodiments using a fluorescent protein as a marker), the selection process uses detection of fluorescence. By way of illustration, an antibiotic resistance gene can be used as a positive selectable marker gene that allows the host cell transformed with the gene to be positively selected for in the presence of the corresponding antibiotic; a non-transformed host cell would not be capable of growth or survival under the selection culture conditions. Selectable markers can be positive, negative or bifunctional. Positive selectable markers allow selection for cells carrying the marker, whereas negative selection markers allow cells carrying the marker to be selectively eliminated. Typically, a selectable marker gene will confer resistance to a drug or compensate for a metabolic or catabolic defect in the host cell. The selectable marker genes used herein including the amplifiable selectable genes, will include variants, fragments, functional equivalents, derivatives, homologs and fusions of the native selectable marker gene so long as the encoded product retains the selectable property. Useful derivatives generally have substantial sequence similarity (at the amino acid level) in regions or domains of the selectable marker associated with the selectable property. A variety of marker genes have been described, including bifunctional (i.e., positive/negative) markers (see e.g., WO 92/08796, published 29 May 1992, and WO 94/28143, published 8 Dec. 1994), incorporated by reference herein. For example, selectable genes commonly used with eukaryotic cells include the genes for aminoglycoside phosphotransferase (APH), hygromycin phosphotransferase (hyg), dihydrofolate reductase (DHFR), thymidine kinase (tk), glutamine synthetase, asparagine synthetase, and genes encoding resistance to neomycin (G418), puromycin, histidinol D, bleomycin and phleomycin. Fluorescent proteins, such as GFP and dsRed, are also commonly used as markers.

As used herein, a “fluorescent protein” refers to any protein that emits sufficient fluorescence to enable fluorescence detection of the protein intracellularly by, e.g., fluorescence microscopy or flow cytometry. Preferably, host cells expressing fluorescent proteins can be detected using a fluorescence-activated cell sorter (FACS). Examples of fluorescent proteins include green, cyan, blue, yellow as well as other fluorescent proteins from the coelenterate sub-phylum Cnidaria. The fluorescent protein encoding sequences can be native (wild-type) genes, or variants of the genes which are synthetic prepared such as by genetic engineering. A preferred fluorescent protein is green fluorescent protein (GFP), preferably from Aequorea victoria. In one embodiment, the Aequorea GFP mutant, S65T, (described below) is used.

Two well characterized GFPs are from the jellyfish, Aequorea victoria, and a sea pansy, Renilla reniformis. Aequorea and Renilla GFPs each transmute blue chemiluminescence from a distinct primary photoprotein into green fluorescence. Aequorea GFP is a protein of 238 amino acid residues. The protein is maximally excited with blue light with a bigger absorbance peak at 395 nm and a smaller peak at 475 nm, and emits green light at 508-509 nm. The mature purified protein is highly stable, remaining fluorescent up to 65° C., pH11, 1% SDS or 6M guanidinum chloride, and resisting most proteases for may hours. Renilla GFP is an even more stable protein than Aequorea GFP; it shows a single absorption peak at 498 nm with an emission peak at 509 nm. For a review of the properties of Aequorea and Renilla GFPs, see, e.g., Chalfie et al., Science 263: 802-805 (1994); and Cubitt et al, Trends Biochem. Sci. 20: 448-455 (1995). GFP can fluoresce in both transformed prokaryotic and eukaryotic cells.

“Selected sequence” or “product gene” or “gene of interest” have the same meaning herein and refer to a polynucleotide sequence of any length that encodes a product of interest. The selected sequence can be a full length or a truncated gene, a fusion or tagged gene, and can be a cDNA, a genomic DNA, or a DNA fragment, preferably, a cDNA. The selected sequence can be the native sequence i.e., naturally occurring form(s), or can be mutated or otherwise modified as desired. These modifications include humanization, codon replacement to optimize codon usage in the selected host cell or tagging. The selected sequence can encode a secreted, cytoplasmic, nuclear, membrane bound or cell surface polypeptide. Expression of the selected sequence should not be detrimental to the host cell or compromise cell viability. The “desired product” includes proteins, polypeptides and fragments thereof, peptides, and double-stranded RNA complexes, which are capable of being expressed in the selected eukaryotic host cell. The proteins can be hormones, cytokines and lymphokines, antibodies, receptors, adhesion molecules, enzymes, and fragments thereof. The desired proteins can serve as agonist or antagonist, and/or have therapeutic or diagnostic uses. The present polynucleotides are most suitable for expression of desired products of mammalian origin although microbial and yeast products can also be produced.

The terms “polypeptide” and “protein” are used interchangeably to refer to polymers of amino acids of any length. These terms also include proteins that are post-translationally modified through reactions that include glycosylation, acetylation and phosphorylation. The term “peptide” refers to shorter stretches of amino acids, generally less than about 30 amino acids.

“Regulatory elements” as used herein, refer to nucleotide sequences present in cis, necessary for transcription and/or translation of a nucleotide sequence. In some embodiments, the transcriptional regulatory elements comprise a promoter 5′ of the gene sequence to be expressed, transcriptional initiation and termination sites, and polyadenylation signal sequence. The term “transcriptional initiation site” refers to the nucleic acid in the construct corresponding to the first nucleic acid incorporated into the primary transcript, i.e., the mRNA precursor; the transcriptional initiation site may overlap with the promoter sequences. The term “transcriptional termination site” refers to a nucleotide sequence normally represented at the 3′ end of a gene of interest or the stretch of sequences to be transcribed, that causes RNA polymerase to terminate transcription. The polyadenylation signal sequence, or poly-A addition signal provides the signal for the cleavage at a specific site at the 3′ end of eukaryotic mRNA and the post-transcriptional addition in the nucleus of a sequence of about 100-200 adenine nucleotides (polyA tail) to the cleaved 3′ end. The polyadenylation signal sequence includes the sequence AATAAA located at about 10-30 nucleotides upstream from the site of cleavage, plus a downstream sequence.

The promoter can be constitutive or inducible. An enhancer (i.e., a cis-acting DNA element that acts on a promoter to increase transcription) may be necessary to function in conjunction with the promoter to increase the level of expression obtained with a promoter alone, and may be included as a transcriptional regulatory element. Often, the polynucleotide segment containing the promoter will include the enhancer sequences as well (e.g., CMV IE P/E; SV40 P/E; MPSV P/E). Splice signals may be included where necessary to obtain spliced transcripts. To produce a secreted polypeptide, the selected sequence will generally include a signal sequence encoding a leader peptide that directs the newly synthesized polypeptide to and through the ER membrane where the polypeptide can be routed for secretion. The leader peptide is often but not universally at the amino terminus of a secreted protein and is cleaved off by signal peptidases after the protein crosses the ER membrane. The selected sequence will generally, but not necessarily, include its own signal sequence. Where the native signal sequence is absent, a heterologous signal sequence can be fused to the selected sequence. Numerous signal sequences are known in the art and available from sequence databases such as GenBank and EMBL. Translational regulatory elements include a translational initiation site (AUG), stop codon and poly A signal for each individual polypeptide to be expressed. An internal ribosome entry site (IRES) is included in some constructs. IRES is defined below.

A “transcription unit” defines a region within a construct that contains one or more genes to be transcribed, wherein the genes contained within that segment are operably linked to each other and transcribed from a single promoter, and as a result, the different genes are at least transcriptionally linked. More than one coding region (e.g., coding region of a protein or product, or coding region for a double-stranded RNA complex) can be transcribed from each transcription unit. Each transcription unit will comprise the regulatory elements necessary for the transcription (and translation, if relevant) of any of the coding sequences that are contained within the unit, as well as any additional selectable marker genes that may be operably linked to one or more of the components in the same transcription unit.

A “promoter” refers to a polynucleotide sequence that controls transcription of a gene or sequence to which it is operably linked. A promoter includes signals for RNA polymerase binding and transcription initiation. The promoters used will be functional in the cell type of the host cell in which expression of the selected sequence is contemplated. A large number of promoters including constitutive, inducible and repressible promoters from a variety of different sources, are well known in the art (and identified in databases such as GenBank) and are available as or within cloned polynucleotides (from, e.g., depositiories such as ATCC as well as other commercial or individual sources). With inducible promoters, the activity of the promoter increases or decreases in response to a signal. Among the promoters that have been identified as strong promoters for high-level expression are the SV40 early promoter, adenovirus major late promoter, mouse metallothionein-I promoter, Rous sarcoma virus long terminal repeat, and human cytomegalovirus immediate early promoter (CMV).

An “enhancer”, as used herein, refers to a polynucleotide sequence that enhances transcription of a gene or coding sequence to which it is operably linked. Unlike promoters, enhancers are relatively orientation and position independent and have been found 5′ (Lainins et al., Proc. Natl. Acad. Sci. USA, 78:993 [1981]) or 3′ (Lusky et al., Mol. Cell. Bio., 3:1108 [1983]) to the transcription unit, within an intron (Banerji et al., Cell, 33:729 [1983]) as well as within the coding sequence itself (Osborne et al., Mol. Cell. Bio., 4:1293 [1984]). Therefore, enhancers may be placed upstream or downstream from the transcription initiation site or at considerable distances from the promoter, although in practice enhancers may overlap physically and functionally with promoters. A large number of enhancers, from a variety of different sources are well known in the art (and identified in databases such as GenBank) and available as or within cloned polynucleotide sequences (from, e.g., depositories such as the ATCC as well as other commercial or individual sources). A number of polynucleotides comprising promoter sequences (such as the commonly-used CMV promoter) also comprise enhancer sequences. For example, all of the strong promoters listed above also contain strong enhancers. Bendig, Genetic Engineering, 7:91 (Academic Press, 1988).

The term “intron” as used herein, refers to a non-coding nucleotide sequence of varying length, normally present within many eukaryotic genes, which is removed from a newly transcribed mRNA precursor by the process of splicing. In general, the process of splicing requires that the 5′ and 3′ ends of the intron be correctly cleaved and the resulting ends of the mRNA be accurately joined, such that a mature mRNA having the proper reading frame for protein synthesis is produced. An intron useful in the constructs of this invention will generally be an efficient intron characterized by a splicing efficiency which results in most of the transcripts diverted to expression of the desired product while also providing enough unspliced transcripts for expression of the selectable marker gene (selectable marker gene cloned within and bounded by the ends of, the intron) in amounts sufficient for selection. The efficient intron preferably has a splicing efficiency of about 80 to 99%, preferably about 90-99%. Intron splicing efficiency is readily determined by quantifying the spliced transcripts versus the full-length, unspliced transcripts that contain the intron, using methods known in the art such as by quantitative PCR or Northern blot analysis, using appropriate probes for the transcripts. See, e.g., Sambrook et al., supra, and other general cloning manuals. Reverse transcription-polymerase chain reaction (RT-PCR) can be used to analyze RNA samples containing mixtures of spliced and unspliced mRNA transcripts. For example, fluorescent-tagged primers designed to span the intron are used to amplify both spliced and unspliced targets. The resultant amplification products are then separated by gel electrophoresis and quantitated by measuring the fluorescent emission of the appropriate band(s). A comparison is made to determine the amount of spliced and unspliced transcripts present in the RNA sample.

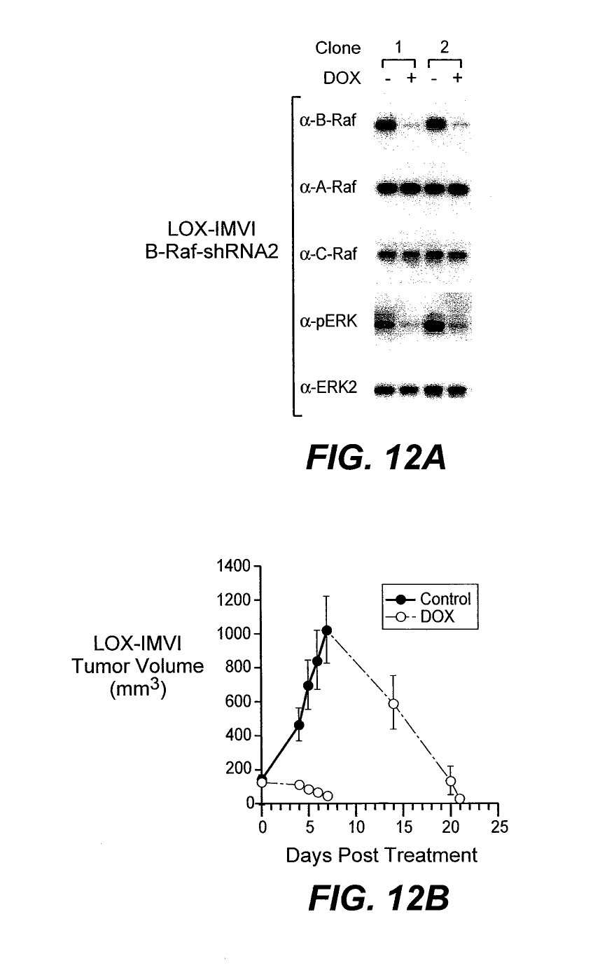

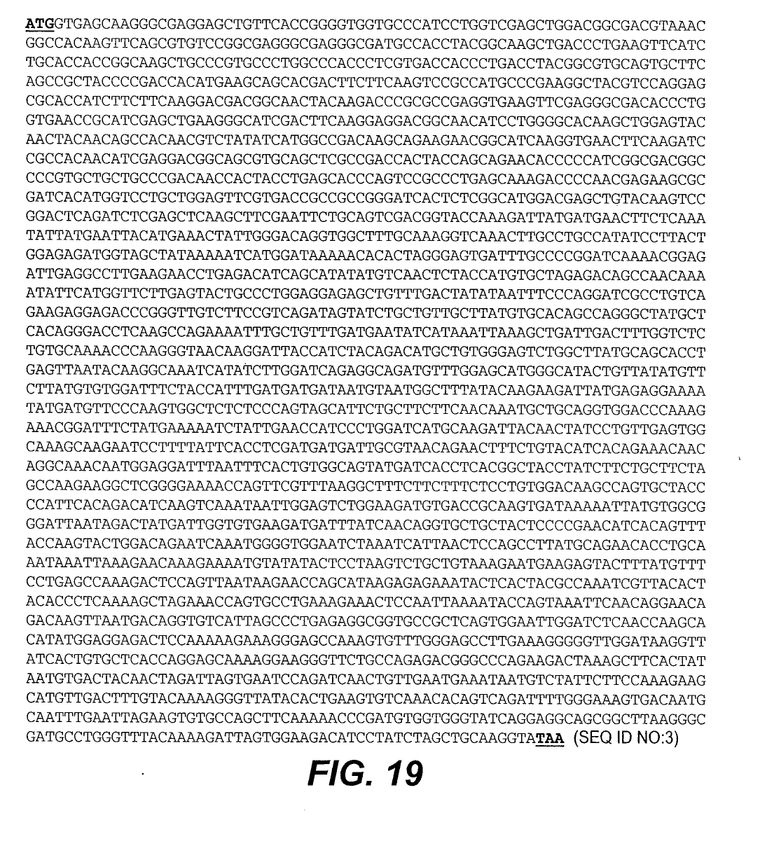

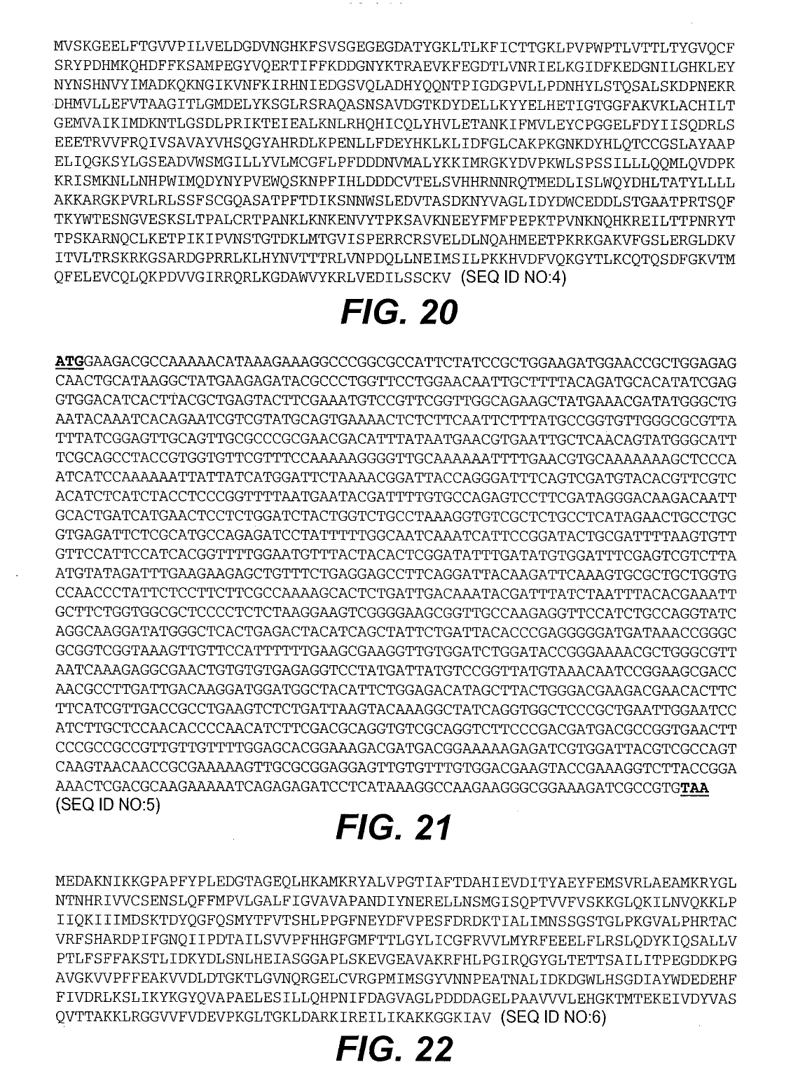

Introns have highly conserved sequences at or near each end of the intron which are required for splicing and intron removal. As used herein “splice donor site” or “SD” or “5′splice site” refers to the conserved sequence immediately surrounding the exon-intron boundary at the 5′ end of the intron, where the exon comprises the nucleic acid 5′ to the intron. The term “splice acceptor site” or “SA” or “3′splice site” herein refers to the sequence immediately surrounding the intron-exon boundary at the 3′ end of the intron, where the exon comprises the nucleic acid 3′ to the intron. An “efficient intron” will comprise a splice donor site and a splice acceptor site that result in splicing of messenger RNA precursors at a frequency between about 80 to 99%, preferably 90 to 95%, more preferably at least 95%, as determined by methods known in the art such as by quantitative PCR. Many splice donor and splice acceptor sites have been characterized and Ohshima et al., J. Mol. Biol., 195:247-259 (1987) provides a review of these. Examples of efficient splice donor sequences include the wild type (WT) ras splice donor sequence and the GAC:GTAAGT sequence. One preferred splice donor site is a “consensus splice donor sequence” and a preferred splice acceptor site is a “consensus splice acceptor sequence”; these consensus sequences are evolutionarily highly conserved. The consensus sequences for both splice donor and splice acceptor sites in the mRNAs of higher eukaryotes are shown in Molecular Biology of the Cell, 3rd edition. Alberts et al. (eds.), Garland Publishing, Inc., New York, 1994, on page 373, FIG. 12-53. The consensus sequence for the 5′ splice donor site is C/A (C or A) AG:GUAAGU (wherein the colon denotes the site of cleavage and ligation). The 3′ splice acceptor site occurs within the consensus sequence (U/C)11NCAG:G. Other efficient splice donor and acceptor sequences can be readily determined using the techniques for measuring the efficiency of splicing.

An “internal ribosome entry site” or “IRES” describes a sequence which functionally promotes translation initiation independent from the gene 5′ of the IRES and allows two cistrons (open reading frames) to be translated from a single transcript in an animal cell. The IRES provides an independent ribosome entry site for translation of the open reading frame immediately downstream (downstream is used interchangeably herein with 3′) of it. Unlike bacterial mRNA which can be polycistronic, i.e., encode several different polypeptides that are translated sequentially from the mRNAs, most mRNAs of animal cells are monocistronic and code for the synthesis of only one protein. With a polycistronic transcript in a eukaryotic cell, translation would initiate from the 5′ most translation initiation site, terminate at the first stop codon, and the transcript would be released from the ribosome, resulting in the translation of only the first encoded polypeptide in the mRNA. In a eukaryotic cell, a polycistronic transcript having an IRES operably linked to the second or subsequent open reading frame in the transcript allows the sequential translation of that downstream open reading frame to produce the two or more polypeptides encoded by the same transcript. The use of IRES elements in vector construction has been previously described, see, e.g., Pelletier et al., Nature 334: 320-325 (1988); Jang et al., J. Virol. 63: 1651-1660 (1989); Davies et al., J. Virol. 66: 1924-1932 (1992); Adam et al. J. Virol. 65: 4985-4990 (1991); Morgan et al. Nucl. Acids Res. 20: 1293-1299 (1992); Sugimoto et al. Biotechnology 12: 694-698 (1994); Ramesh et al. Nucl. Acids Res. 24: 2697-2700 (1996); and Mosser et al. (1997), supra).

“Operably linked” refers to a juxtaposition of two or more components, wherein the components so described are in a relationship permitting them to function in their intended manner. For example, a promoter and/or enhancer is operably linked to a coding sequence if it acts in cis to control or modulate the transcription of the linked sequence. Generally, but not necessarily, the DNA sequences that are “operably linked” are contiguous and, where necessary to join two protein coding regions or in the case of a secretory leader, contiguous and in reading frame. However, although an operably linked promoter is generally located upstream of the coding sequence, it is not necessarily contiguous with it. Enhancers do not have to be contiguous. An enhancer is operably linked to a coding sequence if the enhancer increases transcription of the coding sequence. Operably linked enhancers can be located upstream, within or downstream of coding sequences and at considerable distances from the promoter. A polyadenylation site is operably linked to a coding sequence if it is located at the downstream end of the coding sequence such that transcription proceeds through the coding sequence into the polyadenylation sequence. Linking is accomplished by recombinant methods known in the art, e.g., using PCR methodology, by annealing, or by ligation at convenient restriction sites. If convenient restriction sites do not exist, then synthetic oligonucleotide adaptors or linkers are used in accord with conventional practice.

The term “expression” as used herein refers to transcription or translation occurring within a host cell. The level of expression of a desired product in a host cell may be determined on the basis of either the amount of corresponding mRNA that is present in the cell, or the amount of the desired product encoded by the selected sequence. For example, mRNA transcribed from a selected sequence can be quantitated by PCR or by northern hybridization (see Sambrook et al., Molecular Cloning: A Laboratory Manual, Cold Spring Harbor Laboratory Press (1989)). Protein encoded by a selected sequence can be quantitated by various methods, e.g., by ELISA, by assaying for the biological activity of the protein, or by employing assays that are independent of such activity, such as western blotting or radioimmunoassay, using antibodies that are recognize and bind reacting the protein. See Sambrook et al., 1989, supra.

A “host cell” refers to a cell into which a polynucleotide of the invention is introduced. Host cell includes both prokaryotic cells used for propagation of the construct to prepare plasmid stocks, and eukaryotic cells for expression of the selected sequence. Typically, the eukaryotic cells are mammalian cells.

As used herein the term “codon-optimized coding region” or “codon-optimized” means a nucleic acid coding region that has been adapted for expression in the cells of a given vertebrate by replacing at least one, or more than one, or a significant number, of codons with one or more codons that are more frequently used in the genes of that vertebrate.

TABLE 2

| | | |
|---|---|---|
| TASK | XXXXXXXXXXXXXXX | (Length = 15 amino acids) |
| Comparison | XXXXXYYYYYYY | (Length = 12 amino acids) |
| Protein | | |

% amino acid sequence identity = (the number of identically matching amino acid residues between the two polypeptide sequences as determined by ALIGN-2) divided by (the total number of amino acid residues of the TASK polypeptide) = 5 divided by 15 = 33.3%

TABLE 3

| | | |
|---|---|---|
| TASK | XXXXXXXXXX | (Length = 10 amino acids) |
| Comparison | XXXXXYYYYYYZZYZ | (Length = 15 amino acids) |
| Protein | | |

% amino acid sequence identity = (the number of identically matching amino acid residues between the two polypeptide sequences as determined by ALIGN-2) divided by (the total number of amino acid residues of the TASK polypeptide) = 5 divided by 10 = 50%

TABLE 4

| | | |
|---|---|---|
| TASK-DNA | NNNNNNNNNNNNNN | (Length = 14 nucleotides) |
| Comparison | NNNNNNLLLLLLLLLL | (Length = 16 nucleotides) |
| DNA | | |

% nucleic acid sequence identity = (the number of identically matching nucleotides between the two nucleic acid sequences as determined by ALIGN-2) divided by (the total number of nucleotides of the TASK-DNA nucleic acid sequence) = 6 divided by 14 = 42.9%

TABLE 5

| | | |
|---|---|---|
| TASK-DNA | NNNNNNNNNNNN | (Length = 12 nucleotides) |
| Comparison DNA | NNNNLLLVV | (Length = 9 nucleotides) |

% nucleic acid sequence identity = (the number of identically matching nucleotides between the two nucleic acid sequences as determined by ALIGN-2) divided by (the total number of nucleotides of the TASK-DNA nucleic acid sequence) = 4 divided by 12 = 33.3%

### II. Compositions and Methods of the Invention

In one aspect, this invention generally relates to vectors that permit the regulated, inducible expression of a RNA coding sequence (encoding, for example, a shRNA or miRNA), or a polypeptide, and methods using these vectors. The vectors generally include a polynucleotide encoding the Tet repressor (TetR) protein and a promoter comprising one or more Tet operon (TetO) sequences.

In another aspect, the invention generally relates to a codon-optimized TetR coding region, and vectors comprising this codon-optimized TetR coding region.

In another aspect, the invention generally relates to a modified H1 promoter comprising two tet operon sites, and vectors comprising this modified H1 promoter.

The invention further relates generally to methods to express within a cell a RNA molecule(s) or polypeptide.

Many variations of the basic construct design are possible and examples will be described in detail below. One of skill in the art will recognize that modifications of the present vectors can be made without departing from the scope of the invention. It will also be understood that desirable features that facilitate cloning can be genetically engineered into the genes of interest and the vectors by methods routine in the art of recombinant DNA methodology.

The RNA coding region preferably encodes a double-stranded RNA complex (e.g., siRNA, miRNA, shRNA) that is capable of down-regulating the expression of a particular gene or genes. In some embodiments, a double-stranded RNA complex is expressed in the form of an RNA molecule having a stem-loop or a so-called “hairpin” structure. As used herein, “hairpin” structure encompasses shRNAs and miRNAs. In some embodiments, a double-stranded RNA complex is expressed in the form of separate complementary or partially complementary RNA strands.

Methods are well-known in the art for designing double-stranded RNA complexes, e.g., siRNA, miRNA, and shRNAs. For example, resources and citations describing the design of effective shRNA and siRNA are found in Sandy et al, BioTechniques 39:215-224 (2005). It is understood that the sequences of a double-stranded RNA complex may be of natural origin or may be synthetic. For example, Example 13 discloses a hybrid miRNA comprising a synthetic double stranded portion embedded in the backbone of a naturally occurring microRNA.

The RNA complex comprises a double-stranded region corresponding to a region of a gene to be down-regulated is expressed in the cell. One strand of the RNA double-stranded region is substantially identical (typically at least about 80%, 85%, 90%, 91%, 92%, 93%, 94%, 95%, 96%, 97%, 98%, 99% identical) in sequence to the sequence of the coding region targeted for down regulation. The other strand of the double-stranded region (interchangeably termed “RNA double-stranded region) is complementary to the sequence of the coding region targeted for down regulation, or partially complementary to the coding region targeted for down regulation (typically at least about 80%, 85%, 90%, 91%, 92%, 93%, 94%, 95%, 96%, 97%, 98%, 99% identical to the complement of the coding region targeted). It is understood that the double-stranded region can be formed by two separate RNA stranded, or by the self-complementary portions of a single RNA having a hairpin structure. The double-stranded region is generally at least about 15 nucleotides in length and, in some embodiments, is about 15 to about 30 nucleotides in length. However, a significantly longer double-stranded region can be used effectively in some organisms. In a more preferred embodiment, the double-stranded region is between about 19 and 22 nucleotides in length. The double-stranded region is preferably identical to the target nucleotide sequence over this region.

When the coding region to be down regulated is in a family of highly conserved genes, the sequence of the RNA double-stranded region can be chosen with the aid of sequence comparison to target only the desired gene. On the other hand, if there is sufficient identity among a family of homologous genes within an organism, a double-stranded can be designed that would down regulate a plurality of genes simultaneously.

In some embodiments, a single RNA coding region in the construct serves as a template for the expression of a self-complementary hairpin RNA, comprising a sense region, a loop region and an antisense region. The sense and antisense regions are each preferably about 15 to about 30 nucleotides in length. The loop region preferably is about 2 to about 15 nucleotides in length, more preferably from about 4 to about 9 nucleotides in length. Following expression the sense and antisense regions form a duplex.

In another embodiment, the vector comprises two RNA coding regions. The first coding region is a template for the expression of a first RNA and the second coding region is a template for the expression of a second RNA. Following expression, the first and second RNAs form a duplex. The retroviral construct preferably also comprises a first Pol III promoter operably linked to the first RNA coding region and a second Pol III promoter operably linked to the second RNA coding region.

It is understood that, in certain embodiments, a vector of the invention can encompass nucleic acid sequences sufficient to form more than RNA coding region that inhibit expression of distinct target genes. In this embodiment, simultaneous inhibition of distinct target genes can be accomplished with a single vector of the invention. The number of different RNA complex transcripts that can be expressed simultaneously is limited only by the packaging capacity of the vector (if a viral vector is used) and adjacent promoters, including any of the promoters described below, can be selected to eliminate or minimize interference and allow for efficient simultaneous inhibition of multiple target genes. The inhibition of multiple RNA construct transcripts of adjacent promoters, for example, 2 or more, 3 or more, 4 or more, 5 or more, 6 or more, 7 or more, 8 or more, 9 or more, or or more adjacent promoters allows the user to generate a desire phenotype that develops only when several coding regions (e.g., genes) are targeted simultaneously and enables manipulation and elucidation of complex genetic systems.

In addition, it is understood that a single cell can be generated that comprises more than one vector of the invention.

In yet another embodiment of the invention, the vector comprises a first RNA Pol III promoter operably linked to a first RNA coding region, and a second RNA Pol III promoter operably linked to the same first RNA coding region in the opposite direction, such that expression of the RNA coding region from the first RNA Pol III promoter results in a synthesis of a first RNA molecule as the sense strand and expression of the RNA coding region from the second RNA Pol III promoter results in synthesis of a second RNA molecule as an antisense strand that is, substantially complementary to the first RNA molecule. In one such embodiment, both RNA Polymerase III promoters are separated from the RNA coding region by termination sequences, preferably termination sequences having five consecutive T residues.

In further embodiments an RNA double-stranded region is expressed using two or more vectors. In one embodiment, a first retroviral construct is used that directs the expression of a first RNA and a second retroviral construct is used that directs expression of a second RNA that is complementary to the first. Following expression the first and second RNAs form a double-stranded region.

The RNA double-stranded region may be flanked by single stranded regions on one or both sides of the double-stranded region. For example, in the case of the hairpin, the single stranded loop region would connect the double-stranded region at one end.

The RNA coding region is generally operatively linked to a terminator sequence. The pol III terminators preferably comprise of stretches of 4 or more thymidine (“T”) residues. In a preferred embodiment, a cluster of 5 consecutive Ts is linked immediately downstream of the RNA coding region to serve as the terminator. In such a construct pol III transcription is terminated at the second or third T of the DNA template, and thus only 2 to 3 uridine (“U”) residues are added to the 3′ end of the coding sequence.

The sequence of the RNA coding region, and thus the sequence of the RNA double-stranded region, may be chosen to be complementary or partially complementary to the coding region whose expression is to be downregulated in a cell or organism. The degree of down regulation achieved with a given RNA duplex sequence for a given target gene will vary by sequence. One of skill in the art will be able to readily identify an effective sequence, e.g., by using methods exemplified or described herein.

A promoter useful in the present invention can comprise a promoter of eukaryotic or prokaryotic origin that can provide high levels of constitutive expression across a variety of cell types and will be sufficient to direct the transcription of a distally located sequence, which is a sequence linked to the 5, end of the promoter sequence in a cell. The promoter region can also include control elements for the enhancement or repression of transcription and can be modified as desired by the user and depending on the context. Suitable promoters include, for example, RNA polymerase (pot) III promoters including, but not limited to, the human and murine U6 pot III promoters as well as the human and marine H1 RNA pot III promoters; RNA polymerase (pol) II promoters, cytomegalovirus immediate early promoter (pCMV), the Rous Sarcoma virus long terminal repeat promoter (pRSV), and the SP6, T3, and T7 promoters. In addition, a hybrid promoter also can be prepared that contains elements derived from, for example, both a RNA I polymerase (pot) III promoter and an RNA polymerase (pot) II promoter. Description of additional RNA Polymerase III promoters can be found, for example, in Paule and White. Nucleic Acids Research., Vol 28, pp 1283-1298 (2000), which is hereby incorporated by reference in its entirety.

In some embodiments, the promoter is a modified H1 promoter comprising a Tet operon sequence, e.g., promoter H1-tetO₂-1x or promoter H1-tetO₂-2x described herein. In some embodiments, the promoter comprises the polynucleotide shown in SEQ ID NO:21.

Modified promoters that contain sequence elements derived from two or more naturally occurring promoter sequences can be combined by the skilled person to effect transcription under a desired set of conditions or in a specific context.

Enhancer sequences upstream from the promoter or terminator sequences downstream of the coding region can be optionally be included in the; vectors of the present invention to facilitate expression. Vectors of the present invention can also contain additional nucleic acid sequences, such as a polyadenylation sequence, a localization sequence, or a signal sequence, sufficient to permit a cell to efficiently and effectively process the protein expressed by the nucleic acid of the vector. Such additional sequences can be inserted into the vector such that they are operably linked with the promoter sequence, if transcription is desired, or additionally with the initiation and processing sequence if translation and processing are desired. Alternatively, the inserted sequences can be placed at any position in the vector.

As used herein, an “inducible promoter” refers to a transcriptional control element that can be regulated in response to specific signals. An inducible promoter is transcriptionally active when bound to a transcriptional activator, which in turn is activated under a specific set of conditions, for example, in the presence of a particular combination of chemical signals that affect binding of the transcriptional activator to the inducible promoter and/or affect function of the transcriptional activator itself. Thus, an inducible promoter is a promoter that, either in the absence of an inducer, does not direct expression, or directs low levels of expression, of a nucleic acid sequence to which the inducible promoter is operably linked; or exhibits a low level of expression in the presence of a regulating factor that, when removed, allows high-level expression from the promoter, for example, the system. In the presence of an inducer, an inducible promoter directs transcription at an increased level. Inducible promoters are described herein, including supra.

A TetR/TetO elements may be used to confer regulatable expression. In the ‘off’ state, the Tet repressor protein (TetR) binds the modified polIII promoter, thereby preventing siRNA expression. However, in the presence of tetracycline or a tetracycline analog, doxycycline (Dox), the Tet repressor protein is released from the promoter, permitting transcription.

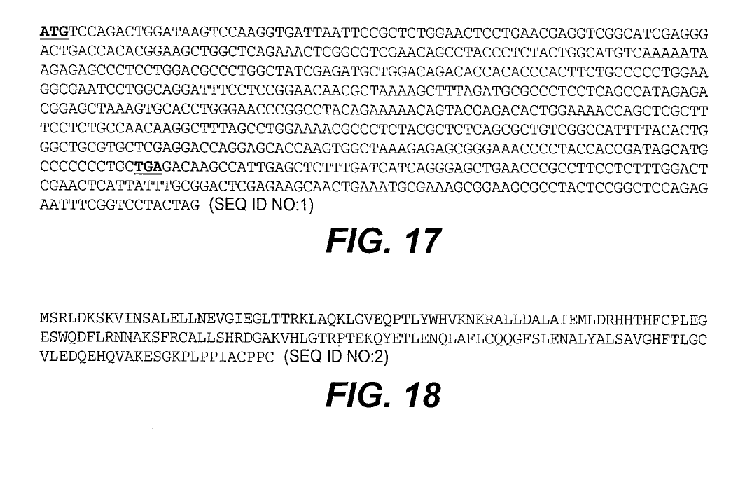

In some embodiments, the vector may comprise a nucleic acid sequence encoding the Tet repressor, wherein the polynucleotide sequence has been codon optimized for mammalian expression. Vectors comprising a codon optimized Tet repressor sequence are described and exemplified herein. A sequence for the mammalian codon-optimized TetR is shown in FIG. 17 (SEQ ID NO:1). Deviations in the nucleotide sequence that comprise the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). The “genetic code” which shows which codons encode which amino acids is reproduced herein as Table 2. As a result, many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the proteins encoded by the DNA.

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

Given the large number of gene sequences available for a wide variety of animal, plant and microbial species, it is possible to calculate the relative frequencies of codon usage. Codon usage tables are readily available. See Nakamura, Y., et al. “Codon usage tabulated from the international DNA sequence databases: status for the year 2000 “Nucl. Acids Res. 28:292 (2000).

By utilizing these or similar tables, one of ordinary skill in the art can apply the frequencies to any given polypeptide sequence, and produce a nucleic acid fragment of a codon-optimized coding region which encodes the polypeptide, but which uses codons more optimal for a given species. Codon-optimized coding regions can be designed by various different methods known in the art, some of which are described herein and in U.S. Patent Application publication No. 20040209241.

The sequence of the wildtype Tet repressor protein is known in the art, and is deposited at GenBank under Accession No. J01830. Assays for testing TetR protein binding to the Tet operon sequences are described in Lederer et al, Anal. Biochemistry 232:190-196 (1995).

The function of a promoter can be further modified, if desired, to include appropriate regulatory elements to provide for the desired level of expression or replication in the host cell. For example, appropriate promoter and enhancer elements can be chosen to provide for constitutive, inducible or cell type-specific expression. Useful constitutive promoter and enhancer elements for expression of a target gene transcript include, for example, RSV, CMV, CAG, SV40 and IgH elements. Other constitutive, inducible and cell type-specific regulatory elements are well known in the art.

A promoter that is particularly useful in the vector of the invention is compatible with mammalian genes and, further, can be compatible with expression of genes from a wide variety of species. For example, a promoter useful for practicing the invention can be a promoter of the eukaryotic RNA polymerases pol II and pol III, or a hybrid thereof.

The RNA polymerase III promoters have a transcription machinery that is compatible with a wide variety of species, a high basal transcription rate and recognize termination sites with a high level of accuracy. For example, the human and murine U6 RNA polymerase (pot) III and H1 RNA pot III promoters are well characterized and useful for practicing the invention. As exemplified below, because the activities of these two promoters as well as the localization of expressed nucleic acid sequences can vary from cell type to cell type, if desired, U6 and H1 Antiviral vectors of the invention can be prepared and targeted to the desired cells for target gene inhibition. One skilled in the art will be able to select and/or modify the promoter that is most effective for the desired application and cell type so as to optimize target gene inhibition. Thus, promoters that are useful in the invention include those promoters that are sufficient to render promoter-dependent gene expression controllable for cell-type specificity, cell-stage specificity, or tissue-specificity, and those promoters that are inducible by external signals or agents, for example, metallothionein, MMTV, and pENK promoters. The promoter sequence can be one that does not occur in nature, so long as it functions in a mammalian cell.

In particular embodiments, intracellular transcription of double-stranded RNA complexes can be achieved by cloning the double-stranded RNA complex coding sequences into RNA pot III transcription units, which normally encode the smaller nucleic RNA (snRNA) U6 or the human RNAse P RNA H1. The U6 and H1 promoters are members of the type III class of Pol III promoters. The U6 and H1 are different in size but contain the same conserved sequence elements or protein binding sites. The +1 nucleotide of the U6-like promoters is always guanosine, whereas the +1 for H1 promoters is adenosine. The termination signal for these promoters is defined by 5 thymidines, and the transcript is typically cleaved after the second uridine. Cleavage at this position generates a 3′ overhang in the expressed double-stranded RNA complex, which is similar to the 3′overhangs of synthetic siRNAs. Any sequence of up to about 400 nucleotides in length can be transcribed by the polIII promoters, therefore they are ideally suited for the expression of the nucleic acid sequences that are subject of the invention.

In other embodiments, e.g., embodiments involving microRNA expression or protein expression, intracellular transcription of double-stranded RNA complexes can be achieved by cloning the double-stranded RNA complex templates into RNA pol II transcription units. Suitable pol II promoters are well known in the art, and include, e.g., viral promoters obtained from the genomes of viruses include promoters from polyoma virus, fowlpox virus (UK 2,211,504 published 5 Jul. 1989), adenovirus (such as Adenovirus 2 or 5), herpes simplex virus (thymidine kinase promoter), bovine papilloma virus, avian sarcoma virus, cytomegalovirus, a retrovirus (e.g., MoMLV, or RSV LTR), Hepatitis-B virus, Myeloproliferative sarcoma virus promoter (MPSV), VISNA, and Simian Virus 40 (SV40). Other heterologous mammalian promoters include, e.g., the actin promoter, immunoglobulin promoter, heat-shock protein promoters. The aforementioned promoters are known in the art. The early and late promoters of the SV40 virus are conveniently obtained as a restriction fragment that also contains the SV40 viral origin of replication. Fiers et al., Nature, 273:113 (1978); Mulligan and Berg, Science, 209:1422-1427 (1980); Pavlakis et al., Proc. Natl. Acad. Sci. USA, 78:7398-7402 (1981). The immediate early promoter of the human cytomegalovirus (CMV) is conveniently obtained as a HindIII E restriction fragment. Greenaway et al., Gene, 18:355-360 (1982). A broad host range promoter, such as the SV40 early promoter or the Rous sarcoma virus LTR, is suitable for use in the present expression vectors. Generally, a strong promoter is employed to provide for high level transcription and expression of the desired product. Among the eukaryotic promoters that have been identified as strong promoters for high-level expression are the SV40 early promoter, adenovirus major late promoter, mouse metallothionein-I promoter, Rous sarcoma virus long terminal repeat, and human cytomegalovirus immediate early promoter (CMV or CMV IE). In a preferred embodiment, the promoter is a SV40 or a CMV early promoter.

The promoter that drives expression of the double-stranded RNA complex transcript in the target cell can further be useful to restrict expression to a specific time, cell type or tissue. If desired, regulatable transcriptional elements can be incorporated into a vector of the invention that can be switched on and off via exogenous stimuli. The regulatable systems can be based on naturally occurring inducible promoters that exhibit tissue specificity or consist of chimeric systems, which contain pro- and eukaryotic elements from different organisms as described in Aga-Mohamad and Lotte, J. Cain. Invest. 105:1177-83 (2000), which is incorporated herein by reference.

The transcriptional regulatory region in higher eukaryotes may comprise an enhancer sequence. Many enhancer sequences from mammalian genes are known e.g., from globin, elastase, albumin, α-fetoprotein and insulin genes. A suitable enhancer is an enhancer from a eukaryotic cell virus. Examples include the SV40 enhancer on the late side of the replication origin (bp 100-270), the enhancer of the cytomegalovirus immediate early promoter (Boshart et al. Cell 41:521 (1985)), the polyoma enhancer on the late side of the replication origin, and adenovirus enhancers. See also Yaniv, Nature, 297:17-18 (1982) on enhancing elements for activation of eukaryotic promoters. The enhancer sequences may be introduced into the vector at a position 5′ or 3′ to the gene of interest, but is preferably located at a site 5′ to the promoter.

It is further contemplated that a vector of the invention can be prepared by incorporating a recombination system, for example, the Cre/lox system of bacteriophage Pi, the FLP/FRT system of the yeast 2 uM plasmid, the 1/RS system of the yeast plasmid pSR1, or the modified Gin/gix system of bacteriophage Mu. In a particular embodiment exemplified herein, an inducible lentiviral vector of the invention is prepared that incorporates the Cre/loxP recombination system. Briefly, Cre is a 38 kDa recombinase protein from bacteriophage Pi which mediates intramolecular (excisive or inversional) and intermolecular (integrative) site specific recombination between loxP sites as described by Sauer, Methods of Enzymology, 225: 890-900 (1993), which is incorporated herein by reference. A loxP site (locus of X-ing over) consists of two 13 bp inverted repeats separated by an 8 bp asymmetric spacer region. One molecule of Cre binds per inverted repeat or two Cre molecules line up at one loxP site. The recombination occurs in the 8 base pair asymmetric spacer region, which also is responsible for the directionality of the site. Two loxP sequences in opposite orientation to each other invert the intervening piece of DNA, two sites in direct orientation dictate excision of the intervening DNA between the sites leaving one loxP site behind.

The ability to excise a piece of nucleic acid sequence at a particular time can be exploited by flanking a nucleic acid sequence with a pair of lox P sites and introduce the enzyme when excision is desired. If desired, a Cre transgene can be put under control of an inducible and/or tissue specific promoter to allow excision of a nucleic acid sequence in selected cells and at selected times. As described herein, an inducible lentiviral vector of the invention can include a nucleic acid sequence that serves as a stuffer fragment between the promoter and the double-stranded RNA complex hairpin. The stuffer fragment can be flanked by loxP sites, so that a CRE mediated recombination event leads to excision of the stuffer nucleic acid sequence and juxtaposition of the RNA coding region and the promoter, resulting in target gene inhibition. A vector of the invention can be used for any application, for example, somatic gene therapy where the transient controllable expression of a vector of the invention is desirable. In addition, a vector of the invention can be used to dissect complex biological problems in vivo, e.g., if it allows for inhibition target genes in a tissue specific manner by putting CRE under the control of a tissue specific promoter. In this embodiment of the invention, a vector of the invention can be utilized for focused target gene inhibition in specific regions of a tissue.

Thus, a vector of the invention can further encompass nucleic acid sequences sufficient for induction by a site-specific recombinase, for example, ore recombinase. In this embodiment induction of the promoter that drives double-stranded RNA complex expression is initiated by contacting the lentiviral vector with a recombinase that mediates a recombination event that involves excision of a stuffer nucleic acid sequence and results in juxtaposition of the promoter and the corresponding first and second nucleic acid sequences driven by the promoter so as to allow transcription and formation of a double-stranded RNA coding region capable of inhibiting the expression of a target gene.

Introns suitable for use in the present invention are suitably prepared by any of several methods that are well known in the art, such as isolation from a naturally occurring nucleic acid or de novo synthesis. The introns present in many naturally occurring eukaryotic genes have been identified and characterized. Mount, Nucl. Acids Res., 10:459 (1982). Artificial introns comprising functional splice sites also have been described. Winey et al., Mol. Cell. Biol., 9:329 (1989); Gatermann et al., Mol. Cell. Biol., 9:1526 (1989). Introns may be obtained from naturally occurring nucleic acids, for example, by digestion of a naturally occurring nucleic acid with a suitable restriction endonuclease, or by PCR cloning using primers complementary to sequences at the 5′ and 3′ ends of the intron. Alternatively, introns of defined sequence and length may be prepared by in vitro deletion mutagenesis of an existing intron, or synthetically using various methods in organic chemistry. Narang et al, Meth. Enzymol., 68:90 (1979); Caruthers et al, Meth. Enzymol., 154:287 (1985); Froehler et al, Nucl. Acids Res., 14:5399 (1986).

In one embodiment, the intron used is the intron of the vector pRK which contains a SD derived from the CMV immediate early gene and a SA site from an IgG H chain variable region gene, as described in Lucas et al., Nucl. Acids Res. 24: 1774-1779 (1996), Suva et al., Science 237: 893-896 (1997), and U.S. Pat. No. 5,561,053. The selectable gene or fusion gene is inserted within the intron using any of the various known methods for modifying a nucleic acid in vitro. Genes can be inserted into the intron outside of the consensus sequence and without interrupting the sequences important for splicing. Typically, a selectable gene will be introduced into an intron by first cleaving the intron with a restriction endonuclease, and then covalently joining the resulting restriction fragments to the selectable gene in the correct orientation for host cell expression, for example by ligation with ligase. If convenient restriction sites are lacking within the intron, they can be introduced using linkers and oligonucleotides by PCR, ligation or restriction and annealing. An example of intron modification is described in Lucas et al., 1996, supra.

The IRES can be of varying length and from various sources, e.g., encephalomyocarditis virus (EMCV) or picornavirus genomes. Various IRES sequences and their construction are described in, e.g., Pelletier et al., Nature 334: 320-325 (1988); Jang et al., J. Virol. 63: 1651-1660 (1989); Davies et al., J. Virol. 66: 1924-1932 (1992); Adam et al. J. Virol. 65: 4985-4990 (1991); Morgan et al. Nucl. Acids Res. 20: 1293-1299 (1992); Sugimoto et al. Biotechnology 12: 694-698 (1994); and Ramesh et al Nucl. Acids Res. 24: 2697-2700 (1996); and Mosser et al. (1997), supra). In one embodiment, the IRES of ECMV is used in the vectors of the invention. The downstream coding sequence will be operably linked to the IRES, for example, at about 8 bases or more downstream of the 3′ end of the IRES or at any distance that will not negatively affect the expression of the downstream gene. The optimum or permissible distance between the IRES and the start of the downstream gene can be readily determined by varying the distance and measuring expression as a function of the distance.

As used herein in reference to a vector of the, invention, the terms “stuffer nucleic acid sequence” and “stuffer fragment” refer to a nucleic acid sequence that is inserted into or proximal to a promoter sequence driving expression of a RNA coding region and that further contains a transcription stop signal specific to the promoter. The presence of the stuffer fragment thus prevents transcription of the RNA coding region from its corresponding promoter and keeps the promoter-RNA coding region transcription unit in an inactive state. Conversely, upon addition of a recombinase enzyme site specific excision of the stuffer fragment containing the promoter specific transcription stop signal results in juxtaposition of the promoter and its corresponding RNA coding region, resulting in transcription of the RNA coding region and expression of the RNA duplex capable of target gene inhibition.

A stuffer fragment can be any nucleic acid sequence and preferably is a relatively inert sequence that is not prone to conformational changes. For example, a stuffer sequence can be a segment of the lacZ gene or any other: desired nucleic acid segment provided the transcription stop signal that is specific to the promoter driving the double-stranded RNA complex nucleic acid transcription is encompassed and functional in preventing transcription. If desired by the user, the stuffer fragment can contain additional features, for example, a selectable marker that allows for easy detection and determination of the transcriptional state as induced versus non-induced.

The size of a stuffer fragment can be 500 base pairs or more, 600 base pairs or more, 700 base pairs or more, 800 base pairs or more, 1000 base pairs or more, 1200 base pairs or more, 1400 base pairs or more, as long as there is no impairment of its ability to prevent transcription through presence of the promoter specific transcription stop signal or being excised in an enzyme; mediated recombination event. An example of a stuffer fragment is a 1 kb segment of the lacZ gene that contains a sequence consisting of five adjacent Thymines corresponding to a mU6 promoter specific transcription stop signal.

Additional chimeric-regulated systems useful in the invention are known in the art and include, for example, the progesterone system is based on a mutated human progesterone receptor; the insect ecdysone-responsive system; the Bombyx-derived ecdysone-responsive system (BmEcR); and the rapamycin-regulated transcriptional system. These and other regulatable systems: are known in the art and have been described, for example, by Wang et al., Proc. Natl. Acad. Sci. USA 91:8180-84 (1994); No et al., Proc.

The expression vectors described herein will typically contain prokaryotic sequences that facilitate the propagation of the vector in bacteria. Therefore, the vector may have other components such as an origin of replication (i.e., a nucleic acid sequence that enables the vector to replicate in one or more selected host cells) and antibiotic resistance genes for selection in bacteria. Additional eukaryotic selectable gene(s) may be incorporated. Generally, in cloning vectors the origin of replication is one that enables the vector to replicate independently of the host chromosomal DNA, and includes origins of replication or autonomously replicating sequences. Such sequences are well known, e.g., the ColE1 origin of replication in bacteria. Various viral origins (SV40, polyoma, adenovirus, VSV or BPV) are useful for cloning vectors in mammalian cells. Generally, a eukaryotic replicon is not needed for expression in mammalian cells unless extrachromosomal (episomal) replication is intended (the SV40 origin may typically be used only because it contains the early promoter).

The present constructs can accommodate a wide variety of nucleotide sequence inserts. To facilitate insertion and expression of different genes of interest from the constructs and expression vectors of the invention, the constructs are designed with at least one cloning site for insertion of any gene of interest. Preferably, the cloning site is a multiple cloning site, i.e., containing multiple restriction sites. DNA cassettes containing multiple cloning sites can be isolated from commercially available cloning vectors. Gateway sites may also be added, permitting insertion of sequences using a lambda recombination reaction.

Desired sequences of interest may be obtained from phage display libraries, cDNA or genomic DNA libraries. The gene or sequence of interest can be isolated by PCR methods using suitable primers, or they can be chemically synthesized. Libraries can be screened with probes (such as antibodies or oligonucleotides) designed to identify the selectable gene or the product gene (or the protein(s) encoded thereby). Screening the cDNA or genomic library with the selected probe may be conducted using standard procedures as described in chapters 10-12 of Sambrook et al., Molecular Cloning: A Laboratory Manual (New York: Cold Spring Harbor Laboratory Press, 1989).

It is understood that the elements described above are linked in proper reading frame. Further, it is understood that the vectors of the invention can have addition of sequences and sites that facilitate construction and cloning or optimize expression in the selected host cell.

Most expression vectors are capable of replication in at least one class of organism but can be transfected into another organism for expression. For example, a vector is cloned in E. coli and then the same vector is transfected into yeast or mammalian cells for expression even though it is not capable of replicating independently of the host cell chromosome.

For analysis to confirm correct sequences in the constructs, plasmids from the transformants are prepared, analyzed by restriction, and/or sequenced by methods known in the art.

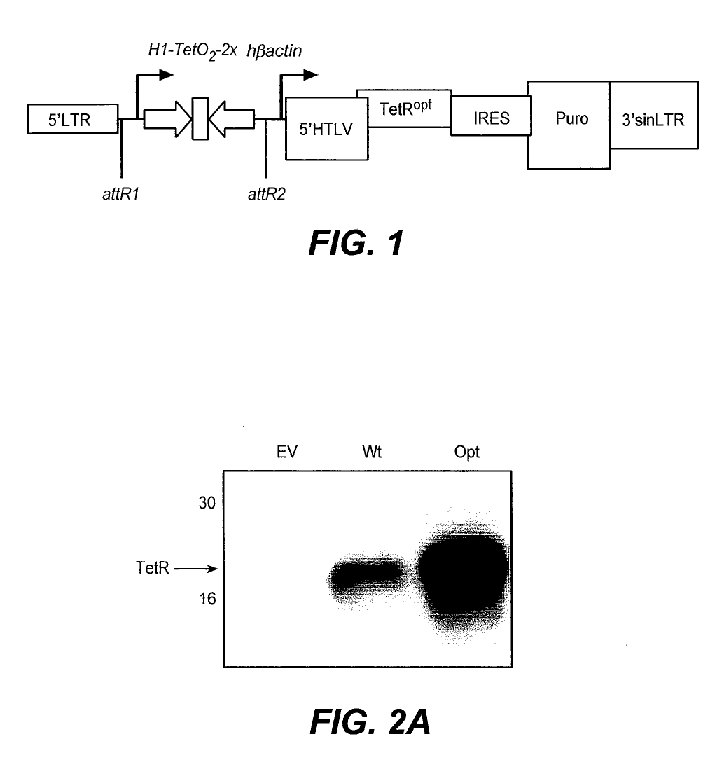

In one aspect of the invention, a recombinant retrovirus is used to deliver a transgene. In some embodiments, the transgene comprises an RNA coding region of interest to a target cell. In some embodiments, the transgene comprises a protein-coding region operably linked to a polIII promoter. In some embodiments, the expression of the transgene is inducible, e.g., the transgene is operably linked to a modified promoter that permits regulation of expression by the TetR protein. In some embodiments, the transgene further comprises a tetR coding region. Retroviral vectors are described and exemplified herein, including in FIGS. 1, 9A, 16A and B, and 10A.

Preferably the target cell is a mammalian cell. The cell may be a primary cell, or may be a cultured cell, for example, an HEK, CHO, COS, MEF, 293 cell. In one embodiment the target cell is an oocyte or an embryonic cell, more preferably a one-cell embryo. The RNA coding region and any associated genetic elements are thus integrated into the genome of the target cell as a provirus. When the target cell is an embryo, the cell may then be allowed to develop into a transgenic animal by methods well known in the art.

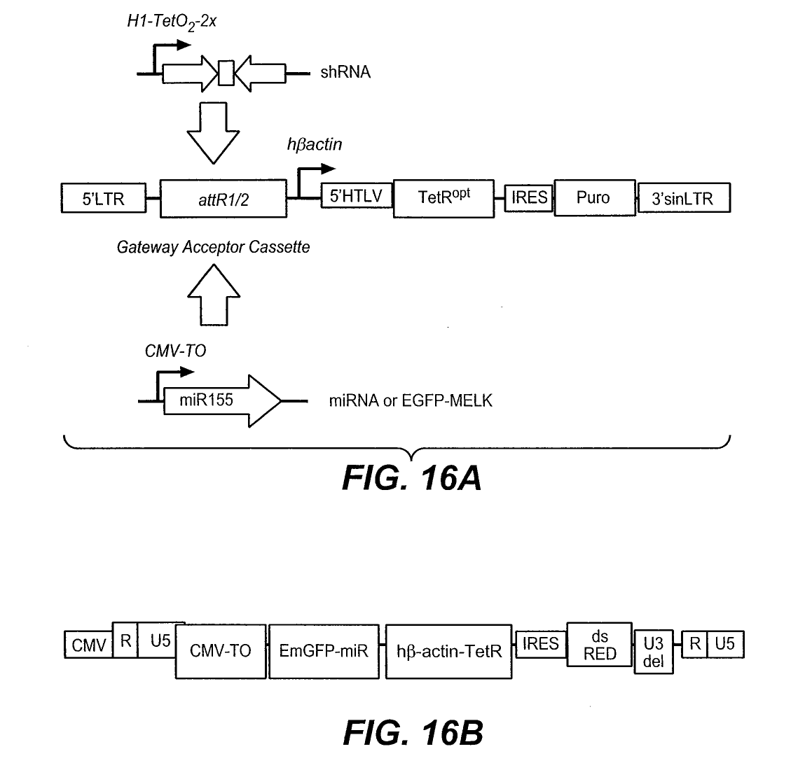

The recombinant retrovirus used, e.g., to deliver a RNA coding region, is preferably a modified lentivirus, and thus is able to infect both dividing and non-dividing cells. Exemplary lentiviral vectors are depicted in FIGS. 16A and B. The recombinant retrovirus preferably comprises a modified lentiviral genome that includes the transgene. Further, the modified lentiviral genome preferably lacks endogenous genes for proteins required for viral replication, thus preventing undesired replication, such as replication in a resulting transgenic animal. The required proteins are preferably provided in trans in the packaging cell line during production of the recombinant retrovirus, as described below.

In another embodiment, the recombinant retrovirus used to deliver the RNA coding region is a modified Moloney virus, for example a Moloney Murine Leukemia Virus. In a further embodiment, the virus is a Murine Stem Cell Virus (Hawley, R. G., et al. (1996) Proc. Natl. Acad. Sci. USA 93:10297-10302; Keller, G., et al. (1998) Blood 92:877-887; Hawley, R. G., et al. (1094) Gene Ther. 1:136-138). The recombinant retrovirus also can be a hybrid virus such as that described in Choi, J K; Hoanga, N; Vilardi, A M; Conrad, P; Emerson, S G; Gewirtz, A M. (2001) Hybrid HIV/MSCV LTR Enhances Transgene Expression of Lentiviral Vectors in Human CD34+ Hematopoietic Cells. Stem Cells 19, No. 3, 236-246.

In the preferred embodiment the transgene is incorporated into a viral construct that comprises an intact retroviral 5′ LTR and a self-inactivating 3′ LTR. The viral construct is preferably introduced into a packaging cell line that packages viral genomic RNA based on the viral construct into viral particles with the desired host specificity. Viral particles are collected and used to infect the host cell. Each of these aspects is described in detail below.

The viral construct is a nucleotide sequence that comprises sequences necessary for the production of recombinant retrovirus in a packaging cell. In one embodiment the viral construct additionally comprises genetic elements that allow for the desired expression of an RNA molecule or gene of interest in the host.

Generation of the viral construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)), Coffin et al. (Retroviruses. Cold Spring Harbor Laboratory Press, N.Y. (1997)) and “RNA Viruses: A Practical Approach” (Alan J. Cann, Ed., Oxford University Press, (2000)).

The viral construct may incorporate sequences from the genome of any known organism. The sequences may be incorporated in their native form or may be modified in any way. For example, the sequences may comprise insertions, deletions or substitutions. In the preferred embodiment the viral construct comprises sequences from a lentivirus genome, such as the HIV genome or the SIV genome.

The viral construct preferably comprises sequences from the 5′ and 3′ LTRs of a lentivirus. More preferably the viral construct comprises the R and U5 sequences from the 5′ LTR of a lentivirus and an inactivated or self-inactivating 3′ LTR from a lentivirus. The LTR sequences may be LTR sequences from any lentivirus from any species. For example, they may be LTR sequences from HIV, SIV, FIV or BIV. Preferably the LTR sequences are HIV LTR sequences. The virus also can incorporate sequences from MMV or MSCV.

The viral construct preferably comprises an inactivated or self-inactivating 3′ LTR. The 3′ LTR may be made self-inactivating by any method known in the art. In the preferred embodiment the U3 element of the 3′ LTR contains a deletion of its enhancer sequence, preferably the TATA box, Sp1 and NF-kappa B sites. As a result of the self-inactivating 3′ LTR, the provirus that is integrated into the host cell genome will comprise an inactivated 5′ LTR.

Optionally, the U3 sequence from the lentiviral 5′ LTR may be replaced with a promoter sequence in the viral construct. This may increase the titer of virus recovered from the packaging cell line. An enhancer sequence may also be included. Any enhancer/promoter combination that increases expression of the viral RNA genome in the packaging cell line may be used. In the preferred embodiment the CMV enhancer/promoter sequence is used (U.S. Pat. No. 5,168,062; Karasuyama et al J. Exp. Med. 169:13 (1989).

The viral construct also comprises a transgene. The transgene may be any nucleotide sequence, including sequences that serve as markers for the provirus. Preferably the transgene comprises one or more RNA coding regions and/or one or more genes of interest. Schematic diagrams of exemplary retroviral constructs are shown in FIGS. 1A and 1B.

In the preferred embodiment the transgene comprises at least one RNA coding region. Preferably the RNA coding region is a DNA sequence that can serve as a template for the expression of a desired RNA molecule in the host cell. In one embodiment, the viral construct comprises two or more RNA coding regions.

The viral construct also preferably comprises at least one RNA Polymerase III promoter. The RNA Polymerase III promoter is operably linked to the RNA coding region and can also be linked to a termination sequence. In addition, more than one RNA Polymerase III promoter may be incorporated. RNA polymerase promoters are described herein, including supra.

In one embodiment the transgene comprises a gene of interest that encodes a protein that is desirably expressed in one or more cells of a transgenic animal, for example, a reporter or marker protein. Preferably the gene of interest is located between the 5′ LTR and 3′ LTR sequences. Further, the gene of interest is preferably in a functional relationship with other genetic elements, for example transcription regulatory sequences such as promoters and/or enhancers, to regulate expression of the gene of interest in a particular manner once the transgene is incorporated into the host genome. In certain embodiments, the useful transcriptional regulatory sequences are those that are highly regulated with respect to activity, both temporally and spatially.

Preferably the gene of interest is in a functional relationship with internal promoter and/or enhancer regulatory sequences, preferably with a polIII promoter and/or enhancer. An “internal” promoter/enhancer is one that is located between the 5′ LTR and the 3′ LTR sequences in the viral construct and is operably linked to the gene that is desirably expressed. The internal promoter/enhancer is preferably selected based on the desired expression pattern of the gene of interest and the specific properties of known promoters/enhancers. Thus, the internal promoter may be a constitutive promoter. Non-limiting examples of constitutive promoters that may be used include the promoter for ubiquitin, CMV (U.S. Pat. No. 5,168,062; Karasuyama et al J. Exp. Med. 169:13 (1989), Beta-actin (Gunning et al. Proc. Natl. Acad. Sci. USA 84:4831-4835 (1987) and pgk (see, for example, U.S. Pat. Nos. 4,615,974 and 5,104,795; Adra et al. Gene 60:65-74 (1987), Singer-Sam et al. Gene 32:409-417 (1984) and Dobson et al. Nucleic Acids Res. 10:2635-2637 (1982)). Alternatively, the promoter may be a tissue specific promoter. Several non-limiting examples of tissue specific promoters that may be used include Ick (see, for example, Garvin et al. Mol. Cell. Biol. 8:3058-3064 (1988) and Takadera et al. Mol. Cell. Biol. 9:2173-2180 (1989)), myogenin (Yee et al. Genes and Development 7:1277-1289 (1993), and thyl (Gundersen et al. Gene 113:207-214 (1992). In addition, promoters may be selected to allow for inducible expression of the transgene. A number of systems for inducible expression using such a promoter are known in the art, including the tetracycline responsive system and the lac operator-repressor system. It is also contemplated that a combination of promoters may be used to obtain the desired expression of the gene of interest.

An internal enhancer may also be present in the viral construct to increase expression of the gene of interest. For example the CMV enhancer (Karasuyama et al J. Exp. Med. 169:13 (1989) may be used in combination with the chicken beta-actin promoter (see, e.g., JP 1990005890-A1). Again, one of skill in the art will be able to select the appropriate enhancer based on the desired expression pattern.

The gene of interest is not limited in any way and includes any gene that the skilled practitioner desires to have integrated and/or expressed in, e.g., a cell or an animal. For example, the gene of interest may be one that encodes a protein that serves as a marker to identify cells comprising the provirus. In other embodiments the gene of interest encodes a protein that modifies a physical characteristic of a cell or transgenic animal, such as a protein that modifies size, growth, or tissue composition. In another example the gene of interest may encode a protein of commercial value that may be harvested from a cell or transgenic animal

In addition, more than one gene of interest may be placed in functional relationship with the internal promoter. For example a gene encoding a marker protein may be placed after the primary gene of interest to allow for identification of cells that are expressing the desired protein. In one embodiment a fluorescent marker protein, preferably green fluorescent protein (GFP), is incorporated into the construct along with the gene of interest. If a second marker gene is included, an internal ribosomal entry site (IRES) sequence may preferably included (U.S. Pat. No. 4,937,190). The IRES sequence may facilitate the expression of the reporter gene. Alternatively, the second marker gene may be placed in an intron. Introns are described herein, including supra.

The viral construct may also contain additional genetic elements. The types of elements that may be included in the construct are not limited in any way and will be chosen by the skilled practitioner to achieve a particular result. For example, a signal that facilitates nuclear entry of the viral genome in the target cell may be included. An example of such a signal is the HIV-1 flap signal.

Further, elements may be included that facilitate the characterization of the provirus integration site in the genome of the animal. For example, a tRNA amber suppressor sequence may be included in the construct.

In addition, the construct may contain one or more genetic elements designed to enhance expression of the gene of interest. For example, a woodchuck hepatitis virus responsive element (WRE) may be placed into the construct (Zufferey et al. J. Virol 74:3668-3681 (1999); Deglon et al. Hum. Gene Ther. 11: 179-190 (2000)).

A chicken beta-globin insulator (Chung et al. Proc. Natl. Acad. Sci. USA 94:575-580 (1997)) may also be included in the viral construct. This element has been shown to reduce the chance of silencing the integrated provirus in the transgenic animal due to methylation and heterochromatinization effects. In addition, the insulator may shield the internal enhancer, promoter and exogenous gene from positive or negative positional effects from surrounding DNA at the integration site on the chromosome.

Any additional genetic elements are preferably inserted 3′ of the gene of interest.

In a specific embodiment, the viral vector comprises: a cytomegalovirus (CMV) enhancer/promoter sequence; the R and U5 sequences from the HIV 5′ LTR; the HIV-1 flap signal; an internal enhancer; an internal promoter; a gene of interest; the woodchuck hepatitis virus responsive element; a tRNA amber suppressor sequence; a U3 element with a deletion of its enhancer sequence; the chicken beta-globin insulator; and the R and U5 sequences of the 3′ HIV LTR.

The viral construct is preferably cloned into a plasmid that may be transfected into a packaging cell line. The preferred plasmid preferably comprises sequences useful for replication of the plasmid in bacteria.

Any method known in the art may be used to produce infectious retroviral particles whose genome comprises an RNA copy of the viral construct described above.

Preferably, the viral construct is introduced into a packaging cell line. The packaging cell line provides the viral proteins that are required in trans for the packaging of the viral genomic RNA into viral particles. The packaging cell line may be any cell line that is capable of expressing retroviral proteins. Preferred packaging cell lines include 293 (ATCC CCL X), HeLa (ATCC CCL 2), D17 (ATCC CCL 183), MDCK (ATCC CCL 34), BHK (ATCC CCL-10) and Cf2Th (ATCC CRL 1430). The most preferable cell line is the 293 cell line.

The packaging cell line may stably express the necessary viral proteins. Such a packaging cell line is described, for example, in U.S. Pat. No. 6,218,181. Alternatively a packaging cell line may be transiently transfected with plasmids comprising nucleic acid that encodes the necessary viral proteins.

In one embodiment a packaging cell line that stably expresses the viral proteins required for packaging the RNA genome is transfected with a plasmid comprising the viral construct described above.

In another embodiment a packaging cell line that does not stably express the necessary viral proteins is co-transfected with two or more plasmids essentially as described in Yee et al. (Methods Cell. Biol. 43A, 99-112 (1994)). One of the plasmids comprises the viral construct comprising the transgene. The other plasmid(s) comprises nucleic acid encoding the proteins necessary to allow the cells to produce functional virus that is able to infect the desired host cell.

The packaging cell line may not express envelope gene products. In this case the packaging cell line will package the viral genome into particles that lack an envelope protein. As the envelope protein is responsible, in part, for the host range of the viral particles, the viruses are preferably pseudotyped. Thus the packaging cell line is preferably transfected with a plasmid comprising sequences encoding a membrane-associated protein that will permit entry of the virus into a host cell. One of skill in the art will be able to choose the appropriate pseudotype for the host cell that is to be used. For example, in one embodiment the viruses are pseudotyped with the vesicular stomatitis virus envelope glycoprotein (VSVg). In addition to conferring a specific host range this pseudotype may permit the virus to be concentrated to a very high titer. Viruses can alternatively be pseudotyped with ecotropic envelope proteins that limit infection to a specific species, such as mice or birds. For example, in another embodiment, a mutant ecotropic envelope protein is used, such as the ecotropic envelope protein 4.17 (Powell et al. Nature Biotechnology 18(12):1279-1282 (2000)).

In the preferred embodiment a packaging cell line that does not stably express viral proteins is transfected with the viral construct, a second vector comprising the HIV-1 packaging vector with the env, nef, 5′LTR, 3′LTR and vpu sequences deleted, and a third vector encoding an envelope glycoprotein. Preferably the third vector encodes the VSVg envelope glycoprotein.

In another embodiment of invention, RNA interference activity of the packaging cells is suppressed to improve the production of recombinant virus. This includes, without limitation, the use of cotransfection or stable transfection of constructs expressing double-stranded RNA complex molecules to inhibit Dicer, an RNase III family member of ribonuclease which is essential for RNA interference (Hammond et al. Nat. Rev. Genet. 2:110-119 (2001)).

The recombinant virus is then preferably purified from the packaging cells, titered and diluted to the desired concentration.

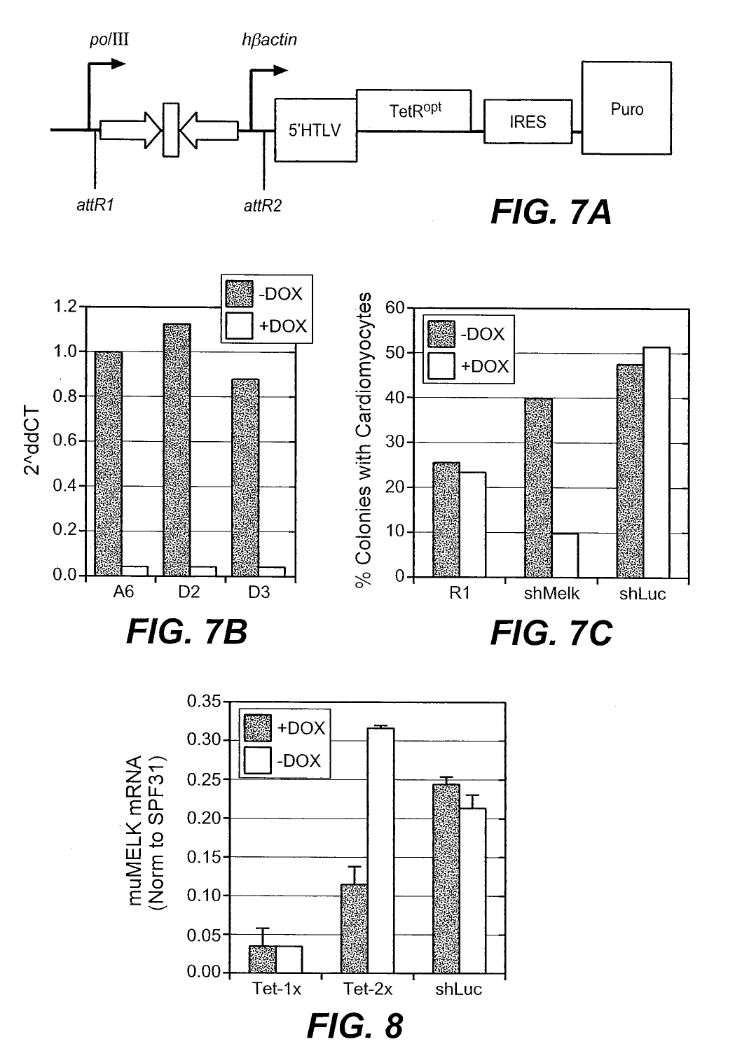

The vectors of the invention can be used to generate transgenic mammals. A vector suitable for inducible delivery of a RNA coding sequence is described and exemplified herein, e.g., in FIG. 7A. In some embodiments, the vector is a retroviral vector. Retroviral vectors suitable for generation of transgenic mammals are described and exemplified herein, e.g., in FIGS. 1A, 9A, 16A and B, and 10A.

Embryonic stem cells are derived from early mammalian embryos and display characteristics of totipotency, such that subsequent to being transferred to a suitable in viva environment these cells contribute to the primary germ layers, ectoderm, endoderm, and mesoderm, and populate the germline of mice as described by Evans and Kaufman, Nature 292, 154-156 (1981) and Martin, Proc. Natl. Acad. Sci. USA 78, 7634-7638 (1981), both of which are incorporated herein by reference. Embryonic stem cells can be propagated in an undifferentiated state and genetically manipulated in vitro. A transgenic non human mammal can be generated by introducing a vector of the invention into embryonic stem cells, followed by transplantation of the embryonic stem cells into embryos thereby effecting germ-line transmission. In some embodiments, the vector comprises a RNA coding region operably linked to an inducible promoter, and the vector is capable of inhibiting the expression of a target gene in the ES cell or transgenic mammal.

An embryonic stem cell of the invention that has been transduced or transfected with a vector can be stably propagated through undifferentiated proliferation. An embryonic stem cell of the invention further can be isolated from a cell line or derived directly from an embryo prior to transduction with the invention vector carrying, e.g., a double-stranded RNA complex transgene. In vitro differentiation of the embryonic stem cells can be studied by culturing of embryonic stem cells in aggregates that form embryoid bodies (EBs). The methods provided by the invention allow for stable expression of a vector expressing, e.g., a double-stranded RNA complex transgene formed by a nucleic acid sequence derived from a target gene transcript and its reverse complement and, consequently, provide the capability of inhibiting target gene expression.

An embryonic stem cell of the invention that has been transduced or transfected by a vector of the invention that is, e.g., capable of inhibiting the expression of a target gene, can be cultivated in hanging drops for a time appropriate to allow formation of an embryonic body. The inhibition of expression of a target gene can be evaluated in cells isolated from the embryoid body. In one embodiment of the invention, a non human embryonic stem cell comprising a vector of the invention capable of inhibiting the expression of a target gene is injected into a non-human mammal to derive a tissue consisting of cells in which the target gene is inhibited. For example, a triatoma can be induced by injecting a suspension of non-human embryonic stem cells into a host mammal, for example, a mouse, rat, dog, cow or monkey. Upon tumor formation, fragments of the tissue can be removed and evaluated for the effects of target gene inhibition. The vectors provided by the invention allow for generation of embryonic stem cells comprising the stable expression of a double-stranded RNA complex capable of inhibiting target gene expression. The embryonic stem cells generated via the invention methods may participate in formation of all three germ layers and stably express the double-stranded RNA complex transgene during differentiation, allowing for sustained target gene inhibition via the invention method. If desired, a stable cell line can be established from a cell isolated from a tissue that is derived from an embryonic stem cell infected with a vector of the invention.

In another aspect, an oocyte or one or more embryonic cells are infected with the recombinant virus produced as described above. One of skill in the art will recognize that the method of infection and the treatment of the cell following infection will depend upon the type of animal from which the cell is obtained. For example, mammalian cells are preferably implanted in a pseudopregnant female following infection while for the generation of transgenic birds or fish, the virus is preferably delivered to a laid egg and thus implantation is not required.

While early methods of making transgenic animals required the cells to be rapidly dividing, there is no such requirement in the methods of the present invention. Thus the cell may be contacted at any point in development. In the preferred embodiment, a zygote is contacted with the recombinant virus.

The cells to be infected with the virus may be obtained by any method known in the art and appropriate for the specific species in which it is desired to make a transgenic animal. For example, the recovery of fertilized mouse oocytes is described in Hogan et al. (Manipulating the Mouse Embryo: A Laboratory Manual. 2^{nd} ed. Cold Spring Harbor Laboratory Press, NY (1994)). A method for obtaining fertilized rat oocytes is described in Armstrong et al. (Biol. Reprod. 39, 511-518 (1998)).

It is not necessary that the cells be contacted after fertilization. In one embodiment, the virus is delivered to unfertilized ova. Development may then be initialized, for example by in vitro fertilization.

The virus may be delivered to the cell in any way that allows the virus to infect the cell. Preferably the virus is allowed to contact the cell membrane. Two preferred methods of delivering the virus to mammalian cells, injection and direct contact, are described below.

In a first embodiment the virus is injected into the perivitelline space between the zona pellucida and the cell membrane of a single-cell zygote. Preferably less than 50 picoliters of viral suspension is injected, more preferably less than 25 picoliters and even more preferably about 10 picoliters.

The virus is preferably present in a viral suspension and may be injected by any method known in the art. The viral suspension is preferably injected through a hydraulic injector. More preferably a glass micropipette is used to inject the virus. In one embodiment a micropipette is prepared by pulling borosilicate glass capillary on a pipette puller. The lentiviral suspension may be loaded into the micropipette from the tip using gentle negative pressure.

In one embodiment the cell is stabilized with a holding pipette mounted on a micromanipulator, such as by gentle negative pressure against a fire-polished pipette, and a second micromanipulator is used to direct the tip of a micropipette into the space between the zona pellucida and the cell membrane, where the virus is injected.

In another embodiment the zona pellucida is removed from the cell to produce a denuded embryo and the cell membrane is contacted with the virus. The zona pellucida may be removed by any method known in the art. Preferably it is removed by enzymatic treatment. For example, treatment with pronase may be used to remove the zona pellucida while the cell membrane is kept intact. Alternatively, the cell may be placed in media at pH at which the zona pellucida dissolves while the cell membrane remains intact. For example the cell may be incubated in an acidic Tyrode's solution at room temperature for several minutes. Once the zona pellucida is removed, any method that allows for the virus to contact the cell membrane may be used. Preferably, the cell is incubated in a solution containing the virus. Even more preferably, the solution is media that facilitates survival of the cell.

In this embodiment, the cells are preferably contacted with the virus in culture plates. The virus may be suspended in media and added to the wells of a multi-well culture plate. The cells may then be plated in the individual wells. The media containing the virus may be added prior to the plating of the cells or after the cells have been plated. However, any amount of media may be used as long as an appropriate concentration of virus in the media is maintained such that infection of the host cell occurs.

The cells are preferably incubated with the virus for a sufficient amount of time to allow the virus to infect the cells. Preferably the cells are incubated with virus for at least 1 hour, more preferably at least 5 hours and even more preferably at least 10 hours.

Both the injection and direct contact embodiments may advantageously be scaled up to allow high throughput transgenesis. Because of the relative simplicity of the injection technique, it is possible to inject many embryos rapidly. For example, it is possible to inject more than 200 fertilized oocytes in less than one hour. With regard to the direct contact embodiment, any number of embryos may be incubated in the viral suspension simultaneously. This may be accomplished, for example, by planting the desired number of single-cell zygotes in multi-well tissue culture plates containing the virus suspended in media appropriate for the survival and growth of the cells.

Following infection with the virus, the cells are preferably implanted in an animal. More preferably cells infected with the virus are implanted in pseudo-pregnant animals of the same species from which the infected cells were obtained. Methods of creating pseudo-pregnancy in animals and implanting embryos are well known in the art and are described, for example, in Hogan et al. (Manipulating the Mouse Embryo: A Laboratory Manual. 2^{nd} ed. Cold Spring Harbor Laboratory Press, NY (1994)).

In the preferred embodiment early stage embryos (approximately 0-2.5 days p.c.) still with an intact zona pellucida are transferred to the oviduct of timed pseudopregnant female (preferably 0.5 days p.c.), while embryos that have reached the blastocyst stage are transferred to the uterus of timed pseudopregnant females (preferably 2.5 days p.c.). Denuded embryos are preferably cultured in vitro until they reach the morula or blastocyst stage (48 to 72 hours in culture), and are then implanted into appropriately timed pseudopregnant females.

The embryos and resulting animals may be analyzed, for example for integration of the transgene, the number of copies of the transgene that integrated, the location of the integration, the ability to transmit the transgene to progeny and expression of the transgene. Such analysis may be carried out at any time and may be carried out by any methods known in the art. Standard techniques are described, for example, in Hogan et al. (supra).

The methods of infecting cells disclosed above do not depend upon species-specific characteristics of the cells. As a result, they are readily extended to all mammalian species.

As discussed above, the modified retrovirus can be pseudotyped to confer upon it a broad host range. One of skill in the art would also be aware of appropriate internal promoters to achieve the desired expression of a gene of interest in a particular animal species. Thus, one of skill in the art will be able to modify the method of infecting cells to create transgenic animals of any species.

The invention also provides methods using the polynucleotides of the invention. Some of the methods described herein allow the expression of RNA coding region in cells, and are particularly suited to the expression of small RNA molecules which are preferably expressed from a Pol III promoter, as well as microRNA molecules which may be expressed from a Pol II promoter. The ability to down-regulate a target gene has many therapeutic and research applications, including identifying the biological functions of particular genes. Assays that can be used for understanding the biological effects of knocking down a target gene include cell based assays, enzymatic assays, array analysis. Double-stranded RNA complexes exert their effects at the mRNA level. The simplest assay for RNA interference validation and transfection optimization relies on qRT-PCR to measure target transcript levels in gene specific double-stranded RNA complex-treated cells versus negative control treated cells. Applied Biosystems' TaqMan® Gene Expression Assays, available for >41,000 human, mouse, and rat genes, are also useful for this purpose. The extent of knockdown at the protein level can also be assessed. Since native protein is recovered in most cases, enzymatic assays can also be performed. Double-stranded RNA complex, target mRNA, and target protein levels can be correlated. For example, in a method for screening; an agent for the ability to restore or modulate the effect of target gene inhibition by adding an agent to an appropriate cell line or introducing the agent into a transgenic non-human mammal or into a cell line in which the expression of a target gene is inhibited.

Generally a plurality of assay mixtures are run in parallel with different agent concentrations to obtain a differential response to the various concentrations. Typically, one of these concentrations serves as a negative control, i.e. at zero concentration or below the level of detection. Agents can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of naturally-occurring agents in the form of bacterial, fungal, plant and animal extracts are available or readily produced. Additionally, natural or synthetically produced libraries and compounds are readily modified through conventional chemical, physical and biochemical means, and can be used to produce combinatorial libraries. Known pharmacological agents can be subjected to directed or random chemical modifications, such as acylation, alkylation, esterification, acidification, to produce structural analogs.

In a related embodiment, the vectors of the invention also can be used in high-throughput in vitro screens for loss-of-function phenotypes. If desired, a population of cells can be prepared that are transduced with a library of lentiviral vectors encompassing different double-stranded RNA complexes. Upon transduction, the cells can be screened for a particular phenotype of interest. In this embodiment, the double-stranded-RNA complex can be used to identify genes that, upon inhibition, elicit a particular phenotype indicating their involvement in a process/condition of interest. It is understood that in this embodiment the target gene can be either randomly selected or can be chosen semi-randomly, for example, based on a microarray analysis of the cells chosen for transduction. A cell type of interest can be selected such as, for example, embryonic stem cells, pancreatic cells, cancer cells, and a library of lentiviral vectors can be prepared that encompass nucleic acid sequences selected based on a microarray analysis performed on the particular cell type. One skilled in the art will be able to select a particular cell type that is known to express the particular genes of interest to be studied, for example, pancreatic cells to screen for genes involved in diabetes.

For example, a method for screening; an agent for the ability to restore or modulate the effect of target gene inhibition by adding an agent to an appropriate cell line or introducing the agent into a transgenic non-human mammal or into a cell line in which the expression of a target gene is inhibited. Transgenic animals or cells in which the expression of a target gene is inhibited as well as cell lines generated according to this invention can be used in these methods.

A retroviral vector of the invention capable of inhibiting the expression of at least one target gene also is useful in therapeutic applications designed to inactivate disease-associated transcripts and thereby reduce the severity of inherited metabolic, infectious or malignant conditions. The therapeutic applications of the invention can be used to reduce the severity of dominant genetic conditions, including those caused by a point mutation, by inhibiting the expression of the mutant allele while leaving unaffected the expression of the remaining wild-type transcript. This embodiment of the invention capitalizes on the sequence specificity of siRNA which requires perfect match for target gene inhibition. Any condition that can be reduced in severity by decreasing the expression of a gene product can be appropriate for the screening and therapeutic methods of the invention including, for example, cancer, hemophilia, diabetes, Alzheimer's disease as well as triplet repeat expansion diseases including fragile X syndrome, Huntington's chorea, myotonic muscular dystrophy, spinocerebellar atrophy, Friedreich ataxia, dentatorubral and pallidoluysian atrophy, and Machado-Joseph disease.

For ex vivo therapy applications using retroviral vectors of the invention, cells are removed from a subject and cultured in vitro. The RNA transcript is introduced into the cells in vitro via transduction or transfection with a retroviral vector of the invention and subsequently the modified cells are expanded in culture followed by reimplantation into the subject.

B. TASK Binding Oligopeptides

TASK binding oligopeptides of the present invention are oligopeptides that bind, preferably specifically, to a TASK polypeptide as described herein. TASK binding oligopeptides may be chemically synthesized using known oligopeptide synthesis methodology or may be prepared and purified using recombinant technology. TASK binding oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such oligopeptides that are capable of binding, preferably specifically, to a TASK polypeptide as described herein. TASK binding oligopeptides may be identified without undue experimentation using well Known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

In this regard, bacteriophage (phage) display is one well known technique which allows one to screen large oligopeptide libraries to identify member(s) of those libraries which are capable of specifically binding to a polypeptide target. Phage display is a technique by which variant polypeptides are displayed as fusion proteins to the coat protein on the surface of bacteriophage particles (Scott, J. K. and Smith, G. P. (1990) Science 249: 386). The utility of phage display lies in the fact that large libraries of selectively randomized protein variants (or randomly cloned cDNAs) can be rapidly and efficiently sorted for those sequences that bind to a target molecule with high affinity. Display of peptide (Cwirla, S. E. et al. (1990) Proc. Natl. Acad. Sci. USA, 87:6378) or protein (Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363) libraries on phage have been used for screening millions of polypeptides or oligopeptides for ones with specific binding properties (Smith, G. P. (1991) Current Opin. Biotechnol., 2:668). Sorting phage libraries of random mutants requires a strategy for constructing and propagating a large number of variants, a procedure for affinity purification using the target receptor, and a means of evaluating the results of binding enrichments. U.S. Pat. Nos. 5,223,409, 5,403,484, 5,571,689, and 5,663,143.

Although most phage display methods have used filamentous phage, lambdoid phage display systems (WO 95/34683; U.S. Pat. No. 5,627,024), T4 phage display systems (Ren, Z-J. et al. (1998) Gene 215:439; Zhu, Z. (1997) CAN 33:534; Jiang, J. et al. (1997) can 128:44380; Ren, Z-J. et al. (1997) CAN 127:215644; Ren, Z-J. (1996) Protein Sci. 5:1833; Efimov, V. P. et al. (1995) Virus Genes 10: 173) and T7 phage display systems (Smith, G. P. and Scott, J. K. (1993) Methods in Enzymology, 217, 228-257; U.S. Pat. No. 5,766,905) are also known.

Many other improvements and variations of the basic phage display concept have now been developed. These improvements enhance the ability of display systems to screen peptide libraries for binding to selected target molecules and to display functional proteins with the potential of screening these proteins for desired properties. Combinatorial reaction devices for phage display reactions have been developed (WO 98/14277) and phage display libraries have been used to analyze and control bimolecular interactions (WO 98/20169; WO 98/20159) and properties of constrained helical peptides (WO 98/20036). WO 97/35196 describes a method of isolating an affinity ligand in which a phage display library is contacted with one solution in which the ligand will bind to a target molecule and a second solution in which the affinity ligand will not bind to the target molecule, to selectively isolate binding ligands. WO 97/46251 describes a method of biopanning a random phage display library with an affinity purified antibody and then isolating binding phage, followed by a micropanning process using microplate wells to isolate high affinity binding phage. The use of Staphlylococcus aureus protein A as an affinity tag has also been reported (Li et al. (1998) Mol. Biotech., 9:187). WO 97/47314 describes the use of substrate subtraction libraries to distinguish enzyme specificities using a combinatorial library which may be a phage display library. A method for selecting enzymes suitable for use in detergents using phage display is described in WO 97/09446. Additional methods of selecting specific binding proteins are described in U.S. Pat. Nos. 5,498,538, 5,432,018, and WO 98/15833.

Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

TASK peptides may also be expressed through an inducible system. The current invention provides for pHUSH-ProEx, an inducible selectable vector system. pHUSH-ProEx can also be packaged into active viral particles. Utility to pHUSH-ProEx can be found by combining it with TASK oligopeptides of the invention or useful fragments of a TASK polypeptide, and expressing either TASK fragments or TASK oligopeptides in such a manner as to inhibit the effect that a TASK polypeptide or fragment thereof has on cell proliferation.

C. TASK Binding Small Molecules

TASK binding small molecules are small molecules other than oligopeptides or antibodies as defined herein that bind, preferably specifically, to a TASK polypeptide as described herein. TASK binding small molecules may be identified and chemically synthesized using known methodology (see, e.g., PCT Publication Nos. WO00/00823 and WO00/39585). TASK binding small molecules are usually about 500 daltons in size, alternatively less than about 1500, 750, 500, 250 or 200 daltons in size, wherein such small molecules that are capable of binding, preferably specifically, to a TASK polypeptide as described herein may be identified without undue experimentation using well known techniques. In this regard, it is noted that techniques for screening small molecule libraries for molecules that are capable of binding to a polypeptide target are well known in the art (see, e.g., PCT Publication Nos. WO00/00823 and WO00/39585). TASK binding small molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

D. Screening for TASK Binding Oligopeptides, TASK Binding Small Molecules and TASK-Directed Double-Stranded RNA Complexes with the Desired Properties

Techniques for generating antibodies, double-stranded RNA complexes and small molecules that bind to TASK polypeptides have been described above. One may further select antibodies, double-stranded RNA complexes or other small molecules with certain biological characteristics, as desired.

The growth inhibitory effects of a double-stranded RNA complex or other small molecule of the invention may be assessed by methods known in the art, e.g., using cells which express a TASK polypeptide either endogenously or following transfection with the TASK gene. For example, appropriate tumor cell lines and TASK-transfected cells may be treated with an TASK double-stranded RNA complex or other small molecule of the invention at various concentrations for a few days (e.g., 2-7) days and stained with crystal violet or MTT or analyzed by some other colorimetric assay. Another method of measuring proliferation would be by comparing ³H-thymidine uptake by the cells treated in the presence or absence an TASK double-stranded RNA complex or TASK binding small molecule of the invention. After treatment, the cells are harvested and the amount of radioactivity incorporated into the DNA quantitated in a scintillation counter. Appropriate positive controls include treatment of a selected cell line with a growth inhibitory antibody known to inhibit growth of that cell line. Growth inhibition of tumor cells in vivo can be determined in various ways known in the art. Preferably, the tumor cell is one that overexpresses a TASK polypeptide. Preferably, the TASK double-stranded RNA complex or TASK binding small molecule will inhibit cell proliferation of a TASK-expressing tumor cell in vitro or in vivo by about 25-100% compared to the untreated tumor cell, more preferably, by about 30-100%, and even more preferably by about 50-100% or 70-100%. To select for an TASK double-stranded RNA complex or TASK binding small molecule which induces cell death, loss of membrane integrity as indicated by, e.g., propidium iodide (PI), trypan blue or 7AAD uptake may be assessed relative to control. A PI uptake assay can be performed in the absence of complement and immune effector cells. TASK polypeptide-expressing tumor cells are incubated with medium alone or medium containing the appropriate TASK double-stranded RNA complex or TASK binding small molecule. The cells are incubated for approximately a 3 day time period. Following each treatment, cells are washed and aliquoted into 35 mm strainer-capped 12×75 tubes (1 ml per tube, 3 tubes per treatment group) for removal of cell clumps. Tubes then receive PI (10 μg/ml). Samples may be analyzed using a FACSCAN® flow cytometer and FACSCONVERT® CellQuest software (Becton Dickinson). Those TASK double-stranded RNA complex or TASK binding small molecules that induce statistically significant levels of cell death as determined by PI uptake may be selected as cell death-inducing TASK double-stranded RNA complex or TASK binding small molecules.

To screen for oligopeptides or other small molecules which bind to an epitope on a TASK polypeptide bound by an antibody of interest, a routine cross-blocking assay such as that described in Antibodies, A Laboratory Manual, Cold Spring Harbor Laboratory, Ed Harlow and David Lane (1988), can be performed. This assay can be used to determine if a test oligopeptide or other small molecule binds the same site or epitope as a known anti-TASK antibody

E. Full-Length TASK Polypeptides

The present invention also provides newly identified and isolated nucleotide sequences encoding polypeptides referred to in the present application as TASK polypeptides. In particular, cDNAs (partial and full-length) encoding various TASK polypeptides have been identified and isolated, as disclosed in further detail in the Examples below.

As disclosed in the Examples below, various cDNA clones have been described. The predicted amino acid sequence can be determined from the nucleotide sequence using routine skill. For the TASK polypeptides and encoding nucleic acids described herein, in some cases, Applicants have identified what is believed to be the reading frame best identifiable with the sequence information available at the time.

F. TASK Polypeptide Variants

In addition to the full-length native sequence TASK polypeptides described herein, it is contemplated that TASK polypeptide variants can be prepared. TASK polypeptide variants can be prepared by introducing appropriate nucleotide changes into the encoding DNA, and/or by synthesis of the desired polypeptide. Those skilled in the art will appreciate that amino acid changes may alter post-translational processes of the TASK polypeptide, such as changing the number or position of glycosylation sites or altering the membrane anchoring characteristics.

Variations in the TASK polypeptides described herein, can be made, for example, using any of the techniques and guidelines for conservative and non-conservative mutations set forth, for instance, in U.S. Pat. No. 5,364,934. Variations may be a substitution, deletion or insertion of one or more codons encoding the polypeptide that results in a change in the amino acid sequence as compared with the native sequence polypeptide. Optionally the variation is by substitution of at least one amino acid with any other amino acid in one or more of the domains of the TASK polypeptide. Guidance in determining which amino acid residue may be inserted, substituted or deleted without adversely affecting the desired activity may be found by comparing the sequence of the TASK polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

TASK polypeptide fragments are provided herein. Such fragments may be truncated at the N-terminus or C-terminus, or may lack internal residues, for example, when compared with a full length native protein. Certain fragments lack amino acid residues that are not essential for a desired biological activity of the TASK polypeptide.

TASK polypeptide fragments may be prepared by any of a number of conventional techniques. Desired peptide fragments may be chemically synthesized. An alternative approach involves generating polypeptide fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, TASK polypeptide fragments share at least one biological and/or immunological activity with the native TASK polypeptide disclosed herein.

In particular embodiments, conservative substitutions of interest are shown in Table 6 under the heading of preferred substitutions. If such substitutions result in a change in biological activity, then more substantial changes, denominated exemplary substitutions in Table 6, or as further described below in reference to amino acid classes, are introduced and the products screened.

TABLE 6

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in function or immunological identity of the TASK polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;
(2) neutral hydrophilic: cys, ser, thr;
(3) acidic: asp, glu;
(4) basic: asn, gin, his, lys, arg;
(5) residues that influence chain orientation: gly, pro; and
(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the TASK polypeptide variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244:1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

Any cysteine residue not involved in maintaining the proper conformation of the TASK polypeptide also may be substituted, generally with serine, to improve the oxidative stability of the molecule and prevent aberrant crosslinking. Conversely, cysteine bond(s) may be added to the TASK polypeptide to improve its stability.

G. Modifications of Anti-TASK Antibodies and TASK Polypeptides

Covalent modifications of TASK polypeptides are included within the scope of this invention. One type of covalent modification includes reacting targeted amino acid residues of a TASK polypeptide with an organic derivatizing agent that is capable of reacting with selected side chains or the N- or C-terminal residues of the TASK polypeptide. Derivatization with bifunctional agents is useful, for instance, for crosslinking the TASK polypeptide to a water-insoluble support matrix or surface for use in the method for purifying TASK small molecules. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the TASK polypeptide included within the scope of this invention comprises altering the native glycosylation pattern of the polypeptide. “Altering the native glycosylation pattern” is intended for purposes herein to mean deleting one or more carbohydrate moieties found in native sequence TASK polypeptide (either by removing the underlying glycosylation site or by deleting the glycosylation by chemical and/or enzymatic means), and/or adding one or more glycosylation sites that are not present in the native sequence TASK polypeptide. In addition, the phrase includes qualitative changes in the glycosylation of the native proteins, involving a change in the nature and proportions of the various carbohydrate moieties present.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threomine, although 5-hydroxyproline or 5-hydroxylysine may also be used.

Addition of glycosylation sites to the TASK polypeptide is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original TASK polypeptide (for O-linked glycosylation sites). The TASK polypeptide amino acid sequence may optionally be altered through changes at the DNA level, particularly by mutating the DNA encoding the TASK polypeptide at preselected bases such that codons are generated that will translate into the desired amino acids.

Another means of increasing the number of carbohydrate moieties on the TASK polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the TASK polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of the TASK polypeptide comprises linking the polypeptide to one of a variety of nonproteinaceous polymers, e.g., polyethylene glycol (PEG), polypropylene glycol, or polyoxyalkylenes, in the manner set forth in U.S. Pat. No. 4,640,835; 4,496,689; 4,301,144; 4,670,417; 4,791,192 or 4,179,337. The polypeptide also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

The TASK polypeptide of the present invention may also be modified in a way to form chimeric molecules comprising a TASK polypeptide fused to another, heterologous polypeptide or amino acid sequence.

In one embodiment, such a chimeric molecule comprises a fusion of the TASK polypeptide with a tag polypeptide which provides an epitope to which an anti-tag antibody can selectively bind. The epitope tag is generally placed at the amino- or carboxyl-terminus of the TASK polypeptide. The presence of such epitope-tagged forms of the TASK polypeptide can be detected using an antibody against the tag polypeptide. Also, provision of the epitope tag enables the TASK polypeptide to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In an alternative embodiment, the chimeric molecule may comprise a fusion of the TASK polypeptide with an immunoglobulin or a particular region of an immunoglobulin. For a bivalent form of the chimeric molecule (also referred to as an “immunoadhesin”), such a fusion could be to the Fc region of an IgG molecule. In a particularly preferred embodiment, the immunoglobulin fusion includes the hinge, CH₂ and CH₃, or the hinge, CH₁, CH₂ and CH₃ regions of an IgG1 molecule. For the production of immunoglobulin fusions see also U.S. Pat. No. 5,428,130 issued Jun. 27, 1995.

H. Preparation of TASK Polypeptides

The description below relates primarily to production of TASK polypeptides by culturing cells transformed or transfected with a vector containing TASK polypeptide-encoding nucleic acid. It is, of course, contemplated that alternative methods, which are well known in the art, may be employed to prepare TASK polypeptides. For instance, the appropriate amino acid sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the TASK polypeptide may be chemically synthesized separately and combined using chemical or enzymatic methods to produce the desired TASK polypeptide.

1. Isolation of DNA Encoding TASK Polypeptide

DNA encoding TASK polypeptide may be obtained from a cDNA library prepared from tissue believed to possess the TASK polypeptide mRNA and to express it at a detectable level. Accordingly, human TASK polypeptide DNA can be conveniently obtained from a cDNA library prepared from human tissue. The TASK polypeptide-encoding gene may also be obtained from a genomic library or by known synthetic procedures (e.g., automated nucleic acid synthesis).

Libraries can be screened with probes (such as oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding the TASK polypeptide is to use PCR methodology [Sambrook et al., supra; Dieffenbach et al., PCR Primer: A Laboratory Manual (Cold Spring Harbor Laboratory Press, 1995)].

Techniques for screening a cDNA library are well known in the art. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for TASK polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCh, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3110 strain 37D6, which has the complete genotype tonA ptr3 phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for TASK polypeptide-encoding vectors. Saccharomyces cerevisiae is a commonly used lower eukaryotic host microorganism. Others include Schizosaccharomzyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharonyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated TASK polypeptide are derived from multicellular organisms. Examples of invertebrate cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for TASK polypeptide production and cultured in conventional nutrient media modified as appropriate for inducing promoters, selecting transformants, or amplifying the genes encoding the desired sequences.

3. Selection and Use of a Replicable Vector

The nucleic acid (e.g., cDNA or genomic DNA) encoding the TASK polypeptide may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The TASK may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the polypeptide. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 21 plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the TASK polypeptide-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene, 10: 157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the TASK polypeptide-encoding nucleic acid sequence to direct mRNA synthesis. Promoters recognized by a variety of potential host cells are well known. Promoters suitable for use with prokaryotic hosts include the p-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgamo (S.D.) sequence operably linked to the DNA encoding TASK polypeptide.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

TASK polypeptide transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the TASK polypeptide by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the TASK polypeptide coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the TASK polypeptide.

Still other methods, vectors, and host cells suitable for adaptation to the synthesis of the TASK polypeptide in recombinant vertebrate cell culture are described in Gething et al., Nature, 293:620-625 (1981); Mantei et al., Nature, 281:40-46 (1979); EP 117,060; and EP 117,058.

4. Culturing the Host Cells

The host cells used to produce the TASK polypeptide of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. No. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

5. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence TASK polypeptide or against a synthetic peptide based on the DNA sequences provided herein or against exogenous sequence fused to TASK DNA and encoding a specific antibody epitope.

6. Purification of TASK Polypeptide

Cells employed in expression of TASK polypeptide can be disrupted by various physical or chemical means, such as freeze-thaw cycling, sonication, mechanical disruption, or cell lysing agents.

It may be desired to purify the TASK polypeptide from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the TASK polypeptide. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular TASK polypeptide produced.

I. Pharmaceutical Formulations

Therapeutic formulations of the TASK binding oligopeptides, TASK double-stranded RNA complex, TASK binding small molecules and/or TASK polypeptides used in accordance with the present invention are prepared for storage by mixing the polypeptide, oligopeptide, double-stranded RNA complex or small molecule having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as acetate, Tris, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g., Zn-protein complexes); and/or non-ionic surfactants such as TWEEN®, PLURONICS® or polyethylene glycol (PEG).

The formulations herein may also contain more than one active compound as necessary for the particular indication being treated, preferably those with complementary activities that do not adversely affect each other. For example, in addition to a TASK binding oligopeptide, TASK double-stranded RNA complex, or TASK binding small molecule, it may be desirable to include in the one formulation, an additional double-stranded RNA complex, e.g., a second TASK double-stranded RNA complex which binds a different area on the TASK nucleic acid, or to some other target such as a growth factor that affects the growth of the particular cancer. Alternatively, or additionally, the composition may further comprise a chemotherapeutic agent, cytotoxic agent, cytokine, growth inhibitory agent, anti-hormonal agent, and/or cardioprotectant. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980).

Sustained-release preparations may be prepared. Suitable examples of sustained-release preparations include semi-permeable matrices of solid hydrophobic polymers containing the antibody or polypeptide, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

J. Diagnosis and Treatment with TASK double-stranded RNA complex and TASK Small Molecules

To determine TASK expression in the cancer, various diagnostic assays are available. In one embodiment, TASK polypeptide overexpression may be analyzed by immunohistochemistry (IHC). Parrafin embedded tissue sections from a tumor biopsy may be subjected to the IHC assay and accorded a TASK protein staining intensity criteria as follows:

Score 0— no staining is observed or membrane staining is observed in less than 10% of tumor cells.

Score 1+—a faint/barely perceptible staining is detected in more than 10% of the tumor cells. The cells are only stained in part of their membrane.

Score 2+—a weak to moderate complete staining is observed in more than 10% of the tumor cells.

Score 3+—a moderate to strong complete staining is observed in more than 10% of the tumor cells.

Those tumors with 0 or 1+ scores for TASK polypeptide expression may be characterized as not overexpressing TASK, whereas those tumors with 2+ or 3+scores may be characterized as overexpressing TASK.

Alternatively, or additionally, FISH assays such as the INFORM® (sold by Ventana, Arizona) or PATHVISION® (Vysis, Illinois) may be carried out on formalin-fixed, paraffin-embedded tumor tissue to determine the extent (if any) of TASK overexpression in the tumor.

TASK overexpression or amplification may be evaluated using an in vivo diagnostic assay, e.g., by administering a molecule (such as a double-stranded RNA complex, oligopeptide or small molecule) which binds the molecule to be detected and is tagged with a detectable label (e.g., a radioactive isotope or a fluorescent label) and externally scanning the patient for localization of the label.

As described above, the double-stranded RNA complexes and small molecules of the invention have various non-therapeutic applications. The double-stranded RNA complex and small molecules of the present invention can be useful for diagnosis and staging of TASK polypeptide-expressing cancers (e.g., in radioimaging). The oligopeptides and small molecules are also useful for purification or immunoprecipitation of TASK polypeptide from cells, for detection and quantitation of TASK polypeptide in vitro, e.g., in an ELISA or a Western blot, to kill and eliminate TASK-expressing cells from a population of mixed cells as a step in the purification of other cells.

Currently, depending on the stage of the cancer, cancer treatment involves one or a combination of the following therapies: surgery to remove the cancerous tissue, radiation therapy, and chemotherapy. double-stranded RNA complex or small molecule therapy may be especially desirable in elderly patients who do not tolerate the toxicity and side effects of chemotherapy well and in metastatic disease where radiation therapy has limited usefulness. The tumor targeting double-stranded RNA complex and small molecules of the invention are useful to alleviate TASK-expressing cancers upon initial diagnosis of the disease or during relapse. For therapeutic applications, the double-stranded RNA complex or small molecule can be used alone, or in combination therapy with, e.g., hormones, antiangiogens, or radiolabelled compounds, or with surgery, cryotherapy, and/or radiotherapy. Double-stranded RNA complex or small molecule treatment can be administered in conjunction with other forms of conventional therapy, either consecutively with, pre- or post-conventional therapy. Chemotherapeutic drugs such as TAXOTERE® (docetaxel), TAXOL® (palictaxel), estramustine and mitoxantrone are used in treating cancer, in particular, in good risk patients. In the present method of the invention for treating or alleviating cancer, the cancer patient can be administered double-stranded RNA complex or small molecule in conduction with treatment with the one or more of the preceding chemotherapeutic agents. In particular, combination therapy with palictaxel and modified derivatives (see, e.g., EP0600517) is contemplated. The double-stranded RNA complex or small molecule will be administered with a therapeutically effective dose of the chemotherapeutic agent. In another embodiment, the double-stranded RNA complex or small molecule is administered in conjunction with chemotherapy to enhance the activity and efficacy of the chemotherapeutic agent, e.g., paclitaxel. The Physicians' Desk Reference (PDR) discloses dosages of these agents that have been used in treatment of various cancers. The dosing regimen and dosages of these aforementioned chemotherapeutic drugs that are therapeutically effective will depend on the particular cancer being treated, the extent of the disease and other factors familiar to the physician of skill in the art and can be determined by the physician.

The double-stranded RNA complex, small molecules or toxin conjugates thereof are administered to a human patient, in accord with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. Intravenous or subcutaneous administration of the double-stranded RNA complex or organic molecule is preferred.

Other therapeutic regimens may be combined with the administration of the double-stranded RNA complex or small molecule. The combined administration includes co-administration, using separate formulations or a single pharmaceutical formulation, and consecutive administration in either order, wherein preferably there is a time period while both (or all) active agents simultaneously exert their biological activities. Preferably such combined therapy results in a synergistic therapeutic effect.

It may also be desirable to combine administration of the double-stranded RNA complex or small molecules with administration of an antibody directed against another tumor antigen associated with the particular cancer.

In another embodiment, the therapeutic treatment methods of the present invention involves the combined administration of a double-stranded RNA complex or small molecules and one or more chemotherapeutic agents or growth inhibitory agents, including co-administration of cocktails of different chemotherapeutic agents. Chemotherapeutic agents include estramustine phosphate, prednimustine, cisplatin, 5-fluorouracil, melphalan, cyclophosphamide, hydroxyurea and hydroxyureataxanes (such as paclitaxel and doxetaxel) and/or anthracycline antibiotics. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992).

The double-stranded RNA complex or small molecule may be combined with an anti-hormonal compound; e.g., an anti-estrogen compound such as tamoxifen; an anti-progesterone such as onapristone (see, EP 616 812); or an anti-androgen such as flutamide, in dosages known for such molecules. Where the cancer to be treated is androgen independent cancer, the patient may previously have been subjected to anti-androgen therapy and, after the cancer becomes androgen independent, the double-stranded RNA complex or small molecule (and optionally other agents as described herein) may be administered to the patient.

Sometimes, it may be beneficial to also co-administer a cardioprotectant (to prevent or reduce myocardial dysfunction associated with the therapy) or one or more cytokines to the patient. In addition to the above therapeutic regimes, the patient may be subjected to surgical removal of cancer cells and/or radiation therapy, before, simultaneously with, or post double-stranded RNA complex or small molecule therapy. Suitable dosages for any of the above co-administered agents are those presently used and may be lowered due to the combined action (synergy) of the agent and double-stranded RNA complex or small molecule.

For the prevention or treatment of disease, the dosage and mode of administration will be chosen by the physician according to known criteria. The appropriate dosage of double-stranded RNA complex or small molecule will depend on the type of disease to be treated, as defined above, the severity and course of the disease, whether the double-stranded RNA complex or small molecule is administered for preventive or therapeutic purposes, previous therapy, the patient's clinical history and response to the double-stranded RNA complex, oligopeptide or small molecule, and the discretion of the attending physician. The double-stranded RNA complex or small molecule is suitably administered to the patient at one time or over a series of treatments. Preferably, the double-stranded RNA complex or small molecule is administered by intravenous infusion or by subcutaneous injections. A dosing regimen can comprise administering an initial loading dose followed by a weekly maintenance dose. However, other dosage regimens may be useful. For repeated administrations over several days or longer, depending on the condition, the treatment is sustained until a desired suppression of disease symptoms occurs. The progress of this therapy can be readily monitored by conventional methods and assays and based on criteria known to the physician or other persons of skill in the art.

There are two major approaches to getting the nucleic acid (optionally contained in a vector) into the patient's cells; in vivo and ex vivo. For ex vivo treatment, the patient's cells are removed, the nucleic acid is introduced into these isolated cells and the modified cells are administered to the patient either directly or, for example, encapsulated within porous membranes which are implanted into the patient (see, e.g., U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retroviral vector.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). For review of the currently known gene marking and gene therapy protocols see Anderson et al., Science 256:808-813 (1992). See also WO 93/25673 and the references cited therein.

The present double-stranded RNA complex and small molecules are useful for treating a TASK-expressing cancer or alleviating one or more symptoms of the cancer in a mammal. Such a cancer includes melanoma, prostate cancer, cancer of the urinary tract, lung cancer, breast cancer, colon cancer and ovarian cancer, more specifically, prostate adenocarcinoma, renal cell carcinomas, colorectal adenocarcinomas, lung adenocarcinomas, lung squamous cell carcinomas, and pleural mesothelioma. The cancers encompass metastatic cancers of any of the preceding. The double-stranded RNA complex or small molecule is able to bind to at least a portion of the cancer cells that express TASK polypeptide in the mammal. In a preferred embodiment the double-stranded RNA complex or small molecule is effective to destroy or kill TASK-expressing tumor cells or inhibit the growth of such tumor cells, in vitro or in vivo.

The invention provides a composition comprising a double-stranded RNA complex or small molecule of the invention, and a carrier. For the purposes of treating cancer, compositions can be administered to the patient in need of such treatment, wherein the composition can comprise one or more TASK double-stranded RNA complex. In a further embodiment, the compositions can comprise these double-stranded RNA complex or small molecules in combination with other therapeutic agents such as cytotoxic or growth inhibitory agents, including chemotherapeutic agents. The invention also provides formulations comprising a double-stranded RNA complex or small molecule of the invention, and a carrier. In one embodiment, the formulation is a therapeutic formulation comprising a pharmaceutically acceptable carrier.

The invention also provides methods useful for treating a TASK polypeptide-expressing cancer or alleviating one or more symptoms of the cancer in a mammal, comprising administering a therapeutically effective amount of a double-stranded RNA complex or small molecule to the mammal. The double-stranded RNA complex or small molecule therapeutic compositions can be administered short term (acute) or chronic, or intermittent as directed by physician. Also provided are methods of inhibiting the growth of, and killing a TASK polypeptide-expressing cell.

The invention also provides kits and articles of manufacture comprising at least one double-stranded RNA complex or small molecule. Kits containing double-stranded RNA complex or small molecules find use, e.g., for TASK cell killing assays, for purification or immunoprecipitation of TASK polypeptide from cells. For example, for isolation and purification of TASK, the kit can contain a double-stranded RNA complex or small molecule coupled to beads (e.g., sepharose beads). Kits can be provided which contain the antibodies, oligopeptides or organic molecules for detection and quantitation of TASK in vitro, e.g., in an ELISA or a Western blot. Such double-stranded RNA complex or small molecule useful for detection may be provided with a label such as a fluorescent or radiolabel.

K. Articles of Manufacture and Kits

Another embodiment of the invention is an article of manufacture containing materials useful for the treatment of TASK expressing cancer. The article of manufacture comprises a container and a label or package insert on or associated with the container. Suitable containers include, for example, bottles, vials, syringes, etc. The containers may be formed from a variety of materials such as glass or plastic. The container holds a composition, which is effective for treating the cancer condition, and may have a sterile access port (for example the container may be an intravenous solution bag or a vial having a stopper pierceable by a hypodermic injection needle). At least one active agent in the composition is a double-stranded RNA complex or small molecule of the invention. The label or package insert indicates that the composition is used for treating cancer. The label or package insert will further comprise instructions for administering the double-stranded RNA complex or small molecule composition to the cancer patient. Additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for TASK-expressing cell killing assays, for purification or immunoprecipitation of TASK polypeptide from cells. Kits can be provided which contain the double-stranded RNA complex or small molecules for detection and quantitation of TASK polypeptide in vitro. As with the article of manufacture, the kit comprises a container and a label or package insert on or associated with the container. The container holds a composition comprising at least one double-stranded RNA complex or small molecule of the invention. Additional containers may be included that contain, e.g., diluents and buffers, control antibodies. The label or package insert may provide a description of the composition as well as instructions for the intended in vitro or diagnostic use.

L. Uses for TASK Polypeptides and TASK-Polypeptide Encoding Nucleic Acids

Nucleotide sequences (or their complement) encoding TASK polypeptides have various applications in the art of molecular biology, including uses as hybridization probes, in chromosome and gene mapping and in the generation of double-stranded RNA complex and DNA probes. TASK-encoding nucleic acid will also be useful for the preparation of TASK polypeptides by the recombinant techniques described herein.

The full-length native sequence TASK gene, or portions thereof, may be used as hybridization probes for a cDNA library to isolate the full-length TASK cDNA or to isolate still other cDNAs (for instance, those encoding naturally-occurring variants of TASK or TASK from other species) which have a desired sequence identity to the native TASK sequence disclosed herein. Optionally, the length of the probes will be about 20 to about 50 bases. The hybridization probes may be derived from at least partially novel regions of the full length native nucleotide sequence wherein those regions may be determined without undue experimentation or from genomic sequences including promoters, enhancer elements and introns of native sequence TASK. By way of example, a screening method will comprise isolating the coding region of the TASK gene using the known DNA sequence to synthesize a selected probe of about 40 bases. Hybridization probes may be labeled by a variety of labels, including radionucleotides such as ³²P or ³⁵S, or enzymatic labels such as alkaline phosphatase coupled to the probe via avidin/biotin coupling systems. Labeled probes having a sequence complementary to that of the TASK gene of the present invention can be used to screen libraries of human cDNA, genomic DNA or mRNA to determine which members of such libraries the probe hybridizes to. Hybridization techniques are described in further detail in the Examples below. Any EST sequences disclosed in the present application may similarly be employed as probes, using the methods disclosed herein.

Other useful fragments of the TASK-encoding nucleic acids include antisense or sense oligonucleotides comprising a singe-stranded nucleic acid sequence (either RNA or DNA) capable of binding to target TASK mRNA (sense) or TASK DNA (antisense) sequences. Antisense or sense oligonucleotides, according to the present invention, comprise a fragment of the coding region of TASK DNA. Such a fragment generally comprises at least about 14 nucleotides, preferably from about 14 to 30 nucleotides. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988).

Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. Such methods are encompassed by the present invention. The antisense oligonucleotides thus may be used to block expression of TASK proteins, wherein those TASK proteins may play a role in the induction of cancer in mammals. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Other examples of sense or antisense oligonucleotides include those oligonucleotides which are covalently linked to organic moieties, such as those described in WO 90/10048, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid sequence by any gene transfer method, including, for example, CaPO₄-mediated DNA transfection, electroporation, or by using gene transfer vectors such as Epstein-Barr virus. In a preferred procedure, an antisense or sense oligonucleotide is inserted into a suitable retroviral vector. A cell containing the target nucleic acid sequence is contacted with the recombinant retroviral vector, either in vivo or ex vivo. Suitable retroviral vectors include, but are not limited to, those derived from the murine retrovirus M-MuLV, N2 (a retrovirus derived from M-MuLV), or the double copy vectors designated DCT5A, DCT5B and DCT5C (see WO 90/13641).

Sense or antisense oligonucleotides also may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide may be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

Antisense or sense RNA or DNA molecules are generally at least about 5 nucleotides in length, alternatively at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length.

The probes may also be employed in PCR techniques to generate a pool of sequences for identification of closely related TASK coding sequences.

Nucleotide sequences encoding a TASK can also be used to construct hybridization probes for mapping the gene that encodes that TASK and for the genetic analysis of individuals with genetic disorders. The nucleotide sequences provided herein may be mapped to a chromosome and specific regions of a chromosome using known techniques, such as in situ hybridization, linkage analysis against known chromosomal markers, and hybridization screening with libraries.

When the coding sequences for TASK encode a protein which binds to another protein (example, where the TASK is a receptor), the TASK can be used in assays to identify the other proteins or molecules involved in the binding interaction. By such methods, inhibitors of the receptor/ligand binding interaction can be identified. Proteins involved in such binding interactions can also be used to screen for peptide or small molecule inhibitors or agonists of the binding interaction. Also, the receptor TASK can be used to isolate correlative ligand(s). Screening assays can be designed to find lead compounds that mimic the biological activity of a native TASK or a receptor for TASK. Such screening assays will include assays amenable to high-throughput screening of chemical libraries, making them particularly suitable for identifying small molecule drug candidates. Small molecules contemplated include synthetic organic or inorganic compounds. The assays can be performed in a variety of formats, including protein-protein binding assays, biochemical screening assays, immunoassays and cell based assays, which are well characterized in the art. In addition, screening methods can be used where a double-stranded RNA complex suppresses the expression or activity of a TASK target gene, and this can be used to measure the efficacy of other TASK antagonists by comparing the reduction in TASK activity or expression of the double-stranded RNA complex with the reduction in activity or expression of a TASK antagonist.

Nucleic acids which encode TASK or its modified forms can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. In one embodiment, cDNA encoding TASK can be used to clone genomic DNA encoding TASK in accordance with established techniques and the genomic sequences used to generate transgenic animals that contain cells which express DNA encoding TASK. Methods for generating transgenic animals, particularly animals such as mice or rats, have become conventional in the art and are described, for example, in U.S. Pat. Nos. 4,736,866 and 4,870,009. Typically, particular cells would be targeted for TASK transgene incorporation with tissue-specific enhancers. Transgenic animals that include a copy of a transgene encoding TASK introduced into the germ line of the animal at an embryonic stage can be used to examine the effect of increased expression of DNA encoding TASK. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologues of TASK can be used to construct a TASK “knock out” animal which has a defective or altered gene encoding TASK as a result of homologous recombination between the endogenous gene encoding TASK and altered genomic DNA encoding TASK introduced into an embryonic stem cell of the animal. For example, cDNA encoding TASK can be used to clone genomic DNA encoding TASK in accordance with established techniques. A portion of the genomic DNA encoding TASK can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the TASK polypeptide. In circumstances where ablation of the TASK gene through knockout proves embryonic lethal to the animal and few/no progeny are produced, then the inducible pHUSH vector system can be used to create a transgenic mouse that will produce double-stranded RNA complex upon induction, and the consequences of reduction of gene expression can be determined in the adult animal.

Nucleic acid encoding the TASK polypeptides may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83:4143-4146 [1986]). The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11, 205-210 [1993]). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). For review of gene marking and gene therapy protocols see Anderson et al., Science 256, 808-813 (1992).

The nucleic acid molecules encoding the TASK polypeptides or fragments thereof described herein are useful for chromosome identification. In this regard, there exists an ongoing need to identify new chromosome markers, since relatively few chromosome-marking reagents, based upon actual sequence data are presently available. Each TASK nucleic acid molecule of the present invention can be used as a chromosome marker.

The TASK polypeptides and nucleic acid molecules of the present invention may also be used diagnostically for tissue typing, wherein the TASK polypeptides of the present invention may be differentially expressed in one tissue as compared to another, preferably in a diseased tissue as compared to a normal tissue of the same tissue type. TASK nucleic acid molecules will find use for generating probes for PCR, Northern analysis, Southern analysis and Western analysis.

This invention encompasses methods of screening compounds to identify those that mimic the TASK polypeptide (agonists) or prevent the effect of the TASK polypeptide (antagonists). Screening assays for antagonist drug candidates are designed to identify compounds that bind or complex with the TASK polypeptides encoded by the genes identified herein, or otherwise interfere with the interaction of the encoded polypeptides with other cellular proteins, including e.g., inhibiting the expression of TASK polypeptide from cells. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

All assays for antagonists are common in that they call for contacting the drug candidate with a TASK polypeptide encoded by a nucleic acid identified herein under conditions and for a time sufficient to allow these two components to interact.

In binding assays, the interaction is binding and the complex formed can be isolated or detected in the reaction mixture. In a particular embodiment, the TASK polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the TASK polypeptide and drying. Alternatively, an immobilized antibody, e.g., a monoclonal antibody, specific for the TASK polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular TASK polypeptide encoded by a gene identified herein, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GALA, and another, in which candidate activating proteins are fused to the activation domain. The expression of a GAL1-lacZ reporter gene under control of a GAL4-activated promoter depends on reconstitution of GALA activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of a gene encoding a TASK polypeptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the TASK polypeptide may be added to a cell along with the compound to be screened for a particular activity and the ability of the compound to inhibit the activity of interest in the presence of the TASK polypeptide indicates that the compound is an antagonist to the TASK polypeptide. Alternatively, antagonists may be detected by combining the TASK polypeptide and a potential antagonist with membrane-bound TASK polypeptide receptors or recombinant receptors under appropriate conditions for a competitive inhibition assay. The TASK polypeptide can be labeled, such as by radioactivity, such that the number of TASK polypeptide molecules bound can be used to determine the effectiveness of the potential antagonist. Preferably, expression cloning is employed wherein polyadenylated RNA is prepared from a cell responsive to the TASK polypeptide and a cDNA library created from this RNA is divided into pools and used to transfect COS cells or other cells that are not responsive to the TASK polypeptide. Transfected cells that are grown on glass slides are exposed to labeled TASK polypeptide. The TASK polypeptide can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach for binding identification, labeled TASK polypeptide can be photoaffinity-linked with cell membrane or extract preparations that express the receptor molecule. Cross-linked material is resolved by PAGE and exposed to X-ray film. The labeled complex containing the bound proteins can be excised, resolved into peptide fragments, and subjected to protein micro-sequencing. The amino acid sequence obtained from micro-sequencing would be used to design a set of degenerate oligonucleotide probes to screen a cDNA library to identify the gene encoding the putative binding partner.

In another assay for antagonists, mammalian cells or a membrane preparation expressing the receptor would be incubated with labeled TASK polypeptide in the presence of the candidate compound. The ability of the compound to enhance or block this interaction could then be measured.

More specific examples of potential antagonists include an oligonucleotide that binds to the fusions of immunoglobulin with TASK polypeptide, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential antagonist may be a closely related protein, for example, a mutated form of the TASK polypeptide that recognizes the receptor but imparts no effect, thereby competitively inhibiting the action of the TASK polypeptide.

Another potential TASK polypeptide antagonist is an antisense RNA or DNA construct prepared using antisense technology, where, e.g., an antisense RNA or DNA molecule acts to block directly the translation of mRNA by hybridizing to targeted mRNA and preventing protein translation. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature TASK polypeptides herein, is used to design an antisense RNA oligonucleotide of from about 10 to 40 base pairs in length. A DNA oligonucleotide is designed to be complementary to a region of the gene involved in transcription (triple helix—see Lee et al., Nucl. Acids Res., 6:3073 (1979); Cooney et al., Science, 241: 456 (1988); Dervan et al., Science, 251:1360 (1991)), thereby preventing transcription and the production of the TASK polypeptide. The antisense RNA oligonucleotide hybridizes to the mRNA in vivo and blocks translation of the mRNA molecule into the TASK polypeptide (antisense—Okano, Neurochem., 56:560 (1991); Oligodeoxynucleotides as Antisense Inhibitors of Gene Expression (CRC Press: Boca Raton, Fla., 1988). The oligonucleotides described above can also be delivered to cells such that the antisense RNA or DNA may be expressed in vivo to inhibit production of the TASK polypeptide. When antisense DNA is used, oligodeoxyribonucleotides derived from the translation-initiation site, e.g., between about −10 and +10 positions of the target gene nucleotide sequence, are preferred.

Potential antagonists include small molecules that bind to the active site, or other relevant binding site of the TASK polypeptide, thereby blocking the normal biological activity of the TASK polypeptide. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology, 4:469-471 (1994), and PCT publication No. WO 97/33551 (published Sep. 18, 1997).

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra.

These small molecules can be identified by any one or more of the screening assays discussed hereinabove and/or by any other screening techniques well known for those skilled in the art.

Isolated TASK polypeptide-encoding nucleic acid can be used herein for recombinantly producing TASK polypeptide using techniques well known in the art and as described herein. In turn, the produced TASK polypeptides can be employed for generating anti-TASK antibodies using techniques well known in the art and as described herein.

Antibodies specifically binding a TASK polypeptide identified herein, as well as other molecules identified by the screening assays disclosed hereinbefore, can be administered for the treatment of various disorders, including cancer, in the form of pharmaceutical compositions.

Internalizing antibodies are preferred as the TASK polypeptide is intracellular. However, lipofections or liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993).

The formulation herein may also contain more than one active compound as necessary for the particular indication being treated, preferably those with complementary activities that do not adversely affect each other. Alternatively, or in addition, the composition may comprise an agent that enhances its function, such as, for example, a cytotoxic agent, cytokine, chemotherapeutic agent, or growth-inhibitory agent. Such molecules are suitably present in combination in amounts that are effective for the purpose intended.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### Example 1

### Inducible Vector for Expression of RNA Coding Regions

To generate a vector suitable for tetracycline-inducible expression of RNA coding regions, we incorporated a modified polIII promoter (a H1 promoter comprising a one or more tetR operon (Tet-O) sites) into a single retroviral expression plasmid to direct conditional expression of RNA coding regions in cells of choice. In the off state, the Tet repressor protein (TetR) binds the modified polIII promoter, thereby preventing siRNA expression. However, in the presence of a tetracycline analog, doxycycline (Dox), the Tet repressor protein is released from the promoter, permitting siRNA transcription. Using retroviral delivery, followed by selection for puromycin resistance, cell clones with stable integration of this RNA coding region-expression cassette can be rapidly generated.

In one embodiment, our vector system is comprised of a kanamycin-resistant, H1 promoter-driven shRNA expression shuttle plasmid and an ampicillin-resistant retroviral vector backbone that contains a codon-optimized TetR-IRES-puromycin cassette to enable Tet-regulated shRNA expression. Knockdown vectors are constructed by cloning shRNA-encoding oligonucleotides into the shuttle vector followed by a Gateway recombination reaction (Invitrogen, USA) to transfer the shRNA cassette in the retroviral vector. The shRNA-encoding oligonucleotide is synthesized to include a termination sequence (5-′TTTTTT-3′ SEQ ID NO:______). All constructs were verified by sequencing. Gene knockdown using these shRNAs was first verified in transient assays. Reduction of gene expression is achieved by the steps of designing and cloning shRNA sequences into the shRNA expression shuttle plasmid, transferring the shRNA-H1 cassette from the shuttle plasmid into the retroviral vector backbone by recombination, and packaging the completed vector into a retroviral particle.

shRNA hairpin shuttle vectors were constructed by PCR sub-cloning the pSuperior-H1 promoter (OligoEngine, Seattle, Wash.), adding XbaI, SpeI, and AgeI sites, and then TOPO-cloning into pENTR/D (Invitrogen) to form pShuttle-H1-shRNA. A similar strategy was employed to add a second TetO₂ operon to form pShuttle-H1-shRNA-2x-TetO₂ by ligating dsDNA into the MslI and HindIII sites of the original vector (described below). A diagram of pSHUTTLE is shown in FIG. 1.

A diagram of a retroviral vector backbone, termed pHUSH-GW, is shown in FIG. 1. Briefly, pHUSH-GW was constructed by subcloning a human β-actin TetR cassette into the BglII and EcoRI sites of pQCXIP, a Moloney murine leukemia virus (MMLV) retroviral expression vector (Clontech, USA). This vector was then converted into a Gateway (Invitrogen, Carlsbad Calif.) destination vector to facilitate the rapid introduction of hairpins into the system by recombination-based cloning. Briefly, a Gateway acceptor module was ligated to a blunted MfeI site to form pHUSH-GW, and recombination reactions of pShuttle-H1 and pHUSH-GW were performed according to the manufacturer's instructions.

### Example 2

### Optimization of the Inducible RNA Coding Region Expression Vector

Performance of the inducible vector was optimized by codon optimizing the TetR sequence for mammalian expression. The performance of the vector was further optimized by incorporating a modified polIII promoter comprising two TetO binding sites. These elements were included in several of the retroviral vectors described in the remainder of the Examples, except as otherwise indicated below.

Codon Optimized TetR Significantly Increased Repression of shRNA Expression

The TetR coding region was codon optimized for mammalian expression using the DNAWorks program (available from the NIH, Bethesda Md.). The polynucleotide and amino acid sequences of the codon-optimized TetR is shown in SEQ ID NO:1 and SEQ ID NO:2, respectively. Using a compatible end derived from a BsaI site, we ligated the codon optimized TetR in-frame to the NcoI start site of pDRIVE-5′RU (Invitrogen, San Diego, Calif.). The human β-actin TetR cassette was then PCR amplified using Pfu Turbo Polymerase (Stratagene, La Jolla, Calif.), adding a four restriction site ‘polylinker’ (MfeI, ClaI, SpeI, AgeI) upstream of the promoter, and was subcloned into pIRES-Puro2 (Clontech, Palo Alto, Calif.) using MfeI and EcoRI sites to form EV-pHUSH. The 5′ UTR of the human β-actin cassette harbored an unwanted splicing donor that may form an intron with the ‘IVS’ (intervening sequence) sequence derived from pIRES-Puro2, so the IVS sequence was removed by swapping the original IVS-IRES-Puro module with a similar IRES-Puro fragment derived from PQCXIP (Clontech, Palo Alto, Calif.).

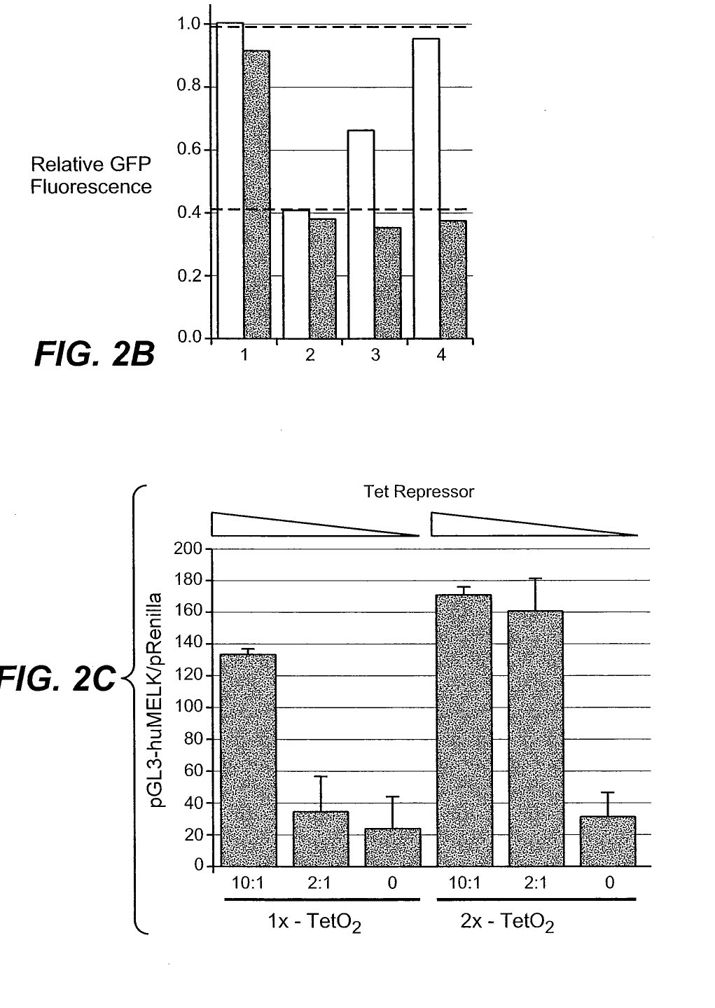

To confirm that codon optimization of the TetR coding region increased expression of the TetR protein, Western analysis was performed. Briefly, plasmids containing the retroviral backbone comprising either wild type TetR (WT) or the codon optimized TetR (OPT) were transiently transfected into 293T cells using lipofectin (commercially available from GIBCO-BRL, Gaithersburg, Md.). Cell lysates were prepared by addition of 50 mM HEPES pH 7.4, 100 mM NaCl, 50 mM NaF, 5 mM B-glycerophosphate, 2 mM EGTA, 1 mM Na vanadate, 1% TX100 to a cell pellet, resolved on a 4-12% acrylamide gel and transferred to a PVDF membrane. Membranes were sequentially probed with Mouse α-TetR (1:5000; MolBiTec) and Rabbit α-Tubulin-HRP (1:5000, Santa Cruz Biotech, Santa Cruz, Calif.). The results of this experiment are shown in FIG. 2A. Codon optimization of the TetR coding region (shown in lane “Opt”) significantly increased expression of the TetR protein as compared to expression of TetR protein from the wildtype coding sequence (shown in lane “Wt”). “Ev” signifies negative control using retroviral vector lacking a shRNA cassette.

To confirm that maximizing TetR expression prevented unwanted silencing of target genes in the absence of Dox, cells (293HEK) stably expressing a GFP-MELK fusion construct were transfected with the following pHUSH vector series containing a Melk targeting shRNA: pHUSH lacking an H1-shRNA cassette (empty vector, lane 1 of FIG. 2B); pHUSH (lane 2 of FIG. 2B); pHUSH^{-IVS} (removal of the synthetic intron sequence between the TetR ORF and the IRES, lane 3 of FIG. 2B); pHUSH^{-IVS}TetR^{Opt} (replacement of the TetR in pHUSH^{-IVS} with a codon-optimized TetR, lane 4 of FIG. 2B). Stable pools of cells were selected by culturing in the presence of 3 μg/ml puromycin. The resulting stable pools were treated in the absence (open bars) or presence (filled bars) of 1 μg/1 ml doxycycline for five days and the level of GFP-Melk expression analyzed by FACS. The effectiveness of each construct to repress shRNA expression was assessed by comparing the level of eGFP-Melk fluorescence in the absence of Dox, to the level of eGFP-Melk fluorescence in cells containing a control plasmid comprising the pHUSH retroviral backbone but lacking a shRNA cassette (pHUSH-EV, lane 1). Data was normalized to the mean fluorescence intensity of cells containing the pHUSH empty vector control (lane 1 of FIG. 2B).

The results of this experiment are shown in FIG. 2B. Removal of the IVS and codon optimization of the TetR coding region significantly increased the repression of shRNA expression. As shown in FIG. 2B, in the absence of Dox (open bars), only the pHUSH^{-IVS}TetR^{opt} pool exhibited full repression of shRNA expression (lane 4). In contrast, partial repression (about 70% of maximal eGFP-Melk expression) was obtained using pHUSH^{-IVS} (removal of the IVS but containing the wildtype TetR coding region, lane 3), while the original pHUSH vector displayed a complete lack of shRNA repression, as evident by maximal GFP-Melk silencing in the presence or absence of Dox (lane 2). As expected, all three siRNA-containing vectors generated similar levels of eGFP-Melk expression in the presence of Dox (filled bars).

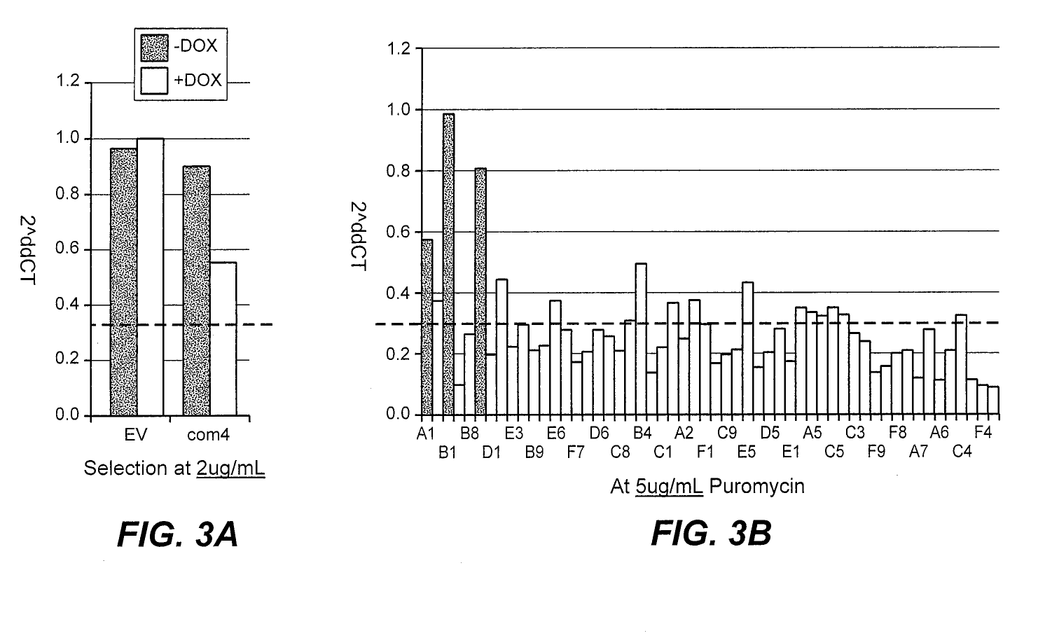

To determine whether expression strength of the TetR coding region correlated with the level of shRNA produced, LOX-1 cells were transfected with an empty pHUSH vector (EV) or pHUSH comprising a cassette comprising a TASK121 (B-Raf)-directed shRNA (mutA or com4). Cells were placed under 2 ug/ml (EV, black bar and com4 or mutA, open bars) or 5 μg/ml puromycin (gray bars) selection for two weeks. The resulting stable pool (A in FIG. 3) or multiple stable clones (B in FIG. 3) were cultured in the absence (open bars in FIG. 3) or presence (filled bars in FIG. 3) or 1 ug/ml Dox for 3 days. The level of b-Raf knockdown was determined by quantitative RT-PCR (Taqman™) and the data normalized by the 2̂ddCT method where “1” represents the level of b-Raf in control cells. The results of this experiment are shown in FIG. 3. Expression strength of the TetR coding region correlated with the level of shRNA produced. Dotted lines represent the average knockdown (43% knockdown in A, and 73% knock down in B).

Presence of Multiple TetO2 Operons within the Pol III Promoter Enhanced Regulation of TetR Repression

To determine whether the placement of multiple TetR operons within a PolIII promoter resulted in increased regulation of TetR repression, we generated a shRNA expression vector comprising two Tet operons (TetO) within the H1 promoter of the pHUSH vector.

We modified the original pShuttle-H1-shRNA plasmid containing a single TetO₂ site (positioned between the TATA box of the promoter and the transcriptional start-site) by adding an additional TetO₂ operon upstream of the TATA box, resulting in the 2x-TetO2-H1 promoter. The final promoter contained TetO2 operons positioned (1) between the TATA box and the transcriptional start site, and (2) upstream of the TATA box. Shuttle vector pShuttle-H1-TetO-2x was generated by annealing and ligating the following oligos to the MslI and HindIII sites in pShuttle-H1:

sense

(SEQ ID NO: 7)

5′ACGTGAAATCCCTATCAGTGATAGAGACTTATAAGTTCCCTATCAGTG
ATAGAGATCTAAAGGGAAAA 3′
Anti-sense

(SEQ ID NO: 8)

5′AGCTTTTTCCCTTTAGATCTCTATCACTGATAGGGAACTTATAAGTCT
CTATCACTGATAGGGATTTCACGT 3″
(tetO in bold/underlined

The polynucleotide sequence of the 2x-TetO2-H1 promoter comprised the following sequence:

(SEQ ID NO: 19)

CGAACGCTGACGTCATCAACCCGCTCCAAGGAATCGCGGGCCCAGTGTCA
CTAGGCGGGAACACCCAGCGCGCGTGCGCCCTGGCAGGAAGATGGCTGTG
AGGGACAGGGGAGTGGCGCCCTGCAATATTTGCATGTCGCTATGTGTTCT
GGGAAATCACCATAAACGTGAAATCCCTATCAGTGATAGAGACTTATAAG
TTCCCTATCAGTGATAGAGATCCCC.

A Melk directed shRNA was ligated into both 1x-TetO₂ and 2x-TetO₂ pShuttle-H1 vectors. A GL3-Melk reporter construct was generated by first ligating to the XbaI site of pGL3-Luc (Promega, Madison, Wis.) a multiple cloning site downstream of the luciferase stop codon to form pGL3-Luc-MCS. The pGL3-MELK vector was then constructed by ligating the entire cDNA of MELK into pGL3-Luc-MCS, resulting in a luciferase-Melk transcript-fusion (nucleic acid sequence: SEQ ID NO5; amino acid sequence: SEQ ID NO:6) in which only Luciferase is translated. Using pGL3-Melk as a reporter, the 1x- and 2x-TetO₂-H1 promoters were co-transfected with increasing amounts of a CMV-TetR^{Opt} expression plasmid in the absence of Dox. For both shRNA promoter variants, maximal repression was achieved when the TetR expression construct was supplied in molar excess (TetR:H1-shRNA; 10:1) while >80% knockdown was achieved in the absence (“0” in FIG. 2C) of TetR (FIG. 2C). When the TetR expression plasmid was limited to a 2:1 molar ratio relative to the H1-shRNA plasmid, nearly maximal shRNA expression was observed with the 1x-TetO₂ construct. In contrast, shRNA repression was maintained with the 2x-TetO₂ construct. These results suggest that the presence of multiple TetO2 operons within a polIII promoter enhanced regulated shRNA expression. In particular, the use of two TetR operons enhanced the ability to regulate shRNA expression when TetR expression is limiting.

### Example 3

### Dose-Response of Dox-Induced Gene Knock-Down In Vitro and In Vivo

To determine whether Dox dose correlated with the level of inhibition of target gene expression, we performed dose-response experiments in which the level of Dox was varied, using in vitro and in vivo models.

First, we examined the regulated shRNA-mediated depletion of luciferase gene expression using luciferase-expressing SVT2 cells in vitro. Briefly, cells were infected with a pHUSH inducible retroviral vector comprising the following shRNA targeting the luciferase coding region:

shLUC-GL3

(SEQ ID NO: 9)

5′-GAT CCC CCT TAC GCT GAG TAC TTC GAT TCA AGA GAT
CGA AGT ACT CAG CGT AAG TTT TTT GGA AA-3′.

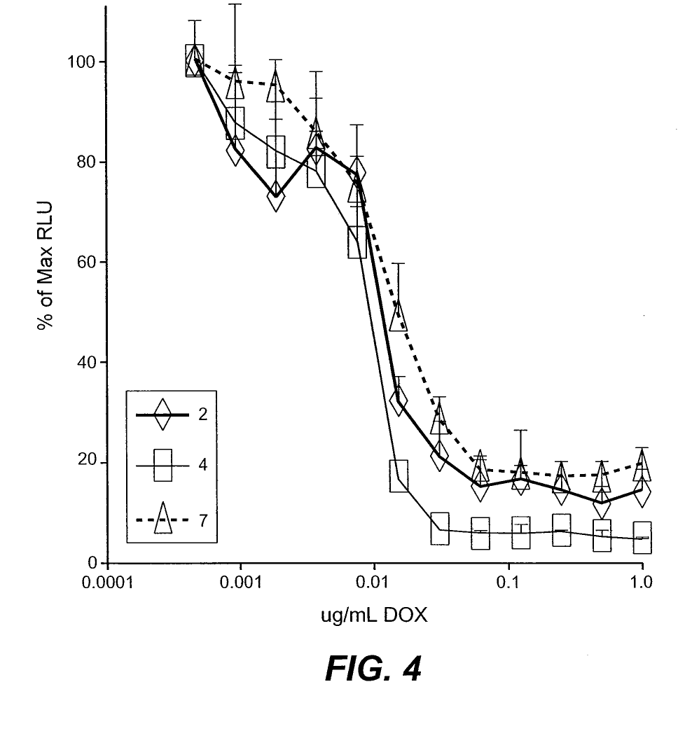

Cells were selected with puromycin and individual clones with Dox regulated luciferase expression identified (referred to as SVT2_GL3_pHUSH shLUC). Cells were cultured for 2 days (diamonds), 4 days (squares) or 7 days (triangles) at the indicated dose of doxycycline (see FIG. 4) and then assayed for luciferase activity using standard methods.

The results of this experiment are shown in FIG. 4. Dox dose correlated with level of inhibition of target gene expression. A dose of 0.01 μg/ml and 0.1 μg/ml of doxycycline was optimum for the suppression of the luciferase activity in this experiment. Moreover, the amount of doxycycline could be titrated as appropriate to generate a complete knockdown or an intermediate knockdown of gene expression.

Next, we examined the regulated shRNA-mediated inhibition of luciferase gene expression using luciferase-expressing SVT2 cells in an in vivo tumor growth assay. A SVT2-GL3˜pHUSH_shLUC clone, generated as described above, was injected subcutaneously in the right flank using standard methods. Ten days post injection, animals were administered sucrose water alone (animals 1A and 1B), sucrose with doxycycline at 1 mg/ml (animals 2A and 2B), 0.1 mg/ml (animals 3A and 3B) or 0.01 mg/ml (animals 4A and 4B). Mice were injected with 250 μL 200 mg/kg D-luciferin (Biotium, Hayward, Calif.) and anesthetized using isoflurane. Bioluminescence images were acquired using a cooled intensified charge-coupled device camera in an in vivo imaging system (Xenogen, Alameda, Calif.). Image acquisition times were about 1 minute. Images were processed by co-registering a reference image with a thresholded bioluminescence data image.

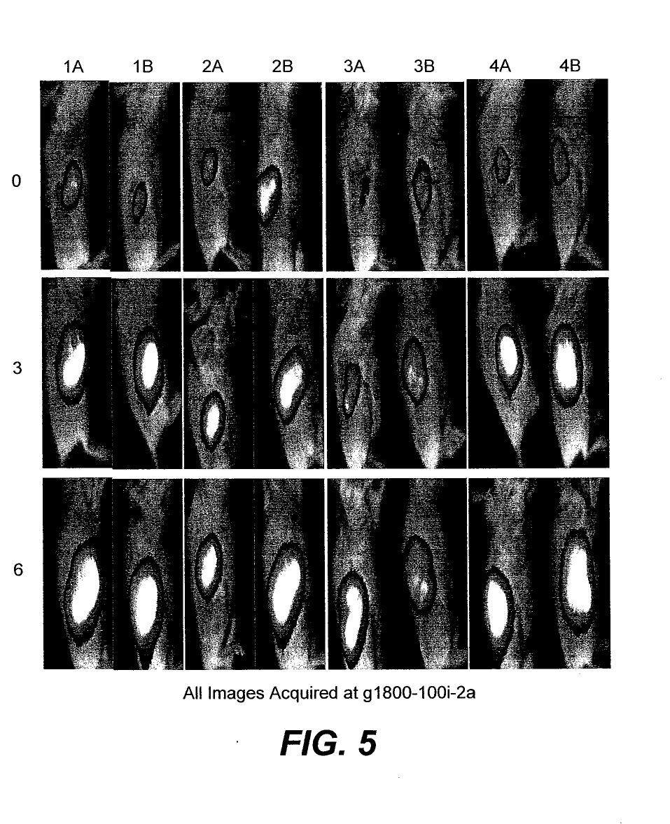

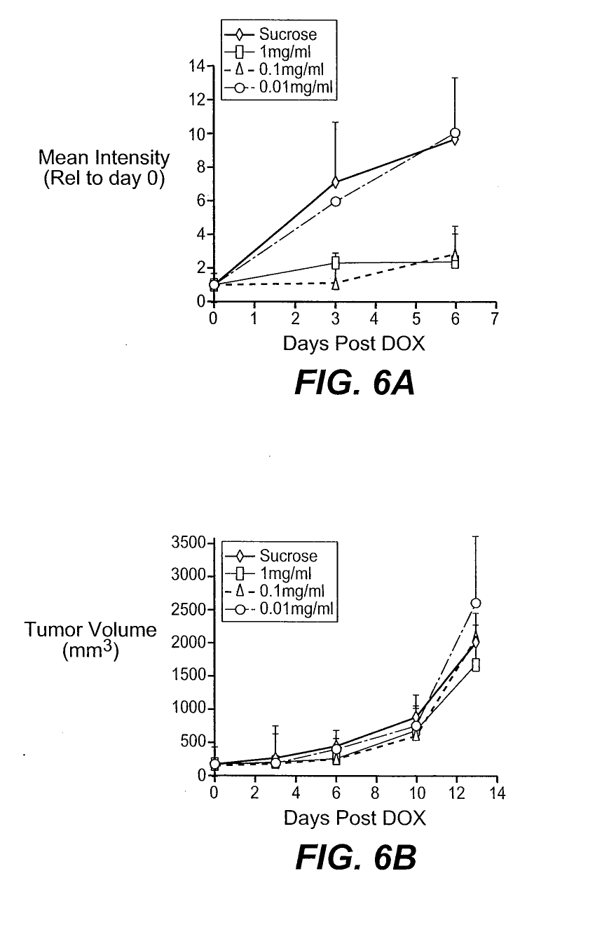

The results of this experiment are shown in FIGS. 5 and 6. FIG. 5 shows representative bioluminescence images of two animals within each treatment arm at time points 0, 3 and 6 days after addition of doxycycline to the drinking water. Treatment with 1.0 mg/ml Dox resulted in reduced luciferase activity compared to control animals, and treatment with 0.1 mg/ml Dox resulted in almost complete reduction of luciferase activity compared with control animals. By contrast, treatment with 0.01 mg/ml Dox resulted in luciferase activity that is comparable to that of control animals. FIG. 6A shows the relative change in mean bioluminescence at each timepoint and treatment condition (n=5 per doxycycline).

To determine whether Dox or shRNA expression influenced tumor growth in vivo, tumor volume measurements were recorded for the tumors at each concentration of Dox. The results of this experiment, shown in FIG. 6B, show that Dox or shRNA treatment per se did not influence tumor growth in vivo.

### Example 4

### Use of an Inducible siRNA Expression Vector for Delivery of RNA Coding Regions in Mouse Embryonic Stem Cells and for the Creation of Transgenic Mice

A. We generated an inducible expression vector for delivery of RNA coding regions in mouse embryonic stem cells and for the creation of transgenic mice as follows:

The following shRNA directed toward the murine Melk kinase gene was inserted into pShuttle-H1-TetO₂1X (termed pShuttle-H1 in the remainder of this example):

shMELK-B

(SEQ ID NO: 10)

5′-GAT CCC GAG ATT AGT GGA AGA TAT CTT CAA GAG AGA
TAT CTT CCA CTA ATC TCT TTT TTG GAA A-3′.

The H1-shRNA cassette was subcloned into vector backbone that was modified to (a) delete retroviral backbone sequences and (b) transfer remaining sequences into a plasmid. Briefly, the human-b-actin promoter driving the TetR was subcloned into pIRES-Puro2 to replace the original CMV promoter. A modified H1 promoter 40 containing a single TetR operon with a Melk-targeting shRNA cassette was inserted upstream to the human-b-actin promoter. Using this plasmid as the base vector, we generated two additional versions for analysis: pHUSH-IVS in which a synthetic intron sequence between the TetR ORF and the IRES was removed, and pHUSH-IVSTetROpt, in which the original TetR sequence within pHUSH-IVS was replaced by a TetR expression cassette that was codon-optimized for expression in mammalian cells. pHUSH-IVSTetROpt represents the non-viral vector employed in murine ES cell studies and is based on the pIRES-Puro2 backbone, which lacks the 5′ hybrid CMV/LTR, the psi+ packaging signal, and the 3′ sinLTR found in the retroviral vector described herein. In addition, the XbaI-AgeI fragment from pShuttle-H1-shRNA was ligated into the compatible SpeI-Agel site of the modified plasmid backbone to form the complete vector, the structure of which is depicted in FIG. 7A.

ES cell culture, electroporation and cell line selection were carried out as described (Hogan et al., Manipulating the mouse embryo, 2^{nd} Ed., Cold Spring Harbor Press. 1994). ES cell culture medium was made of Dulbecco's modified Eagle medium (DMEM), supplemented with 15% heat-inactivated fetal calf serum, 2 mM L-glutamine, 0.1 mM non-essential amino acids, 1000 TU/ml recombinant human leukemia inhibitory factor (LIF; ESGRO), 0.1 mM b-mercaptoethanol, 50 U/ml penicillin, and 50 μg/ml streptomycin. To generate transgenic ES cell lines, 1×10⁷ of R1ES cells (Nagy et al., Proc. Natl. Acad. Sci. USA 90:8424-8428 (1993)) were electroporated with 20 μg of linearized retroviral vector DNA and selected in ES cell culture medium containing 1.0 μg/ml puromycin. Individual clones were picked, and those bearing a high level of TetR expression were assessed for Melk kinase gene knockdown after 3 days of Dox treatment. Melk kinase gene expression was analyzed using taqman analysis.

The results of this experiment are shown in FIG. 7B. 90-95% reduction in Melk kinase gene transcription was observed in each of three clones, as compared with untreated cells. These results demonstrated that the inducible vector effectively reduced target gene expression in mouse ES cells in a Dox dependent manner. FIG. 7A shows an inducible vector capable of delivering shRNA in a transgenic animal.

We also assessed the phenotypic effect of Melk kinase knockdown in embryoid body (EBs) development. EBs are a powerful tool to study gene function because they recapitulate early embryonic development in vitro. By day 7-10, EB differentiation is characterized by synchronized ectodermal, endodermal and the mesodermal germ layer development, the latter of which is specifically indicated by the appearance of spontaneously and rhythmically contracting cardiomyocytes. ES cell clones generated as described above and control parental cells (R1 in FIG. 7C) were subject to differentiation in media without and without 1.0 μg/ml doxycycline. ES cell differentiation medium was made of Dulbecco's modified Eagle medium (DMEM), supplemented with 10% heat-inactivated fetal calf serum, 50 U/ml penicillin, and 50 μg/nl streptomycin. For in vitro differentiation, ES cells were first cultured in ES cell medium in hanging drops (600 cells/30 μl/drop) for one day and then in differentiation medium in suspension as embryoid bodies in bacteriological petri dishes for 7 days. Embryoid bodies were then transferred to tissue culture plates coated with 0.1% gelatin for continuous culture in differentiation medium. Colonies with beating cardiomyocytes were scored at day 11, 13 and 15 after embryoid body formation. As compared to a Luciferase shRNA control (shLuc) or WT cells (R1), we observed a significant reduction in percent EB colonies positive for beating cardiomyocytes in the presence of Dox with shMELK-Q1-pHUSH^{IVS}TetR^{opt}2xTetO₂ (shMelk, FIG. 7C).

B. We assessed whether Melk expression knock-down was maintained in differentiated ES cells. ES cells and EBs were generated as described above using vectors bearing the shRNA described above and further bearing either the H1-TetO₂-1X or H1-TetO2-2X promoters. ES cell were differentiated into embryoid bodies in the presence or absence of 1 ug/ml of Dox, then the level of Melk expression was determined by qRT-PCR. Level of Melk expression was significantly

C. We generated transgenic mice comprising the inducible shRNA expression vector, and assessed whether transgenic progeny displayed regulatable expression of RNA coding regions. Transgenic ES cell lines generated as described in Example 4 (A), above, were injected into blastocysts of C57BL/6 mice to generate male chimeras with germ-line transmission, which were then bred to C57BL/6 females. Progeny from the breeding were genotyped by PCR amplification of TetR transgene. 15 F1 heterozygous progeny were positive for TetR expression. F1 heterozygous progeny were bred again to C57BL/6 females, and progeny from this breeding were genotyped by PCR amplification the TetR transgene, and 32 F2 heterozygous progeny were positive for TetR expression. Melk expression levels were assessed in F2 heterozygous progeny and wildtype animals following treatment in the presence or absence of 1 mg/ml Dox in drinking water for seven days. Tissues were harvested and expression of Melk mRNA was assessed by qPCR. Expression of Melk was repressed in large intestine, small intestine, T cells, and other tissues in heterozygous progeny treated with Dox.

### Example 5

### Insertion of a PolIII Expression Cassette into an Inducible Expression Vector Permitted Regulated Expression of Proteins

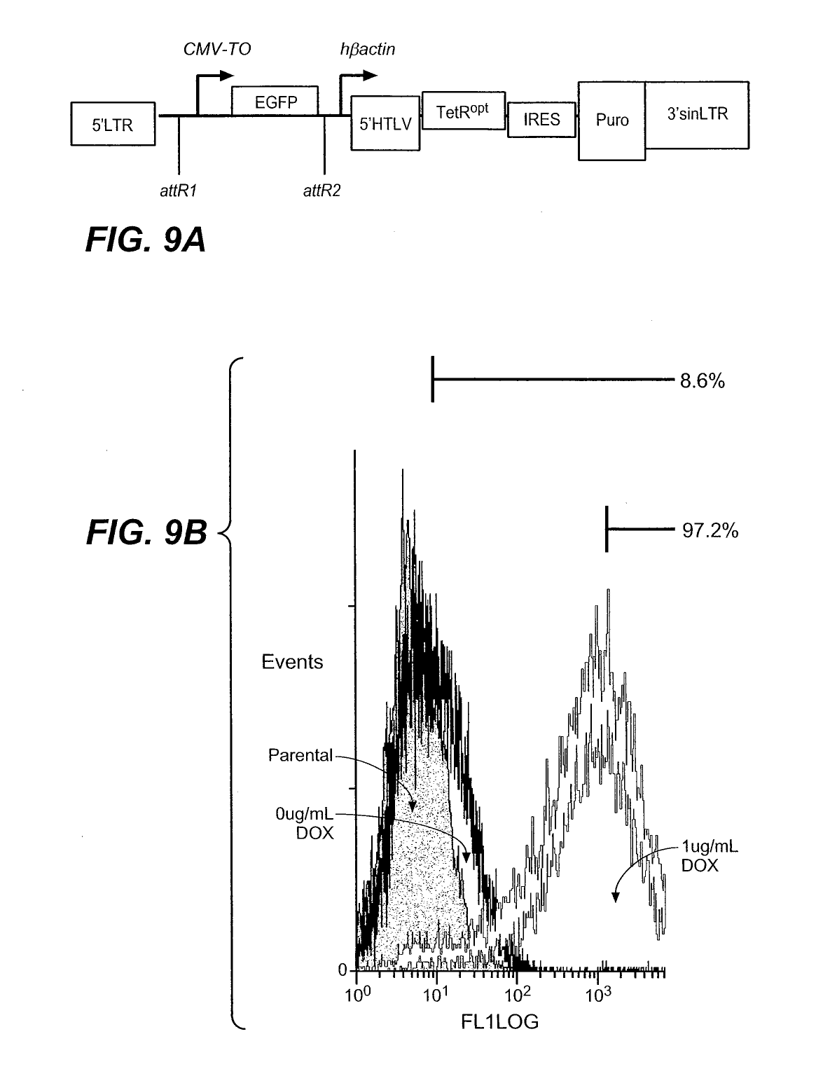

To obtain regulated expression of proteins, a shuttle vector comprising a polII promoter (a modified CMV promoter in combination with the Tet operator (CMV-TO in FIG. 9A)) driving expression of the GFP gene was generated. Briefly, the proEX shuttle plasmid, termed pShuttle-CMV-TO, was constructed by PCR amplifying pcDNA4/TO (Invitrogen), and TOPO-cloning the PCR amplicon into pENTR/D as above. To generate pShuttle-CMV-TO-EGFP, an EGFP fragment was generated by cutting pEGFP-C1 (Clontech, Mountain View, Calif.) with AgeI (subsequently blunted with T4 polymerase) and MfeI, and this fragment was ligated to the PmeI and EcoRI sites present within pShuttle-CMV-TO. Recombination of the shuttle vector into the retroviral vector backbone described in Example 1 yielded vector proEX, which is depicted in FIG. 9A.

To demonstrate that GFP expression from pHUSH-proEX was regulated yet robust, HCT116 cells (ATCC accession number CCL-247) were transduced with a pHUSH-proEX virus, selected with puromycin, the cells incubated in the presence or absence of 1 μg/ml doxycycline for 48 hours and the amount of GFP produced was assessed by FACS analysis. The results of this experiment, shown in FIG. 9B, demonstrate that expression of GFP was inducible and robust.

### Example 6

### Modulating BRAF/TASK 121 Expression Using shRNA

The following reagents were used in this study:

TASK121 shRNA-1 (sense)

(SEQ ID NO: 11)

5′-GAT CCC CAG AAT TGG ATC TGG ATC ATT TCA AGA GAA
TGA TCC AGA TCC AAT TCT TTT TTT GGA AA-3′
TASK121 shRNA-1 (antisense)

(SEQ ID NO: 12)

5′-AGC TTT TCC AAA AAA AGA ATT GGA TCT GGA TCA TTC
TCT TGA AAT GAT CCA GAT CCA ATT CTG GG-3′
TASK121 shRNA-2 (sense)

(SEQ ID NO: 13)

5′-GAT CCC CGC TAC AGA GAA ATC TCG ATT TCA AGA GAA
TCG AGA TTT CTC TGT AGC TTT TTT GGA AA-3′
TASK121 shRNA-2 (antisense)

(SEQ ID NO: 14)

5′-AGC TTT TCC AAA AAA GCT ACA GAG AAA TCT CGA TTC
TCT TGA AAT CGA GAT TTC TCT GTA GCG GG-3′
Luciferase shRNA (sense)

(SEQ ID NO: 15)

5′-GAT CCC CCT TAC GCT GAG TAC TTC GAT TCA AGA GAT
CGA AGT ACT CAG CGT AAG TTT TTT GGA AA-3′
Luciferase shRNA (antisense)

(SEQ ID NO: 16)

5′-AGC TTT TCC AAA AAA CTT ACG CTG AGT ACT TCG ATC
TCT TGA ATC GAA GTA CTC AGC GTA AGG GG-3′
EGFP shRNA (sense)

(SEQ ID NO: 17)

5′-GAT CCC CAG ATC CGC CAC AAC ATC GAT TCA AGA GAT
CGA TGT TGT GGC GGA TCT TGT TTT TTG GAA A-3′
EGFP shRNA (antisense)

(SEQ ID NO: 18)

5′-AGC TTT TCC AAA AAA CAA GAT CCG CCA CAA CAT CGA
TCT CTT GAA TCG ATG TTG TGG CGG ATC TGG G-3′

The complementary double-stranded oligonucleotides were inserted into the pHUSH vector using BglII and HindIII restriction enzyme sites. The pHUSH system used in this example was comprised of a kanamycin-resistant, H1 promoter-driven shRNA expression shuttle plasmid and an ampicillin-resistant retroviral vector that contained a codon-optimized TetR-IRES-puromycin cassette to enable Tet-regulated shRNA expression. Knockdown vectors were constructed by cloning shRNA oligos into the shuttle vector followed by a Gateway recombination reaction (Invitrogen, Carlsbad Calif.) to transfer the shRNA cassette in the retroviral vector. All constructs were verified by DNA sequencing.

### Example 7

### Knockdown of Endogenous TASK 121/BRAF in Melanoma Cells

To address the question of whether BRAF specifically could serve as a therapeutic target for melanoma, we employed the vectors described in Example 6 to deplete the expression of BRAF in cultured melanoma tumor cells. LOX-IMVI cells, derived from a lymph node melanoma metastasis, and A375 cells, from a malignant cutaneous melanoma, were genotyped for BRAF. PCR-amplification and sequencing of exon 15 of BRAF revealed the presence of activated BRAF^{V600E} alleles in both LOX-IMV1 and A375 cell lines. Consistent with this, strong BRAF/MEK/ERK signaling was observed in these cells. This suggested a role for BRAF in their tumorigenic potential.

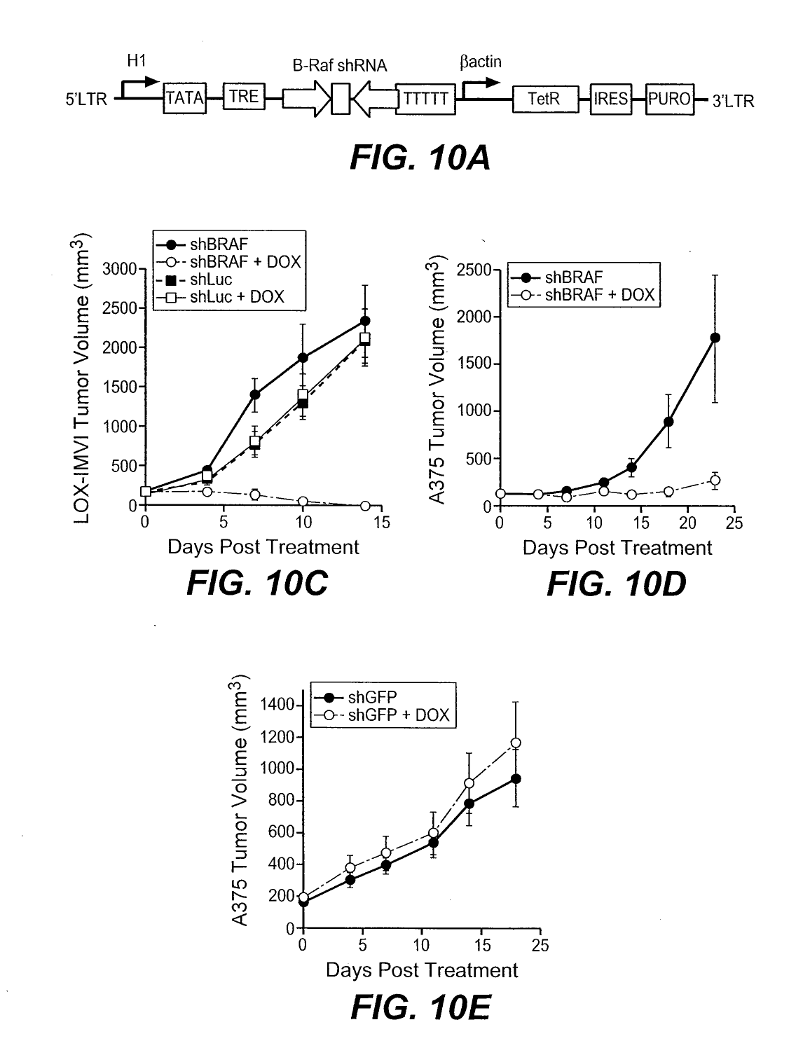

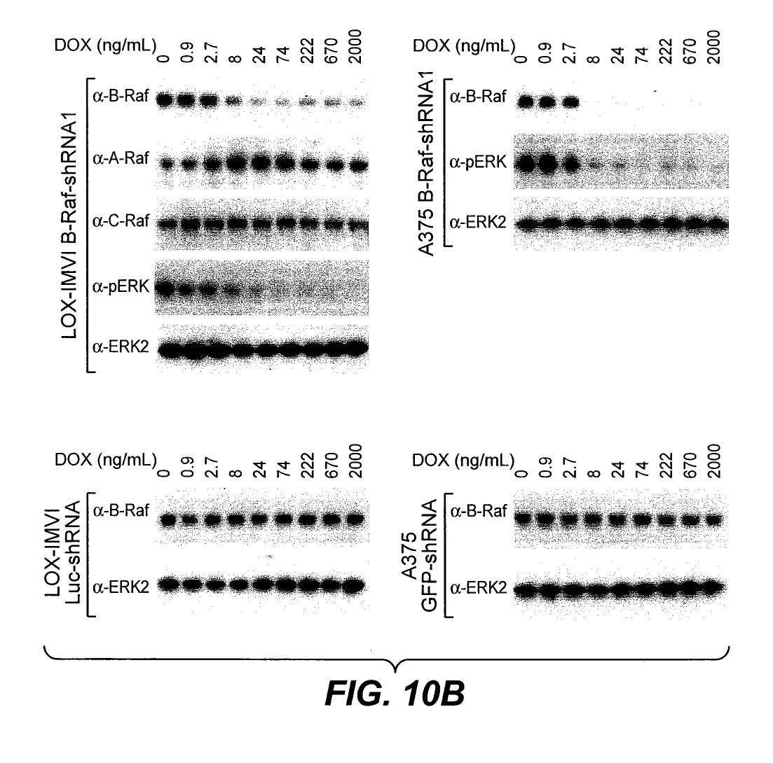

We generated stable cells that expressed the inducible shRNA retroviral vector shown in FIG. 10A (based on pHUSH-GW). Several independent LOX-IMVI and A375 clones were characterized to ensure against a clonal selection bias. In the uninduced state, cells expressing the inducible hairpins were not altered in their baseline growth properties and no discernable background expression could be detected (data not shown). As shown in FIG. 10B, dramatic BRAF suppression was observed in both cell lines by Dox-mediated hairpin targeting of BRAF but not GFP or Luciferase. Densitometry quantitation of immunoblots revealed an effective BRAF protein knockdown of ˜80% and ˜98% for LOX-IMVI and A375, respectively. The suppression of BRAF protein levels was achieved in a dose-dependent manner with a Dox IC₅₀ of approximately 5 ng/ml. BRAF knockdown was reversible and time-dependent, with the maximal mRNA depletion detected 2 days post induction and the corresponding protein depletion occurring at day 3. Induced BRAF-directed shRNA did not diminish the expression of ARAF and RAF1, as has been shown previously for these shRNAs. Consistent with the known BRAF phosphorylation-dependent activation of ERK1/2 via MEK1/2, increasing Dox concentration resulted in a reduction of phospho-ERK1/2 (pERK) while total ERK1/2 levels remain unchanged (FIG. 10B). This indicated that BRAF-mediated ERK1/2 activation and signaling can be abrogated in these cells by Dox-mediated induction of BRAF shRNA.

### Example 8

### Knock-Down of TASK121/BRAF in Tumors in Xenograft Mice Models

Six- to 8-week-old female athymic nu/nu mice or scid-beige mice were purchased from Charles River Laboratories. To assay for BRAF role in subcutaneous tumor models, mice were injected in the right flank with either 3×10⁶ human LOX-IMVI or 1×10⁷ human A375 TASK121-shRNA containing cell clones in 200 μL phosphate-buffered saline (PBS). When tumors reached a mean volume of 100-150 mm³ the mice with similarly sized tumors were grouped into treatment cohorts. Mice received 5% sucrose only or 5% sucrose plus 1-2 mg/ml doxycycline for control and knockdown cohorts, respectively. All water bottles were changed 3 times per week. Tumors were measured with calipers and mice weighed twice per week. Mice whose tumors reach 2000 mm³ were euthanized. At the end of the dosing study, or as indicated, appropriate tumor samples were taken. Each treatment group consisted of 7-10 mice and results are presented as mean tumor volume ±standard deviation (FIGS. 10C-E).

In phenotypic analyses, we first investigated the effects of BRAF knockdown on the in vitro growth of these melanoma cells. Upon Dox addition, LOX-IMVI and A375 cells lacking BRAF show consistent changes in 2D properties as compared with control shRNA-infected cells. These included a delay in cell cycle entry and reduced proliferation and a flattened epithelial-like cell morphology change. Of greater interest, we next tested whether ablation of BRAF function in LOX-IMVI and A375 cells might affect their ability to form tumors in vivo. Inoculation of 3×10⁶ LOX-IMVI cells into immunodeficient nu/nu mice produced large tumors at two weeks (FIG. 10c), consistent with the reported strong tumorigenicity of this cell line. Mice bearing these inducible-shRNA xenografted tumors were administered either 5% sucrose or 2 mg/ml Dox plus 5% sucrose and monitored for tumor progression. Strikingly, knockdown of BRAF completely inhibited LOX-IMVI tumorigenesis in vivo and led to tumor regression (FIG. 10c), even despite the incomplete depletion of BRAF as shown in vitro for the selected clone. Complete responses were observed in 6/10 animals in the Dox treatment cohort. In contrast, sucrose-treated LOX-IMVI/BRAF-shRNA tumors or LOX-IMVI tumors expressing a control shRNA directed against Luciferase continued to increase in size. Similar results were obtained in a second LOX-IMVI study with the same dosing paradigm. There was no difference in body weight between treatment groups and significant Dox-related toxicity was not observed.

We performed a similar in vivo study with A375 melanoma cell lines to further substantiate the role of BRAF in tumorigenesis. In A375 xenografts, BRAF-shRNA induction also halted tumor progression (FIG. 10d), however, the tumors did not regress as observed in the studies involving the LOX-IMVI cell line. There was no discernable effect on tumor growth observed with A375 cells expressing GFP control shRNA (FIG. 10e). Taken together, these results suggest that gain-of-function BRAF signaling was strongly associated with the in vivo tumorigenic ability of melanoma cells and confirmed BRAF as an important therapeutic target.

### Example 9

### TASK121 and the Suppression of Xenograph Tumors

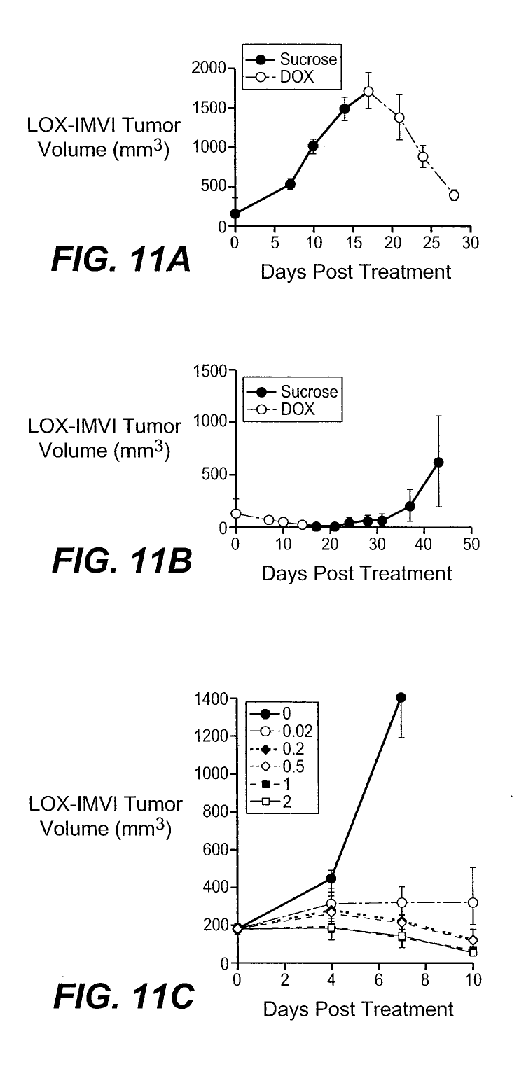

To determine whether inactivation of TASK121 is useful in alleviating melanoma progression, experiments were performed to see if TASK121 reduction induced tumor regression. Moribund mice with ˜1500 mm³ LOX-IMVI/TASK121-shRNA subcutaneous tumor burden (see FIG. 10C, Example 8 above) were given only sucrose in their drinking water and then administered doxycycline at day 15. Within 5 days post TASK121-shRNA induction the tumor volume had visibly decreased and after 2 weeks the tumors had grossly regressed (FIG. 11A), demonstrating that impairment of TASK121 expression can result in rapid elimination of tumor cells. Despite the large starting tumor volume, growth inhibition was equally efficient as compared to early-onset TASK121 knockdown as seen in FIG. 10C of the previous Example.

In a second experiment to determine if LOX-IMVI tumors retained their growth potential, tumors were allowed to establish themselves, TASK121-shRNA was induced for a period of time and then the expression of the shRNA was stopped to see if the tumor would regrow. As shown in FIG. 11B, TASK121 expression in regressing tumors was restored by discontinuing expression of the TASK-shRNA by withdrawing doxycycline. When doxycycline was withdrawn, tumor recurrence was observed in only 2/8 mice (FIG. 11B). In mice where the tumor did not regrow, by the time TASK-121-shRNA induction was stopped by doxycycline removal, the animals no longer had a palpable tumor, indicating that most of the implanted tumor cells in these animals had already been eliminated. This indicated that while TASK121 suppression does not lead to an irreversible tumor regression, the prolonged sustained suppression was sufficient to yield tumor reduction even in well-established tumors.

Mice bearing LOX-IMVI/TASK121-shRNA tumors were treated with 0, 0.02, 0.2, 0.5, 1.0 or 2.0 mg/ml doxycycline and the differences in tumor growth were monitored. Treatment with all tested doxycycline concentrations resulted in stasis or regression, but there was a dose-dependent trend in tumor inhibition in vivo (FIG. 11C). Characterization of the pHUSH vector system suggested that 0.02 mg/ml doxycycline is near the lower threshold for in vivo expression of the shRNA. These results show that even minimal attenuation of TASK121 leads to robust anti-tumor efficacy in these models, suggesting that under physiological conditions there is a broad window for a TASK121 inhibitor to be effective.

### Example 10

### Xenograft Tumor Reduction is TASK121 Specific

The observed result of tumor regression in the xenograft models were the result of TASK121 knockdown, but it remained necessary to confirm that the observed phenotypes result from silencing of the TASK121 target and not from, e.g., unintended, off-target transcripts (Jackson et al., Nat. Biotech. 21:635-637 (2003)). Accordingly, treatment with multiple, different TASK121-specific shRNA was performed to confirm whether the observed changes in melanoma tumor growth were directly linked to TASK121 silencing. The first shRNA used for TASK121 knockdown corresponds to the translated sequence just following the G-loop of the kinase domain (amino acids 461-467 of FIG. 16, SEQ ID NO:2) in which no oncogenic mutations have been described to date. The predominant mutation in TASK121 reported is a valine to glutamic acid change at residue 600 (V600E). This mutation has been found primarily in cutaneous/subcutaneous metastases compared to primary melanomas (Deichmann et al., BMC Cancer 5:58-68 (2005)). Using the rationale that this mutation would be found with high frequency in the clinic, another hairpin specific to a distinct region of the TASK121 transcript (encoding amino acids 597-603 and the V600E mutation) was designed (Hingorani et al., Cancer Res 63:5198-202 (2003)). This second shRNA (TASK121-shRNA2) was cloned into pHUSH, and used to create stable LOX-IMVI clones. These were analyzed by quantitative PCR and Western blotting for knockdown of TASK121 (FIG. 12A). The panel in FIG. 12A shows that TASK121-shRNA2 was as efficient at knocking down TASK121 levels as previous shRNA. The TASK121-shRNA2 was also as equally efficient at attenuating downstream signaling as observed with the TASK121-shRNA1 as previously depicted in FIG. 10B. In xenograft models TASK121-shRNA2-mediated knockdown also showed robust tumor regression upon doxycycline-induction (FIG. 12B). These results supported the conclusion that the silencing of the TASK121 was responsible for the tumor regression, and affirmed the role of TASK121 in tumor growth.

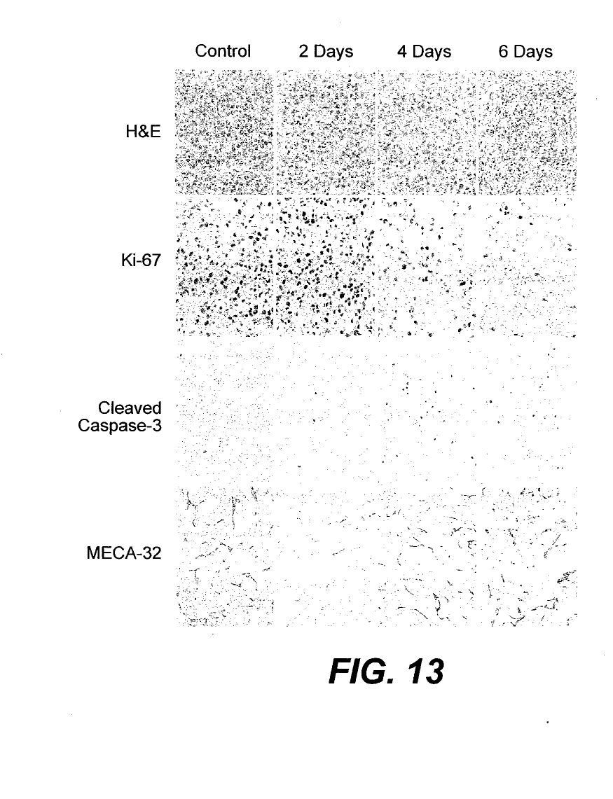

As tumor growth inhibition was the primary endpoint in these studies, histological analysis was performed to define the spectrum of cellular responses that were caused by targeted TASK121 inhibition. Tumors from mice treated with doxycycline for 1 to 7 days (FIG. 12B) were harvested and adjacent sections were probed for expression of Ki-67, activated caspase-3 or MECA-32 (FIG. 13). Formalin-fixed, paraffin-embedded specimens were collected and a routine hematoxylin and eosin slide was first evaluated. IHC staining was performed on 5-μm-thick paraffin-embedded sections using anti-Ki-67, anti-MECA-32 and anti-cleaved caspase-3 antibodies with the streptavidin-biotin system (DakoCytomation, Carpenteria Calif.) according to the manufacturer's instructions. Tissues were counterstained with haematoxylin, dehydrated, and mounted. Compared to xenografts from control animals, tumors from doxycycline-treated mice exhibited fewer neoplastic cells by hematoxylin and eosin visualization. TASK121 does not appear to regulate tumor vascularization as assessed by MECA-32 immunoreactivity of endothelial cells (FIG. 13). Immunohistochemical staining with an antibody to Ki-67 revealed a profound decrease in proliferating cells upon TASK121 knockdown. This result was consistent with the established role of Raf pathway in driving cell cycle progression (Sebolt-Leopold & Herrera, Nat. Rev. Cancer 4:1112-7 (2004)). Tumors also showed increased inflammatory infiltrate and immunophenotyping with B-cell (B220), T-cell (CD3), and macrophage (F480) markers revealed a mild increase in B and T cells and a dramatic increase in macrophages. The presence of inflammation in these xenografts, and particularly macrophages, raised the possibility of a response to apoptotic debris. To confirm this, histochemical staining showed few cleaved caspase-3-positive cells in control tissues, and increased numbers of apoptotic cells following doxycycline treatment after 3 days (and reaching a stable maximum by day 4). Thereafter, the degree of apoptosis was stable from 4 to 7 days post TASK121 shRNA induction. The activation of effector caspases rapidly leads to cell death, so the extent of transient activated capase-3 observed in knockdown tissues indicated a significant degree of apoptosis upon TASK121 knockdown in the context of this tumor model. This result supported the current view that TASK121 mediated signaling plays a role in mediating cell survival. This delay in observable apoptosis was consistent with the 2-3 day requirement for effective TASK121 protein knockdown in vitro and correlates with downstream pathway activation in tumor cells.

### Example 11

### TASK121 Mutations in Cancer

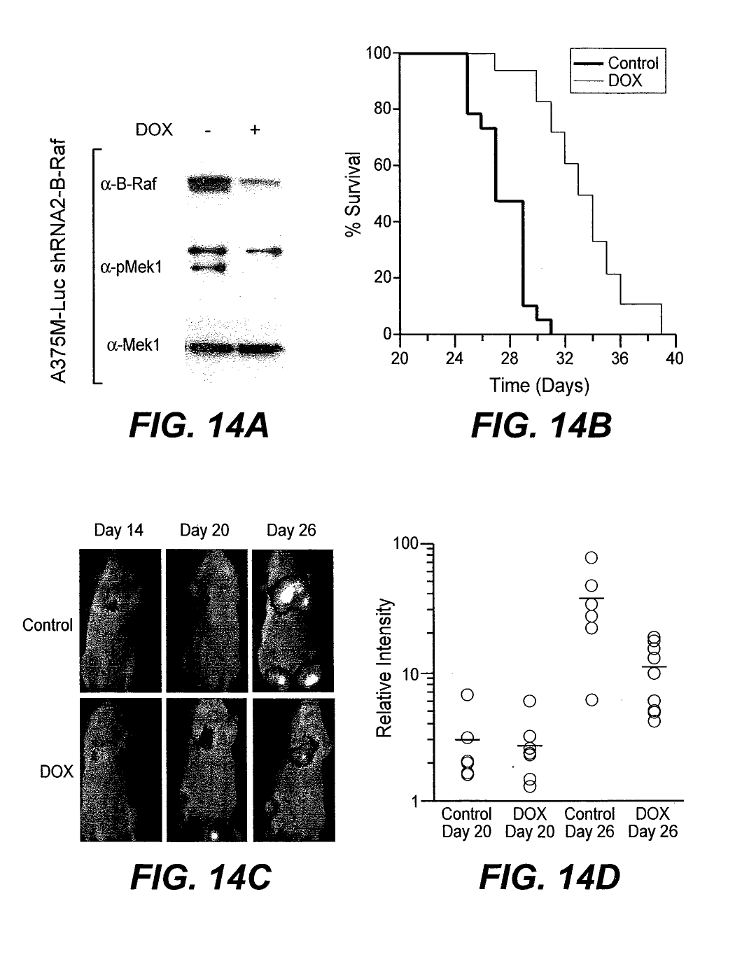

While the dominant mutation found in TASK121 is the V600E change, the extent to which the V600E mutation promotes the growth and metastasis of tumor cells has not yet been experimentally confirmed. Melanoma metastases are very aggressive and are the predominant cause of melanoma-associated death (Rodolfo et al., Cancer Lett. 214:133-147 (2004)), it would be useful to determine if the TASK121 (V600E) mutation played a role in metastatic melanoma. Therefore, A375M cells, which are isolated from an amelanotic melanoma and subsequently selected for high metastatic ability in vivo, were used to evaluate the role of TASK121 in this process (Collisson et al., Mol. Can. Ther. 2:941-948 (2003)). PCR amplification and DNA sequencing of the TASK121 gene in A375 cells determined that these cells contained the V600E mutation. A375 cells were engineered to stably express TASK121-RNAi for doxycycline-regulatable knockdown of TASK121 protein (FIG. 14A) and to concomitantly express firefly luciferase to permit whole-body noninvasive monitoring of tumor development via in vivo bioluminescence imaging (BLI) (Ray et al., Cancer Res 64:1323-1330 (2004)). Pilot in vivo experiments demonstrated that tail vein injection of 4×10⁵ A375M cells into female scid-beige mice led to pulmonary, ovarian and adrenal tumors that are detectable by BLI after a relatively short latency.

To evaluate the efficacy of TASK121 knockdown in this model, cohorts of mice with similarly increasing tumor burden were divided into treatment groups. Scid-beige mice were injected intraperitoneally with 250 RL 200 mg/kg D-luciferin (Biotium, Hayward, Calif.) and anesthetized using isoflurane. During image acquisition in a light-tight box, the animal was maintained on isoflurane via nose cone, and body temperature was maintained using a warming pad. Bioluminescence images were acquired using a cooled intensified charge-coupled device camera in an in vivo imaging system (Xenogen, Alameda, Calif.). Image acquisition times were about 1 minute. Images were processed by co-registering a reference image with a thresholded bioluminescence data image.

Mice were monitored longitudinally for tumor onset, progression, survival and response to TASK121 knockdown. In this mouse melanoma metastasis model, TASK121 knockdown significantly slowed tumor growth and prolonged the survival of mice (p<0.0001, log-rank test). Median survival of all mice treated with 1 mg/ml doxycycline was 33.4 days, in comparison to 27.6 days for control mice and is show in FIG. 14B

For metastatic tumor models, female scid-beige mice were injected intravenously with 50 μL PBS containing 4×10⁵ A375M-luc shRNA cell clones. Tumor progression was monitored by weekly bioluminescence imaging for luciferase and mice were monitored daily for survival. At least 9 mice were used for each cohort. When induction of TASK121 knockdown was delayed until systemic tumors were well established, progression of disease was still partially inhibited as shown by representative images and bioluminescent quantification of pulmonary tumor burden (FIG. 14C, D). These results show that TASK121-shRNA knockdown therapy worked not only to suppress expression in cases where overexpression is causing the tumor, but also in cases where the tumor was caused by an activating mutation. This is a promising strategy to inhibit certain metastatic tumors. These results further indicate that the A375M BLI model can be used to measure the anti-tumor efficacy of anti-TASK121 agents.

### Example 12

### Tumor Screening

Antagonists to TASK polypeptides may be determined in vivo by a nude mouse model. Mammalian cells can be transfected with sufficient amounts of TASK polypeptide expressing plasmid to generate high levels of TASK polypeptide in the cell line. A known number of overexpressing cells can be injected sub-cutaneously into the flank of nude mice. After allowing sufficient time for a tumor to grow and become visible and measurable (typically 2-3 mm in diameter), the mice can be treated with the potential TASK antagonist. To determine if a beneficial effect has occurred, the tumor is measured in millimeters with Vernier calipers, and the tumor burden is calculated; Tumor weight=(length×width²)/2 (Geran, et al., (1972) Cancer Chemotherapy Rep., 31-104). The nude mouse tumor model is a reproducible assay for assessing tumor growth rates and reduction of tumor growth rate by a possible anti-tumor agent in a dose dependant manner. As an example, the compound 317615-HCL, a candidate Protein Kinase Cβ inhibitor, was found to have an anti-tumor effect using this model (Teicher et al., (2002) Can Chemo Pharm 49: 69-77).

### Example 13

### Development of a polII-Driven Inducible microRNA Expression Vector

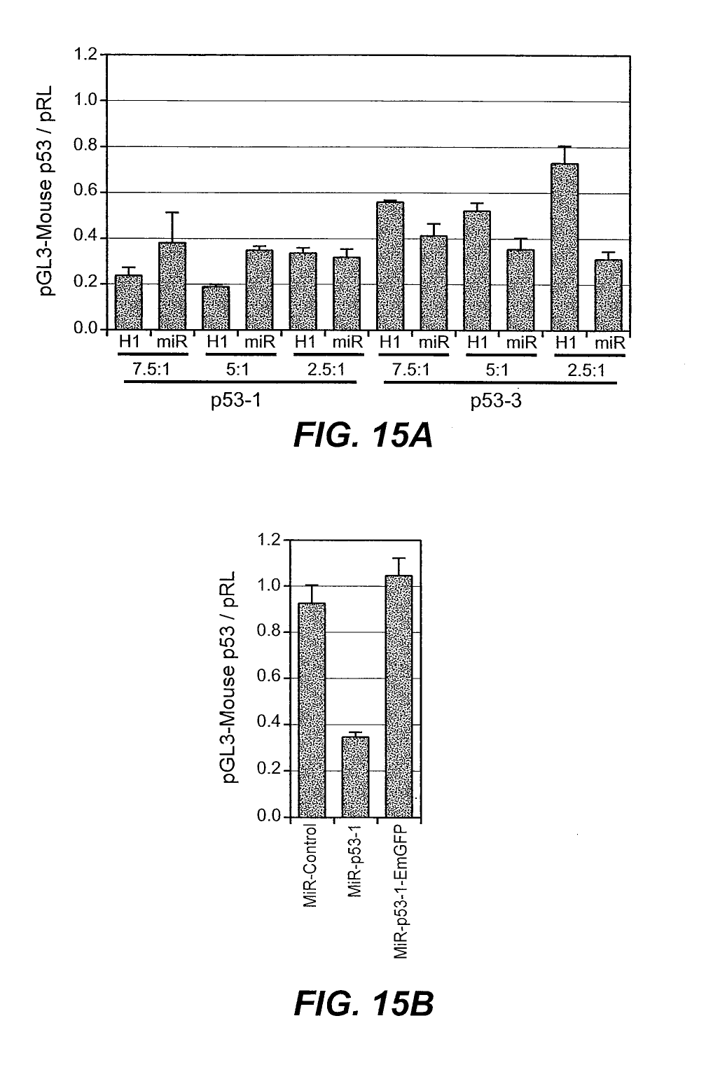

We subcloned a recently developed miRNA expression system, based on the human BIC/miR-155 (Chung, K. H., et al (2006) Nucleic Acids Res, 34, e53), into a vector containing a modified CMV promoter (Yao, F. et al. (1998) Hum Gene Ther, 9, 1939-1950) to yield CMV-TO-EmGFP-miRNA, in which both EmGFP, a green fluorescent protein, and a miRNA are expressed from the same plasmid. The miRNA is processed by Drosha and released from the primary transcript as a pre-miRNA, which then enters the miRNA pathway. The structure of this vector is shown in FIG. 15.

In order to compare the efficacy of shRNA versus miRNA design, two effective miRNAs directed against p53 were converted to H1-shRNAs by identifying the 21-22 nt active siRNA within the miRNA scaffold and then by converting these active siRNAs back to N19 shRNAs. In addition, we examined the effect of having a linker sequence upstream of the miRNA. The following oligonucleotides were used in this study:

Neg

(SEQ ID NO: )

5′-CTGGAGGCTTGCTGAAGGCTGTATGTTATGATTATAACTGTTGTTTT
GGCCACTGACTGACAACAGTTAATCATAACATACAGRACACAAGGCCTGT
TACTAGCACTCACATGGAACAAATGGCC-3′
miR-1-p53

(SEQ ID NO: )

5′-CTGGAGGCTTGCTGAAGGCTGTATGCTGAGTACGTGCACATAACAGA
CTGTTTTGGCCACTGACTGACAGTCTGTTGTGCACGTACTCAGGACACAA
GGCCTGTTACTAGCACTCACATGGAACA-3′
miR-3-p53

(SEQ ID NO: )

5′-5CTGGAGGCTTGCTGAAGGCTGTATGCTGTTAACTCTAAGGCCTCAT
TCAGTTTTGGCCACTGACTGACTGAATGAGCTTAGAGTTAACAGGACACA
AGGCCTGTTACTAGCACTCACATGGAACA-3′
H1-shRNA-Neg

(SEQ ID NO: )

5′-GATCCCCTATGTTATGATTATAACTGTTTTCAAGAGAAACAGTTAAT
CATAACATATTTTTTGGAA-3′
H1-shRNA-p53-1

(SEQ ID NO: )

5′-GATCCCCAGTACGTGCACATAACAGACTTTCAAGAGAAGTCTGTTGT
GCACGTACTTTTTTTGGAAA-3′
H1-shRNA-p53-3

(SEQ ID NO: )

5′-GATCCCCTTAACTCTAAGGCCTCATTCATTCAAGAGATGAATGAGCT
TAGAGTTAATTTTTTGGAAA-3′

We observed significantly increased knockdown by CMV-miR as compared to the corresponding H1-shRNA sequence for the p53-3 sequence (FIG. 15A) at a 2.5 to 1 ratio of CMV-TO-miRNA to pGL3-p53. At molar excess, the performance of the CMV-TO-miR and H1-shRNA hairpins was more similar. For p53-1, we measured a slight increase in knockdown for H1-shRNA at higher transfection ratios, although both vectors performed similarly at lower ratios. Removing the EmGFP leader from CMV-miR-p53-1 destroyed the knockdown efficiency of the original construct (FIG. 15B). Interestingly, when chemically synthesized RNAs corresponding to either the expected H1-shRNA or pre-miR-155 transcripts were transfected, we observed moderated increase in knockdown efficiency with pre-miR-155 RNA for both p53 sequences. To summarize, we have directly compared “first-generation” pol III-shRNA vectors to “second-generation” pot II vectors using the same Dicer-product siRNAs, and we observed an increase in knockdown efficiency for one of the miRNA as compared to the corresponding H1-shRNA version over a range of transfection conditions.

The foregoing written specification is considered to be sufficient to enable one skilled in the art to practice the invention. Indeed, various modifications of the invention in addition to those shown and described herein will become apparent to those skilled in the art from the foregoing description and fall within the scope of the appended claims. All references cited herein, including patent applications and publications, are incorporated by reference in their entirety.

What is claimed is:

1. A polynucleotide comprising (a) a polynucleotide encoding a codon optimized Tet repressor (TetR) protein and (b) a promoter comprising a tet operon sequence.

2. The polynucleotide of claim 1, wherein the codon optimized TetR is optimized for mammalian expression.

3. The polynucleotide of claim 1, wherein the polynucleotide encoding codon optimized TetR is:
(a) a polynucleotide shown in FIG. 17 (SEQ ID NO:1);
(b) a polynucleotide comprising a polynucleotide that hybridizes under stringent conditions to the compliment of the polynucleotide shown in FIG. 17 (SEQ ID NO:1); or
(c) a polynucleotide comprising a polynucleotide that is 90% identical to the polynucleotide shown in FIG. 17 (SEQ ID NO:1).

4. The polynucleotide of claim 1, wherein the promoter is a RNA polymerase III promoter.

5. The polynucleotide of claim 4, wherein the promoter is a H1 promoter.

6. The polynucleotide of claim 1, wherein the promoter comprises:
(a) a polynucleotide shown in SEQ ID NO:19;
(b) a polynucleotide comprising a polynucleotide that hybridizes under stringent conditions to the compliment of the polynucleotide shown in SEQ ID NO:19; or
(c) a polynucleotide comprising a polynucleotide that is 90% identical to the polynucleotide shown in
SEQ ID NO:19.

7. The polynucleotide of claim 3, wherein the promoter comprises:
(a) a polynucleotide shown in SEQ ID NO:19;
(b) a polynucleotide comprising a polynucleotide that hybridizes under stringent conditions to the compliment of the polynucleotide shown in SEQ ID NO:19; or
(c) a polynucleotide comprising a polynucleotide that is 90% identical to the polynucleotide shown in SEQ ID NO:19.

8. The polynucleotide of claim 7, wherein the promoter is operably linked to a RNA coding sequence.

9. The polynucleotide of claim 8, wherein the RNA coding sequence encodes a self-complimentary RNA molecule having a sense region, an antisense region and a loop region.

10. The polynucleotide of claim 9, wherein the sense region and antisense region are each between about 15 and 30 nucleotides in length.

11. The polynucleotide of claim 10, wherein the loop region is about 2 to about 15 nucleotides in length.

12. The polynucleotide of claim 8, wherein the polynucleotide further comprises at least one termination sequence operably linked to the RNA coding region.

13. The polynucleotide of claim 7, wherein the polynucleotide comprises a first RNA coding region operably linked to a first RNA Polymerase III promoter and a second RNA coding region operably linked to a second RNA Polymerase III promoter

14. The polynucleotide of claim 7, wherein the polynucleotide comprises a first RNA Polymerase III promoter and a second RNA Polymerase III promoter, each operably linked to the RNA coding region, such that expression of the RNA coding region from the first RNA Polymerase III promoter results in the synthesis of a first RNA molecule and expression of the RNA coding region from the second RNA Polymerase III promoter results in the synthesis of a second RNA molecule substantially complementary to the first RNA molecule.

15. The polynucleotide of claim 8, wherein expression of the RNA coding region results in the down regulation of a target gene, wherein the target gene comprises a sequence that is at least about 90% identical with the RNA coding region.

16. The polynucleotide of claim 1, wherein the promoter is a RNA polymerase II promoter.

17. The polynucleotide of claim 16, wherein the promoter is the CMV promoter.

18. The polynucleotide of claim 16, wherein the promoter is operably linked to a polynucleotide encoding a selected sequence.

19. The polynucleotide of claim 17, wherein the selected sequence is a selectable marker.

20. The polynucleotide of claim 1, wherein the codon optimized TetR is operably linked to a second promoter.

21. The polynucleotide of claim 20, wherein the second promoter is a RNA polymerase II promoter.

22. The polynucleotide of claim 21, wherein the second promoter is a human beta-actin promoter or a CMV promoter.

23. The polynucleotide of claim 20, wherein the second promoter is further operably linked to a polynucleotide encoding a selected sequence.

24. The polynucleotide of claim 23, wherein the selected sequence encodes a selectable marker.

25. The polynucleotide of claim 8, wherein the promoter is a RNA polymerase II promoter.

26. The polynucleotide of claim 25, wherein the RNA polymerase II promoter is the CMV promoter.

27. The polynucleotide of claim 25, wherein the RNA polymerase II promoter is operably linked to a polynucleotide encoding a selected sequence.

28. The polynucleotide of claim 27, wherein the selected sequence is a selectable marker.

29. The polynucleotide of claim 8, wherein the codon optimized TetR is operably linked to a second promoter.

30. The polynucleotide of claim 29, wherein the second promoter is a RNA polymerase II promoter.

31. The polynucleotide of claim 30, wherein the second promoter is a human beta-actin promoter.

32. The polynucleotide of claim 30, wherein the second promoter is further operably linked to a polynucleotide encoding a selected sequence.

33. The polynucleotide of claim 32, wherein the selected sequence encodes a selectable marker.

34. The polynucleotide of any of claims 1, 3, 6, 7, or 8, wherein the polynucleotide further comprises R and U5 sequence from a 5′ lentiviral long terminal repeat, and a self-inactivating lentiviral 3′LTR.

35. The polynucleotide of claim 34, wherein the 5′ LTR sequences are from human immunodeficiency virus (HIV).

36. The polynucleotide of claim 34, wherein the polynucleotide comprises a woodchuck hepatitis virus enhancer element sequence.

37. The polynucleotide of claim 34, wherein the polynucleotide comprises a cPPT element sequence.

38. The polynucleotide of claim 34, wherein the self-inactivating 3′ LTR comprises a U3 element with a deletion of its enhancer sequence.

39. The polynucleotide of claim 34, wherein the self-inactivating 3′ LTR is a modified HIV 3′ LTR.

40. A polynucleotide comprising:
(a) a first transcription unit comprising a RNA polymerase III promoter, wherein the RNA polymerase III promoter comprises a polynucleotide sequence shown in SEQ ID NO:19, and wherein the RNA polymerase III promoter is operably linked to a RNA coding sequence; and
(b) a second transcription unit comprising a second promoter, wherein the second promoter is operably linked to (1) a polynucleotide encoding a TetR protein, and (2) a selectable marker.

41. The polynucleotide of claim 40, wherein the polynucleotide encoding the TetR protein is the polynucleotide shown in FIG. 17 (SEQ ID NO:1).

42. The polynucleotide of claim 40, wherein the RNA coding sequence encodes a self-complimentary RNA molecule having a sense region, an antisense region and a loop region.

43. The polynucleotide of claim 42, wherein the sense region and antisense region are each between about 15 and 30 nucleotides in length.

44. The polynucleotide of claim 43, wherein the loop region is about 2 to about 15 nucleotides in length.

45. A polynucleotide comprising:
(a) a first transcription unit comprising a RNA polymerase II promoter, wherein the RNA polymerase II promoter comprises a polynucleotide sequence shown in SEQ ID NO:19, and wherein the RNA polymerase II promoter is operably linked to a RNA coding sequence; and
(b) a second transcription unit comprising a second promoter, wherein the second promoter is operably linked to (1) a polynucleotide encoding a TetR protein, and (2) a selectable marker.

46. The polynucleotide of claim 45, wherein the polynucleotide encoding the TetR protein is the polynucleotide shown in FIG. 17 (SEQ ID NO:1).

47. The polynucleotide of claim 46, wherein the RNA coding sequence encodes a miRNA.

48. A polynucleotide comprising:
(a) a first transcription unit comprising a RNA polymerase II promoter, wherein the RNA polymerase II promoter comprises a polynucleotide sequence shown in SEQ ID NO:19, and wherein the RNA polymerase II promoter is operably linked to a polynucleotide encoding a selected sequence; and
(b) a second transcription unit comprising a second promoter, wherein the second promoter is operably linked to (1) a polynucleotide encoding a TetR protein, and (2) a selectable marker.

49. The polynucleotide of claim 48, wherein the polynucleotide encoding the TetR protein is the polynucleotide shown in FIG. 17 (SEQ ID NO:1).

50. The polynucleotide of any of claims 40, 45, or 48, wherein the polynucleotide further comprises a retroviral 5′ LTR and 3′LTR.

51. The polynucleotide of any of claims 40, 45, or 48, wherein the polynucleotide further comprises a R and U5 sequence from a 5′ lentiviral long terminal repeat, and a self-inactivating lentiviral 3′LTR.

52. The polynucleotide of any of claims 40, 45, or 48, wherein the second transcription unit comprises, in order from 5′ to 3′: a promoter, a polynucleotide encoding a TetR, an IRES, and a polynucleotide encoding a selectable marker.

53. The polynucleotide of any of claims 40, 45, or 48, wherein the second transcription unit comprises, in order from 5′ to 3′: a promoter, a polynucleotide encoding a selectable marker, an IRES, and a polynucleotide encoding a TetR.

54. A polynucleotide comprising:
(a) a polynucleotide shown in FIG. 17 (SEQ ID NO:1);
(b) a polynucleotide comprising a polynucleotide that hybridizes under stringent conditions to a compliment of the polynucleotide shown in FIG. 17 (SEQ ID NO:1);
(c) a polynucleotide comprising a polynucleotide that is 90% identical to the polynucleotide shown in FIG. 17 (SEQ ID NO:1); or
(d) a complement of a polynucleotides shown in (a), (b) or (c).

55. A polynucleotide comprising:
(a) a polynucleotide shown in SEQ ID NO:19;
(b) a polynucleotide comprising a polynucleotide that hybridizes under stringent conditions to a compliment of the polynucleotide shown in SEQ ID NO:19;
(c) a polynucleotide comprising a polynucleotide that is 90% identical to the polynucleotide shown in SEQ ID NO:19; or
(d) a complement of the polynucleotide shown in (a), (b) or (c).

56. A vector comprising the polynucleotide of any of claims 1, 3, 6, 7, 8, 40, 45, or 48.

57. A cell comprising the polynucleotide of any of claims 1, 3, 6, 7, 8, 40, 45, or 48.

58. A method for expressing a selected sequence within a cell, said method comprising: introducing the polynucleotide of any of claims 1, 3, 6, 7, 8, 40, 45, 48 or 50-53 into the cell; and (b) treating the cell with an induction agent, whereby the selected sequence is expressed within the cell.

59. The method of claim 57 or 58, wherein the induction agent is tetracycline or doxyxycline.


SEQUENCE LISTING (26 sequences)

SEQ ID NO: 1 - DNA, 656 bp, Artificial sequence
  Codon-optomized TetR
atgtccagactggataagtccaaggtgattaattccgctctggaactcctgaacgaggtcggcatcgagggactgaccacacggaagctggctcagaaactcggcgtcgaacagcctaccctctactggcatgtcaaaaataagagagccctcctggacgccctggctatcgagatgctggacagacaccacacccacttctgccccctggaaggcgaatcctggcaggatttcctccggaacaacgctaaaagctttagatgcgccctcctcagccatagagacggagctaaagtgcacctgggaacccggcctacagaaaaacagtacgagacactggaaaaccagctcgctttcctctgccaacaaggctttagcctggaaaacgccctctacgctctcagcgctgtcggccattttacactgggctgcgtgctcgaggaccaggagcaccaagtggctaaagagagcgggaaacccctaccaccgatagcatgccccccctgctgagacaagccattgagctctttgatcatcagggagctgaacccgccttcctctttggactcgaactcattatttgcggactcgagaagcaactgaaatgcgaaagcggaagcgcctactccggctccagagaatttcggtcctactag

SEQ ID NO: 2 - protein, 169 aa, Artificial sequence
  Codon-optimized TetR
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKESGKPLPPIACPPC

SEQ ID NO: 3 - DNA, 2721 bp, Artificial sequence
  eGFP-Melk fusion
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccaaagattatgatgaacttctcaaatattatgaattacatgaaactattgggacaggtggctttgcaaaggtcaaacttgcctgccatatccttactggagagatggtagctataaaaatcatggataaaaacacactagggagtgatttgccccggatcaaaacggagattgaggccttgaagaacctgagacatcagcatatatgtcaactctaccatgtgctagagacagccaacaaaatattcatggttcttgagtactgccctggaggagagctgtttgactatataatttcccaggatcgcctgtcagaagaggagacccgggttgtcttccgtcagatagtatctgctgttgcttatgtgcacagccagggctatgctcacagggacctcaagccagaaaatttgctgtttgatgaatatcataaattaaagctgattgactttggtctctgtgcaaaacccaagggtaacaaggattaccatctacagacatgctgtgggagtctggcttatgcagcacctgagttaatacaaggcaaatcatatcttggatcagaggcagatgtttggagcatgggcatactgttatatgttcttatgtgtggatttctaccatttgatgatgataatgtaatggctttatacaagaagattatgagaggaaaatatgatgttcccaagtggctctctcccagtagcattctgcttcttcaacaaatgctgcaggtggacccaaagaaacggatttctatgaaaaatctattgaaccatccctggatcatgcaagattacaactatcctgttgagtggcaaagcaagaatccttttattcacctcgatgatgattgcgtaacagaactttctgtacatcacagaaacaacaggcaaacaatggaggatttaatttcactgtggcagtatgatcacctcacggctacctatcttctgcttctagccaagaaggctcggggaaaaccagttcgtttaaggctttcttctttctcctgtggacaagccagtgctaccccattcacagacatcaagtcaaataattggagtctggaagatgtgaccgcaagtgataaaaattatgtggcgggattaatagactatgattggtgtgaagatgatttatcaacaggtgctgctactccccgaacatcacagtttaccaagtactggacagaatcaaatggggtggaatctaaatcattaactccagccttatgcagaacacctgcaaataaattaaagaacaaagaaaatgtatatactcctaagtctgctgtaaagaatgaagagtactttatgtttcctgagccaaagactccagttaataagaaccagcataagagagaaatactcactacgccaaatcgttacactacaccctcaaaagctagaaaccagtgcctgaaagaaactccaattaaaataccagtaaattcaacaggaacagacaagttaatgacaggtgtcattagccctgagaggcggtgccgctcagtggaattggatctcaaccaagcacatatggaggagactccaaaaagaaagggagccaaagtgtttgggagccttgaaagggggttggataaggttatcactgtgctcaccaggagcaaaaggaagggttctgccagagacgggcccagaagactaaagcttcactataatgtgactacaactagattagtgaatccagatcaactgttgaatgaaataatgtctattcttccaaagaagcatgttgactttgtacaaaagggttatacactgaagtgtcaaacacagtcagattttgggaaagtgacaatgcaatttgaattagaagtgtgccagcttcaaaaacccgatgtggtgggtatcaggaggcagcggcttaagggcgatgcctgggtttacaaaagattagtggaagacatcctatctagctgcaaggtataa

SEQ ID NO: 4 - protein, 906 aa, Artificial sequence [sequence omitted]

SEQ ID NO: 5 - DNA, 1653 bp, Artificial sequence
  Melk kinase-luciferase fusion
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 6 - protein, 550 aa, Artificial sequence
  Melk kinase-luciferase fusion
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 7 - DNA, 68 bp, Artificial sequence
  oligonucleotide
acgtgaaatccctatcagtgatagagacttataagttccctatcagtgatagagatctaaagggaaaa

SEQ ID NO: 8 - DNA, 72 bp, Artificial sequence
  oligonucleotide
agctttttccctttagatctctatcactgatagggaacttataagtctctatcactgatagggatttcacgt

SEQ ID NO: 9 - DNA, 65 bp, Artificial sequence
  shLUC-GL3
gatcccccttacgctgagtacttcgattcaagagatcgaagtactcagcgtaagttttttggaaa

SEQ ID NO: 10 - DNA, 64 bp, Artificial sequence
  shMELK-B
gatcccgagattagtggaagatatcttcaagagagatatcttccactaatctcttttttggaaa

SEQ ID NO: 11 - DNA, 65 bp, Artificial sequence
  TASK121 shRNA-1 (sense)
gatccccagaattggatctggatcatttcaagagaatgatccagatccaattctttttttggaaa

SEQ ID NO: 12 - DNA, 65 bp, Artificial sequence
  TASK121 shRNA-1 (antisense)
agcttttccaaaaaaagaattggatctggatcattctcttgaaatgatccagatccaattctggg

SEQ ID NO: 13 - DNA, 65 bp, Artificial sequence
  TASK121 shRNA-2 (sense)
gatccccgctacagagaaatctcgatttcaagagaatcgagatttctctgtagcttttttggaaa

SEQ ID NO: 14 - DNA, 65 bp, Artificial sequence
  TASK121 shRNA-2 (antisense)
agcttttccaaaaaagctacagagaaatctcgattctcttgaaatcgagatttctctgtagcggg

SEQ ID NO: 15 - DNA, 65 bp, Artificial sequence; identical to SEQ ID NO: 9
  Luciferase shRNA (sense)

SEQ ID NO: 16 - DNA, 65 bp, Artificial sequence
  Luciferase shRNA (antisense)
agcttttccaaaaaacttacgctgagtacttcgatctcttgaatcgaagtactcagcgtaagggg

SEQ ID NO: 17 - DNA, 67 bp, Artificial sequence
  sequence is synthesized
gatccccagatccgccacaacatcgattcaagagatcgatgttgtggcggatcttgttttttggaaa

SEQ ID NO: 18 - DNA, 67 bp, Artificial sequence
  EGFP shRNA (antisense)
agcttttccaaaaaacaagatccgccacaacatcgatctcttgaatcgatgttgtggcggatctggg

SEQ ID NO: 19 - DNA, 217 bp, Artificial sequence
  2X-tet02-H1 promoter segment
cgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatag

SEQ ID NO: 20 - DNA, 6 bp, Artificial sequence
  Termination sequence
tttttt

SEQ ID NO: 21 - DNA, 125 bp, Artificial sequence
  Neg
ctggaggcttgctgaaggctgtatgttatgattataactgttgttttggccactgactgacaacagttaatcataacatacagracacaaggcctgttactagcactcacatggaacaaatggcc

SEQ ID NO: 22 - DNA, 125 bp, Artificial sequence
  miR-1-p53
ctggaggcttgctgaaggctgtatgctgagtacgtgcacataacagactgttttggccactgactgacagtctgttgtgcacgtactcaggacacaaggcctgttactagcactcacatggaaca

SEQ ID NO: 23 - DNA, 125 bp, Artificial sequence
  miR-3-p53
ctggaggcttgctgaaggctgtatgctgttaactctaaggcctcattcagttttggccactgactgactgaatgagcttagagttaacaggacacaaggcctgttactagcactcacatggaaca

SEQ ID NO: 24 - DNA, 66 bp, Artificial sequence
  H1-shRNA-Neg
gatcccctatgttatgattataactgttttcaagagaaacagttaatcataacatattttttggaa

SEQ ID NO: 25 - DNA, 67 bp, Artificial sequence
  H1-shRNA-p53-1
gatccccagtacgtgcacataacagactttcaagagaagtctgttgtgcacgtactttttttggaaa

SEQ ID NO: 26 - DNA, 67 bp, Artificial sequence
  H1-shRNA-p53-3
gatccccttaactctaaggcctcattcattcaagagatgaatgagcttagagttaattttttggaaa